(19) United States
(12) Patent Application Publication
(10) Pub. No.: US 2010/0047309 A1
(43) Pub. Date: Feb. 25, 2010

(54) Graft collar and scaffold apparatuses for musculoskeletal tissue engineering and related methods

(76) Inventors: Helen H. Lu, New York, NY (US); Jeffrey P. Spalazzi, Staten Island, NY (US); Moira C. Vyner, Greenwich, CT (US); Kristen L. Moffat, Oakmont, PA (US)

Correspondence Address: COOPER & DUNHAM, LLP, 30 Rockefeller Plaza, 20th Floor, NEW YORK, NY, 10112, US

(21) Appl. No.: 12/455,765

(22) Filed: Jun. 6, 2009

Related U.S. Application Data
(63) Continuation-in-part of application No. PCT/US2008/010985, filed on Sep. 22, 2008.
Application No. PCT/US2008/010985 is a continuation-in-part of application No. PCT/US2008/007323, filed on Jun. 11, 2008.
Application No. PCT/US2008/007323 is a continuation-in-part of application No. PCT/US2007/025127, filed on Dec. 6, 2007.
(60) Provisional application No. 60/873,518, filed on Dec. 6, 2006.
(60) Provisional application No. 60/934,198, filed on Jun. 11, 2007.
(60) Provisional application No. 60/994,745, filed on Sep. 21, 2007.

(51) Int. Cl. A61F 2/08; A61K 38/18; A61K 35/00; A61K 35/32; C08J 5/00

(52) U.S. Cl. 424/423; 623/13.14; 514/12; 424/93.7; 264/331.11

(57) ABSTRACT

This application describes apparatuses and methods for musculoskeletal tissue engineering. Specifically, graft collar and scaffold apparatuses are provided for promoting fixation of musculoskeletal soft tissue to bone.

This application provides for graft collars comprising biopolymer mesh and/or polymer-fiber mesh for fixing tendon to bone. In one aspect, the graft collar comprises more than one region, wherein the regions can comprise different materials configured to promote integration of and the regeneration of the interfacial region between tendon and bone.

This application also provides for scaffold apparatuses and methods for fixing musculoskeletal soft tissue to bone. The scaffold apparatus is multiphasic, preferably triphasic, and each phase is configured promote growth and proliferation of a different cell and its associated tissue. In one aspect, the scaffold apparatus is triphasic, with phases comprising materials to promote growth and proliferation of fibroblasts, chondroblasts, and osteoblasts. In addition, an apparatus comprising two portions, each of said portion being the scaffold apparatus described above is provided, wherein each of said portion encases one end of a soft tissue graft. Further, a triphasic interference screw is provided.

This application further provides apparatuses and methods for inducing formation of fibrocartilage comprising wrapping a graft collar with polymer-fiber mesh configured to apply compression to the graft collar. In another aspect, the polymer-fiber is applied directly to the graft to apply compression to the graft.

This application is a continuation-in-part of PCT International Application No. PCT/US2008/010985, filed Sep. 22, 2008, PCT International Application No. PCT/US2008/007323, filed Jun. 11, 2008 and PCT International Application No. PCT/US2007/025127, filed Dec. 6, 2007, the entire contents of each of which are hereby incorporated by reference herein.

Throughout this application, certain publications are referenced. Full citations for these publications, as well as additional related references, may be found immediately preceding the claims. The disclosures of these publications are hereby incorporated by reference into this application in order to more fully describe the state of the art as of the date of the invention described and claimed herein.

### BACKGROUND OF THE INVENTION

This application relates to musculoskeletal tissue engineering. Some exemplary embodiments which include a soft tissue-bone interface are discussed.

As an example of a soft tissue-bone interface, the human anterior cruciate ligament (ACL) is described below. The ACL and ACL-bone interface are used in the following discussion as an example and to aid in understanding the description of the methods and apparatuses of this application. This discussion, however, is not intended to, and should not be construed to, limit the claims of this application.

The ACL consists of a band of regularly oriented, dense connective tissue that spans the junction between the femur and tibia. It participates in knee motion control and acts as a joint stabilizer, serving as the primary restraint to anterior tibial translation. The natural ACL-bone interface consists of three regions: ligament, fibrocartilage (non-mineralized and mineralized) and bone. The natural ligament to bone interface is arranged linearly from ligament to fibrocartilage and to bone. The transition results in varying cellular, chemical, and mechanical properties across the interface, and acts to minimize stress concentrations from soft tissue to bone.

The ACL is the most often injured ligament of the knee. (Johnson, 1982) with over 300,000 ACL injuries reported (Gotlin, 2000) and more than 100,000 reconstruction procedures performed annually (American Academy of Orthopaedic Surgeons, 1997) in the United States. Due to its inherently poor healing potential and limited vascularization, ACL ruptures do not heal effectively upon injury, and surgical intervention is typically needed to restore normal function to the knee.

### ACL Grafts

Clinically, autogenous grafts based on either bone-patellar tendon-bone (BPTB) grafts or hamstring-tendon (HST) grafts are often a preferred grafting system for ACL reconstruction, primarily due to a lack of alternative grafting solutions.

Primary ACL reconstruction has traditionally been based on BPTB grafts, with a shift in recent years toward the utilization of semitendinosus or HST grafts (Goldblatt, 2005; Sherman, 2004; Wagner, 2005) due to the high incidence of donor site morbidity and complications related to the harvest of BPTB grafts. Allografts are also routinely utilized for ACL reconstruction (Grossman, 2005; Johnson, 2003), especially with advancements in allograft processing and comprehensive studies demonstrating comparable clinical outcomes between allogeneic and autologous grafts (Indelli, 2004; McGuire, 2003; Peterson, 2001; Poehling, 2005; Shelton, 1997). Examples of allogeneic grafts used include the patellar, Achilles, anterior or posterior tibialis, semitendinosus or gracilis, and quadriceps tendons, with the tibialis and Achilles tendons being the most common (Grossman, 2005; Indelli, 2004; Peterson, 2001; Poehling, 2005; Shelton, 1997; Vanderploeg, 2004). Historically, BPTB graft has been the gold standard for ACL reconstruction in part due to its ability to integrate with subchondral bone via the bony ends. Moreover, it possesses intact insertion sites which can serve as functional transitions between soft tissue and bone. In contrast, the autologous hamstring tendon graft and tendon allografts are fixed mechanically within the femoral bone tunnel by passing the tendon around a transfemoral pin, while an interference screw with a washer or staple is used to fix the graft within the tibial bone tunnel. Currently, the primary cause of failure for these tendon-based grafts is their inability to integrate with subchondral bone through an anatomic soft tissue-to-bone interface (Anderson, 2001; Blickenstaff, 1997; Chen, 2003; Fu, 2000; Grana, 1994; Johnson, 1982; Liu, 1997; Panni, 1997; Rodeo, 1993; Thomopoulos, 2002; Weiler, 2002; Yoshiya, 2000). It has been reported that the lack of graft integration within the bone tunnels contributes to the sub-optimal clinical outcome of semitendinosus grafts (Friedman, 1985; Jackson, 1987; Yahia, 1997). Post-operative tendon-to-bone healing does not result in the complete re-establishment of the normal transition zones of the native ACL-to-bone enthesis (Anderson, 2001; Batra, 2002; Blickenstaff, 1997; Chen, 2003; Chen, 1997; Eriksson, 2000; Grana, 1994; Liu, 1997; Malinin, 2002; Panni, 1997; Rodeo, 1993; Song, 2004; Thomopoulos, 2002; Yoshiya, 2000). Rather, a non-anatomic fibrovascular scar tissue forms at the graft and bone junction within the bone tunnel (Rodeo, 2001; Rodeo, 1993; Rodeo, 1999). Consequently, the tendon graft-to-bone interface represents the weak link of the reconstructed ACL graft (Kurosaka, 1987).

Current ACL grafts are also limited by donor site morbidity, tendonitis and arthritis. Synthetic grafts may exhibit good short term results but encounter clinical failure in long-term follow-ups, since they are unable to duplicate the mechanical strength and structural properties of human ACL tissue. ACL tears and ruptures are therefore commonly repaired using semitendinosus grafts. Although semitendinosus autografts are superior, they often fail at the insertion site between the graft and the bone tunnel. One of the major causes of failure in this type of reconstruction grafts is its inability to regenerate the soft-tissue to bone interface.

Despite their distinct advantages over synthetic substitutes, autogenous grafts have a relatively high failure rate. A primary cause for the high failure rate is the lack of consistent graft integration with the subchondral bone within bone tunnels. The site of graft contact in femoral or tibial tunnels represents the weakest point mechanically in the early post-operative healing period. Therefore, success of ACL reconstructive surgery depends heavily on the extent of graft integration with bone.

ACL reconstruction based on autografts often results in loss of functional strength from an initial implantation time, followed by a gradual increase in strength that does not typically reach the original magnitude. Despite its clinical success, long term performance of autogenous ligament substitutes are dependent on several factors, including the structural and material properties of the graft, the initial graft tension (Beynnon, 1996; Beynnon, 1997; Fleming, 1992; Flemming, 2001; Gregor, 1994; Shapiro, 1997), the intra-articular position of the graft (Loh, 2003; Markolf, 2002), and graft fixation (Kurosaka, 1987; Robertson, 1986). These grafts typically do not achieve normal restoration of ACL morphology and knee stability.

There is often a lack of graft integration with host tissue, in particular at bony tunnels, which contributes to suboptimal clinical outcome of these grafts. The fixation sites at the tibial and femoral tunnels, instead of the isolated strength of the graft material, have been identified as mechanically weak points in the reconstructed ACL. Poor graft integration may lead to enlargement of the bone tunnels, and in turn may compromise the long term stability of the graft.

Increased emphasis has been placed on graft fixation, as post surgery rehabilitation protocols require the immediate ability to exercise full range of motion, reestablish neuromuscular function and weight bearing. (Brand, 2000; Rodeo, 1993) During ACL reconstruction, the bone-patellar tendon-bone or hamstring-tendon graft is fixed into the tibial and femoral tunnels using a variety of fixation techniques. Fixation devices include, for example, staples, screw and washer, press fit EndoButton® devices, and interference screws. In many instances, EndoButton® devices or Mitek® Anchor devices are utilized for fixation of femoral insertions. Staples, interference screws, or interference screws combined with washers can be used to fix the graft to the tibial region.

The interference screw is a standard device for graft fixation. The interference screw, about 9 mm in diameter and at least 20 mm in length, is used routinely to secure tendon to bone and bone to bone in ligament reconstruction. Surgically, the knee is flexed and the screw is inserted from the para-patellar incision into the tibial socket, and the tibial screw is inserted just underneath the joint surface. After tension is applied to the femoral graft and the knee is fully flexed, the femoral tunnel screw is inserted. This procedure has been reported to result in stiffness and fixation strength levels which are adequate for daily activities and progressive rehabilitation programs.

While the use of interference screws have improved the fixation of ACL grafts, mechanical considerations and biomaterial-related issues associated with existing screw systems have limited the long term functionality of the ligament substitutes. Screw-related laceration of either the ligament substitute or bone plug suture has been reported. In some cases, tibial screw removal was necessary to reduce the pain suffered by the patient. Stress relaxation, distortion of magnetic resonance imaging, and corrosion of metallic screws have provided motivation for development of biodegradable screws based on poly-α-hydroxy acids. While lower incidence of graft laceration was reported for biodegradable screws, the highest interference fixation strength of the grafts to bone is reported to be 475 N, which is significantly lower than the attachment strength of ACL to bone. When tendon-to-bone fixation with polylactic acid-based interference screws was examined in a sheep model, intraligamentous failure was reported by 6 weeks. In addition, fixation strength is dependent on quality of bone (mineral density) and bone compression.

### Zones in the Fibrocartilage Interface

Two insertion zones can be found in the ACL, one at the femoral end and another located at the tibial attachment site. The ACL can attach to mineralized tissue through insertion of collagen fibrils, and there exists a gradual transition from soft tissue to bone. The femoral attachment area in the human ACL was measured to be 113±27 mm² and 136±33 mm² for the tibia insertion. With the exception of the mode of collagen insertion into the subchondral bone, the transition from ACL to bone is histologically similar for the femoral and tibial insertion sites.

The insertion site is comprised of four different zones: ligament, non-mineralized fibrocartilage, mineralized fibrocartilage, and bone. The first zone, which is the ligament proper, is composed of solitary, spindle-shaped fibroblasts aligned in rows, and embedded in parallel collagen fibril bundles of 70-150 μm in diameter. Primarily type I collagen makes up the extracellular matrix, and type III collagen, which are small reticular fibers, are located between the collagen I fibril bundles. The second zone, which is fibro-cartilaginous in nature, is composed of ovoid-shaped chondrocyte-like cells. The cells do not lie solitarily, but are aligned in rows of 3-15 cells per row. Collagen fibril bundles are not strictly parallel and much larger than those found in zone 1. Type II collagen is now found within the pericellular matrix of the chondrocytes, with the matrix still made up predominantly of type I collagen. This zone is primarily avascular, and the primary sulfated proteoglycan is aggrecan. The next zone is mineralized fibrocartilage. In this zone, chondrocytes appear more circular and hypertrophic, surrounded by larger pericellular matrix distal from the ACL. Type X collagen, a specific marker for hypertrophic chondrocytes and subsequent mineralization, is detected and found only within this zone. The interface between mineralized fibrocartilage and subjacent bone is characterized by deep inter-digitations. Increasing number of deep inter-digitations is positively correlated to increased resistance to shear and tensile forces during development of rabbit ligament insertions. The last zone is the subchondral bone and the cells present are osteoblasts, osteocytes and osteoclasts. The predominant collagen is type I and fibrocartilage-specific markers such as type II collagen are no longer present.

### Studies in the Process of Tendon-to-Bone-Healing

For bone-patellar tendon-bone grafts, bone-to-bone integration with the aid of interference screws is the primary mechanism facilitating graft fixation. Several groups have examined the process of tendon-to-bone healing.

Blickenstaff et al. (1997) evaluated the histological and biomechanical changes during the healing of a semitendinosus autograft for ACL reconstruction in a rabbit model. Graft integration occurred by the formation of an indirect tendon insertion to bone at 26 weeks. However, large differences in graft strength and stiffness remained between the normal semi-tendinosus tendon and anterior cruciate ligament after 52 weeks of implantation.

In a similar model, Grana et al. (1994) reported that graft integration within the bone tunnel occurs by an intertwining of graft and connective tissue and anchoring of connective tissue to bone by collagenous fibers and bone formation in the tunnels. The collagenous fibers have the appearance of Sharpey's fibers seen in an indirect tendon insertion.

Rodeo et al. (1993) examined tendon-to-bone healing in a canine model by transplanting digital extensor tendon into a bone tunnel within the proximal tibial metaphysis. A layer of cellular, fibrous tissue was found between the tendon and bone, and this fibrous layer matured and reorganized during the healing process. As the tendon integrated with bone through Sharpey's fibers, the strength of the interface increased between the second and the twelfth week after surgery. The progressive increase in strength was correlated with the degree of bone in growth, mineralization, and maturation of the healing tissue.

In most cases, tendon-to-bone healing with and without interference fixation does not result in the complete re-establishment of the normal transition zones of the native ACL-bone insertions. This inability to fully reproduce these structurally and functionally different regions at the junction between graft and bone is detrimental to the ability of the graft to transmit mechanical stress across the graft proper and leads to sites of stress concentration at the junction between soft tissue and bone.

Zonal variations from soft to hard tissue at the interface facilitate a gradual change in stiffness and can prevent build up of stress concentrations at the attachment sites.

The insertion zone is dominated by non-mineralized and mineralized fibrocartilages, which are tissues adept at transmitting compressive loads. Mechanical factors may be responsible for the development and maintenance of the fibrocartilagenous zone found at many of the interfaces between soft tissue and bone. The fibrocartilage zone with its expected gradual increase in stiffness appears less prone to failure.

Benjamin et al. (1991) suggested that the amount of calcified tissue in the insertion may be positively correlated to the force transmitted across the calcified zone.

Using simple histomorphometry techniques, Gao et al. determined that the thickness of the calcified fibrocartilage zone was 0.22±0.7 mm and that this was not statistically different from the tibial insertion zone. While the ligament proper is primarily subjected to tensile and torsional loads, the load profile and stress distribution at the insertion zone is more complex.

Matyas et al. (1995) combined histomorphometry with a finite element model (FEM) to correlate tissue phenotype with stress state at the medial collateral ligament (MCL) femoral insertion zone. The FEM model predicted that when the MCL is under tension, the MCL midsubstance is subjected to tension and the highest principal compressive stress is found at the interface between ligament and bone.

Calcium phosphates have been shown to modulate cell morphology, proliferation and differentiation. Calcium ions can serve as a substrate for Ca²⁺-binding proteins, and modulate the function of cytoskeleton proteins involved in cell shape maintenance.

Gregiore et al. (1987) examined human gingival fibroblasts and osteoblasts and reported that these cells underwent changes in morphology, cellular activity, and proliferation as a function of hydroxyapatite particle sizes. Culture distribution varied from a homogenous confluent monolayer to dense, asymmetric, and multi-layers as particle size varied from less than 5 μm to greater than 50 μm, and proliferation changes correlated with hydroxyapatite particles size.

Cheung et al. (1985) further observed that fibroblast mitosis is stimulated with various types of calcium-containing complexes in a concentration-dependent fashion.

Chondrocytes are also dependent on both calcium and phosphates for their function and matrix mineralization. Wuthier et al. (1993) reported that matrix vesicles in fibrocartilage consist of calcium-acidic phospholipids-phosphate complex, which are formed from actively acquired calcium ions and an elevated cytosolic phosphate concentration.

Phosphate ions have been reported to enhance matrix mineralization without regulation of protein production or cell proliferation, likely because phosphate concentration is often the limiting step in mineralization. It has been demonstrated that human foreskin fibroblasts when grown in micromass cultures and under the stimulation of lactic acid can dedifferentiate into chondrocytes and produce type II collagen.

Cheung et al. (1985) found a direct relationship between β-glycerophosphate concentrations and mineralization by both osteoblasts and fibroblasts. Increased mineralization by ligament fibroblasts is observed with increasing concentration of β-glycerophosphate, a media additive commonly used in osteoblast cultures. These reports strongly suggest the plasticity of the fibroblast response and that the de-differentiation of ligament fibroblasts is a function of mineral content in vitro.

Progressing through the four different zones which make up the native ACL insertion zone, several cell types are identified: ligament fibroblasts, chondrocytes, hypertrophic chondrocytes and osteoblasts, osteoclasts, and osteocytes. The development of in vitro multi-cell type culture systems facilitates the formation of the transition zones.

Goulet et al. (2000) developed a bio-engineered ligament model, where ACL fibroblasts were added to the structure and bone plugs were used to anchor the bioengineered tissue. Fibroblasts isolated from human ACL were grown on bovine type I collagen, and the bony plugs were used to promote the anchoring of the implant within the bone tunnels.

Cooper et al. (2000) and Lu et al. (2001) developed a tissue engineered ACL scaffold using biodegradable polymer fibers braided into a 3-D scaffold. This scaffold has been shown to promote the attachment and growth of rabbit ACL cells in vitro and in vivo. However, no multiphased scaffolds for human ligament-to-bone interface are known.

### SUMMARY OF THE INVENTION

This application describes apparatuses and methods for musculoskeletal tissue engineering. Specifically, graft collar and scaffold apparatuses are provided for promoting fixation of musculoskeletal soft tissue to bone.

This application provides for graft collars comprising biopolymer mesh and/or polymer-fiber mesh for fixing tendon to bone. In one aspect, the graft collar comprises more than one region, wherein the regions can comprise different materials configured to promote integration of and the regeneration of the interfacial region between tendon and bone.

This application also provides for scaffold apparatuses and methods for fixing musculoskeletal soft tissue to bone. The scaffold apparatus is multiphasic, preferably triphasic, and each phase is configured promote growth and proliferation of a different cell and its associated tissue. In one aspect, the scaffold apparatus is triphasic, with phases comprising materials to promote growth and proliferation of fibroblasts, chondroblasts, and osteoblasts. In addition, an apparatus comprising two portions, each of said portion being the scaffold apparatus described above is provided, wherein each of said portion encases one end of a soft tissue graft. Further, a triphasic interference screw is provided.

This application further provides apparatuses and methods for inducing formation of fibrocartilage comprising wrapping a graft collar with polymer-fiber mesh configured to apply compression to the graft collar. In another aspect, the polymer-fiber is applied directly to the graft to apply compression to the graft.

### BRIEF DESCRIPTION OF THE DRAWINGS

FIGS. 1: 1A: A schematic diagram of a graft collar, wherein the graft collar comprises a sheet of biopolymer mesh or polymer-fiber mesh, according to one embodiment.

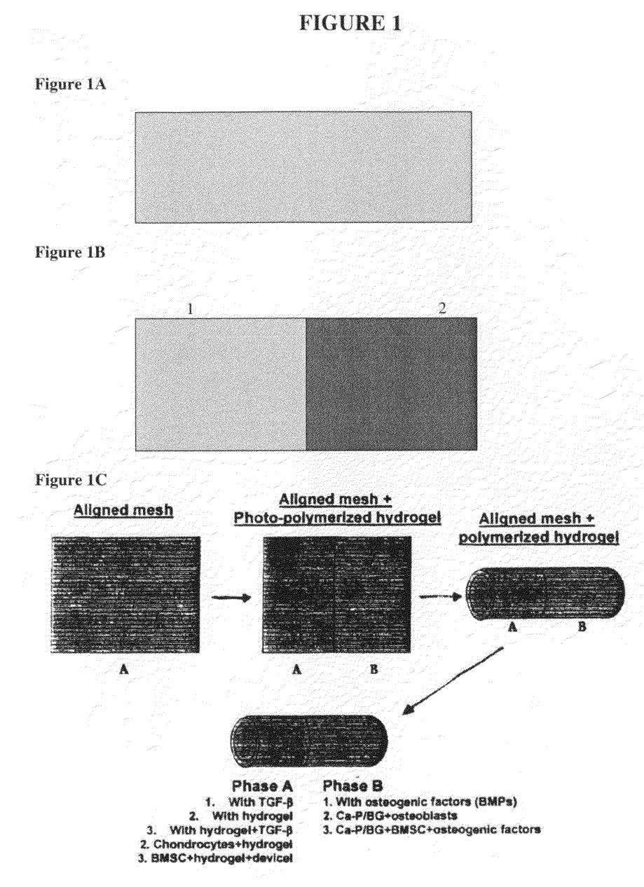

1B-C: A schematic diagram of a graft collar, wherein the graft collar comprises 2 regions wherein (i) region 1 comprises a biopolymer mesh or a polymer-fiber mesh and (ii) region 2 comprises a biopolymer mesh or a polymer-fiber mesh and a hydrogel, according to one embodiment. As indicated, additional substances can be added to regions A and B.

FIGS. 2: 2A: Posterior view of an intact bovine anterior cruciate ligament (ACL) connecting the femur to the tibia (left).

2B: Environmental scanning electron microscope (ESEM) image of transition from ligament (L) to fibrocartilage (FC) to bone (B) at the ACL insertion (upper right).

2C: Histological micrograph of similar ACL to bone interface additionally showing mineralized fibrocartilage (MFC) zone (lower right).

FIGS. 3: 3A: SEM image of Ca—P nodules on BG surface (3 days in SBF). Nodules are ˜1 μm in size initially, and grew as immersion continued (15,000×).

3B: EDXA spectrum of BG surfaces immersed in SBF for 3 days. The relative Ca/P ratio is ≈1.67.

3C shows FTIR spectra of BG immersed in SBF for up to 7 days. Presence of an amorphous Ca—P layer at 1 day, and of a crystalline layer at 3 days.

FIG. 4: 4A-B show environmental SEM images of Bovine ACL insertion site (1 and 2), including a cross section of the ACL-femur insertion site, ACL fiber (L) left, fibrocartilage region (FC) middle, and sectioned bone (B) right (FIG. 4A: 250×; FIG. 4B: 500×).

FIGS. 5: 5A: SEM of the cross section of the femoral insertion zone, 100×.

5B: EDAX of the femoral insertion zone. The peak intensities of Ca, P are higher compared to those in ligament region.

FIG. 6: Shows apparent modulus versus indentation X-position across sample.

FIGS. 7: 7A and B show X-Ray CT scans of discs made of poly-lactide-co-glycolide (PLAGA) 50:50 and bioactive glass (BG) submerged in SBF for 0 days (FIG. 7A) and 28 days; FIG. 7B shows the formation of Ca—P over time.

FIGS. 8: 8A: SEM image.

8B: EDAX of PLAGA-BG immersed in SBF for 14 days.

FIG. 9: Shows osteoblast grown on PLAGA-BG, 3 weeks.

FIG. 10: Shows higher type I collagen type synthesis on PLAGA-BG.

FIGS. 11: 11A: ALZ stain, ACL fibroblasts 14 days, 20×.

11B: ALZ stain, interface, ACL 14 days, 20×.

11C: ALZ stain, osteoblasts, ACL 14 days, 20×.

11D: ALP stain, ACL fibroblasts, 7 days, 32×.

11F: ALP+DAPI stain, co-culture, 7 days, 32×.

11G: ALP stain, osteoblasts, 7 days, 32×.

FIGS. 12: 12A-F show images of multiphase scaffold (Figures A-C) and close-ups of respective sections (D-F).

FIGS. 13: 13A-C show multiphasic scaffold for co-culture of ligament fibroblasts and osteoblasts.

13A and B: images of a sample scaffold.

13C: schematic of scaffold design depicting the three layers.

FIGS. 14: A-D show Micromass co-culture samples after 14 days.

14A: H&E stain.

14B: Alcian blue.

14C: Type I collagen (green).

14D: Type II collagen (green)+Nucleic stain (red).

FIGS. 15: A and B show RT-PCR gel for day 7 micromass samples.

15A: Type X collagen expression.

15B: Type II collagen expression.

(C: control micromass sample; E; experimental co-culture sample)

FIGS. 16: A and B show SEM image of cellular attachment to PLAGA-BG scaffold after 30 minutes:

16A: chondrocyte control (2000×). 16B: co-culture (1500×).

C-E show cellular attachment to PLAGA-BG scaffold:

16C: chondrocyte control, day 1 (500×). 16D: co-culture, day 1 (500×).

16E: co-culture, day 7 (750×).

FIG. 17 show results from Experiment 2:

17A: shows a table of porosimetry data, including intrusion volume, porosity, and pore diameter data.

17B-D: show fluorescence microscopy images (day 28, ×10) for phases A through C, respectively.

17E-F: are images showing extracelluar matrix production for phases B and C, respectively.

FIG. 18: Shows schematic of experimental design for Experiment 3, for in vitro evaluations of human osteoblasts and fibroblasts co-cultured on multi-phased scaffolds.

FIG. 19: Shows results for Experiment 3:

19A: shows a graph which demonstrates cell proliferation in phases A, B, and C during 35 days of human hamstring tendon fibroblast and osteoblast co-culture on multiphased scaffolds.

19B-C: graphically show mechanical testing data for multiphased scaffolds seeded with human hamstring tendon fibroblasts and human osteoblasts over 35 days of culture (n=4).

FIGS. 20: 20A: Schematically shows a method of producing multiphasic scaffolds in experiment 4. First Ethicon PLAGA mesh is cut into small pieces and inserted into a mold. By applying compression force (F) and heating (H) at 150° C. for time (t)=20 hours, the mesh segments are sintered into a mesh scaffold, which is removed from the mold. Next PLAGA microspheres are inserted into the mold, sintered, then removed as a second scaffold. The same process is performed for the PLAGA-BG microspheres. Finally, Phases A and B are joined by solvent evaporation, then all three scaffolds are inserted into the mold and sintered together, forming the final multiphasic scaffold.

20B: shows a schematic of a co-culture experimental design (Experiment 4).

FIG. 21: Shows a table summarizing mercury porosimetry data.

FIGS. 22: 22A-C show graphically scaffold phase thickness and diameters in Experiment 4.

FIGS. 23: A-B: show graphically mechanical testing data for multiphased scaffolds seeded with human hamstring tendon fibroblasts and human osteoblasts over 35 days of culture (n=4). Scaffolds were tested in uniaxial compression. Compressive modulus (A) and yield strength (B) were calculated from the resulting stress-strain curves. Both cell seeded (C) and acellular (AC) scaffolds were examined at days 0, 7, 21 and 35. Scaffold compressive modulus was significantly greater at day 0 than for all subsequent time points and groups (p<0.05).

FIGS. 24: 24A: shows a table illustrating the compositions of polymer solutions tested in experiment 5.

24B: shows a table illustrating drum rotational velocity (rpm) and surface velocity (m/s) for each gear.

FIG. 25: 25A-D show SEMs of electrospun meshes spun at: A) 1^{st} gear, 7.4 m/s; B) 2^{nd} gear, 9.4 m/s; C) 3^{rd} gear, 15 m/s; and D) 4^{th} gear, 20 m/s.

25E-F show scanning electron microscopy (SEM) images of another embodiment of multi-phased scaffold, with 85:15 PLGA electrospun mesh joined with PLGA:BG composite microspheres.

FIG. 26: 26A and 26B: Schematically shows exemplary embodiments of multiphased scaffold as a hamstring tendon graft collar which can be implemented during ACL reconstruction surgery to assist with hamstring tendon-to-bone healing.

FIG. 27: 27A shows an exemplary embodiment of a graft collar (A) comprising a mesh, wherein the fibers of the mesh are aligned substantially parallel to a longitudinal axis of the tendon (B).

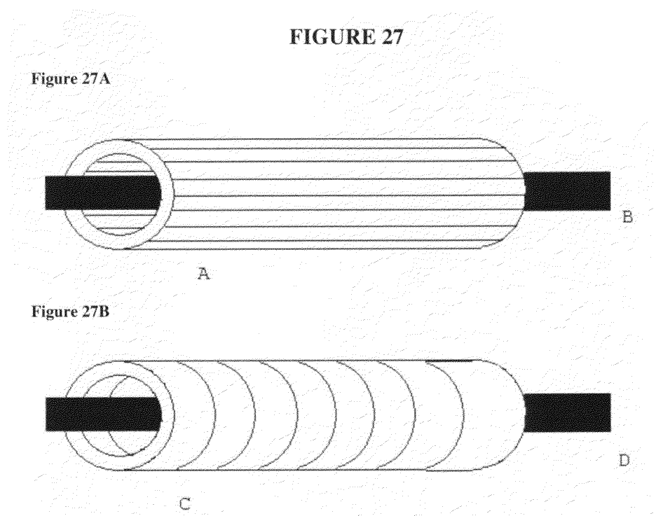

27B shows an exemplary embodiment of a graft collar (C) comprising a mesh, wherein the fibers of the mesh are aligned substantially perpendicular to a longitudinal axis of the tendon (D).

FIG. 28: Characterization of Nanofiber Mesh Contraction. A) As-fabricated nanofiber mesh with preferential fiber alignment at low (left) and high (right) magnification as shown by scanning electron microscopy (low: ×500, high: ×2000). B) Percent contraction of the aligned nanofiber mesh in the direction along (y-axis) and normal to (x-axis) fiber alignment (*p<0.05). Significant mesh contraction was the greatest along the direction of fiber alignment, and contraction stabilized after 24 hours.

FIG. 29: Compression of Graft Collar Scaffold with Nanofiber Mesh. A) Microsphere scaffold wrapped with nanofiber mesh before (top) and after (bottom) 24 hours of mesh contraction. B) Changes in scaffold inner diameter due to compression induced by the nanofiber mesh. While the scaffold-only control swelled (4%), nanofiber mesh contraction induced over 15% decrease in scaffold diameter after 24 hours.

FIG. 30: Compression of Tendon Graft with Nanofiber Mesh.

A) Nanofiber mesh wrapped around a patellar tendon sample before (top, day 0) and after (bottom, day 1) mesh contraction. B) Effects of mesh contraction on tendon matrix organization. After five days of culture, the compressed tendon matrix exhibited greater cell density and is morphologically distinct from the unloaded control. After 14 days, however, no difference was observed between the groups. (H&E, ×10, arrows denote the direction of compressive loading applied by the mesh).

FIG. 31: Compression of Tendon Graft with Graft Collar Scaffold and Nanofiber Mesh. A) Wrapping of the tendon graft with graft collar scaffold and mesh (top) and the tendon graft with mesh+scaffold complex after 24 hours (bottom). B) Effects of compression on tendon matrix organization. Within 24 hours of loading, the tendon matrix no longer exhibits the crimp pattern evident in the unloaded control. In addition, local cell density increased and there is evidence of matrix remodeling, and this organization is maintained after two weeks of static compression. (H&E, ×20, arrows denote the direction of compressive loading applied by the scaffold).

FIG. 32: Effects of Compression on Collagen Organization. Scaffold-induced compression modulated collagen organization. Collagen organization was affected by scaffold-mediated loading at (A) control, Day 1, (B) loaded, Day 1, (C) control, Day 14, and (D) loaded, Day 14. In addition, fiber diameter was smaller in the compressed group. Disruption of the collagen matrix was evident only in the control group after 14 days (Stain, picrosirius red as viewed under polarized light; original magnification, ×20).

FIG. 33: Effects of Compression on Tendon Cellularity and Matrix Composition. A) Cells proliferated in the unloaded group and cell number was significantly higher in the control tendons compared to the compressed tendons after 24 hours of loading (p<0.05). B) Glycosaminoglycan content in the mesh was significantly higher in the compressed group after 24 hours of loading (*p<0.05).

FIG. 34: Effects of Compression on the Expression of Fibrocartilage-Related Markers. Scaffold-induced compression of the tendon graft resulted in significant up-regulation of type II collagen, aggrecan, and TGF-β3 after 24 hours (*p<0.05). All three fibrocartilage interface-related markers increased in the tendon after scaffold-induced compression.

FIG. 35: Manufacturing of the Polymer-Fiber Mesh.

FIG. 36: Graft Collar Scaffold Fabrication.

FIG. 37: Comparison of Scaffold-Induced Dynamic and Static Compression on a Tendon Graft. (A) Experimental Design. (B) Photogrphs of the compressed group and the control group.

FIG. 38: Cross Section of Scaffold+Mesh Complex Applied to Tendon Graft.

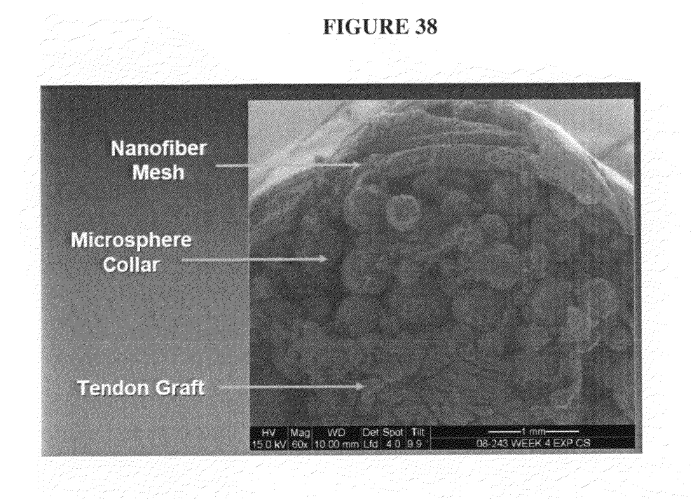

FIG. 39: Effects of Compression on Tendon Graft Matrix Morphology. (A) Control Group. (B) Dynamic Compression Group. (C) Static Compression Group.

FIG. 40: Effects of Compression on Tendon Graft Collagen Fiber Diameter. (A) Control Group. (B) Dynamic Compression Group. (C) Static Compression Group.

FIG. 41: Effects of Compression on Matrix Proteoglycan Content. Greater retention of GAG in the loaded groups.

FIG. 42: Effects of Compression on Cell Number. Cell number constant in the loaded group×106.

FIG. 43: Effects of Compression on Gene Expression. Gene expression for fibrocartilage markers up-regulated in static compressed group over seven days (Collagen, Aggrecan, TGF-β3).

FIG. 44: Effects of Compression on Cell Viability. Cell viability and migration onto the graft collar was observed in the compressed groups.

FIG. 45: Effects of Compression on Tendon Matrix-Preliminary In vivo Study. Little fiber diameter change at day 1 while notable fiber diameter decrease by day 14.

FIG. 46: Effects of wrapping tendon with a PLGA electrospun mesh wherein fibers are either perpendicular or parallel to the longitudinal axis of the tendon. A) Tendons before mesh wrapping: Control Group, no mesh wrapping (left column); Tendons wrapped with mesh having fibers perpendicular to longitudinal axis of tendon (center column); Tendons wrapped with mesh having fibers parallel to longitudinal axis of tendon (right column). B) Tendons 24 hours after mesh wrapping.

FIG. 47: I and II: ACL-to-bone insertion (Trichrome, 5×) III: Biomimetic Triphasic scaffold (Ø 7.5×6.5 mm).

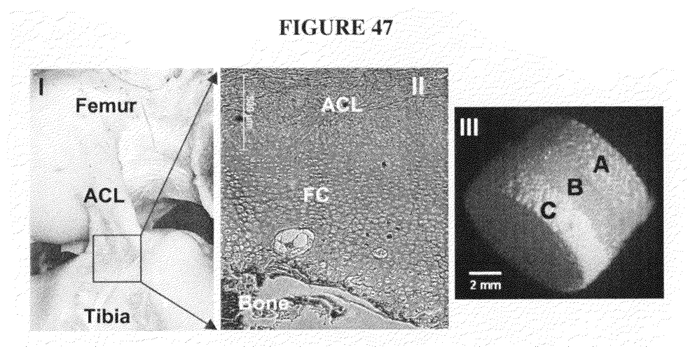

FIG. 48: Clinical application as a bioactive interference screw.

FIG. 49: Schematic summary of experimental approach for Experiment 7.

FIG. 50: I. Multi-phased scaffold design with nanofiber mesh sintered between phases to localize cell seeding. II. Tracking of fibroblasts (Phase A), chondrocytes (Phase B) and osteoblasts (Phase C) on the multi-phased scaffold (Day 1, 10×). Phase specific cell distribution was maintained, which successfully localized fibroblasts (Fb), chondrocytes (CH) and osteoblasts (Ob) on Phase A, B and C, respectively.

FIG. 51: In vivo model. I. Schematic of reconstruction model. II. Reconstruction using flexor tendon graft. III. Bone tunnel formed in the femur and tibia. IV. Microsphere scaffold inserted into the two bone tunnels.

FIG. 52: Experimental design for tracking the three types of implanted cell populations in vivo and determining their presence over a 4-week implantation period.

FIG. 53: Experimental design for interface regeneration on the tri-cultured triphasic scaffold in an intra-articular ACL reconstruction model.

FIG. 54: A schematic view of a triphasic scaffold with degradable cell barrier inserted between adjacent phases.

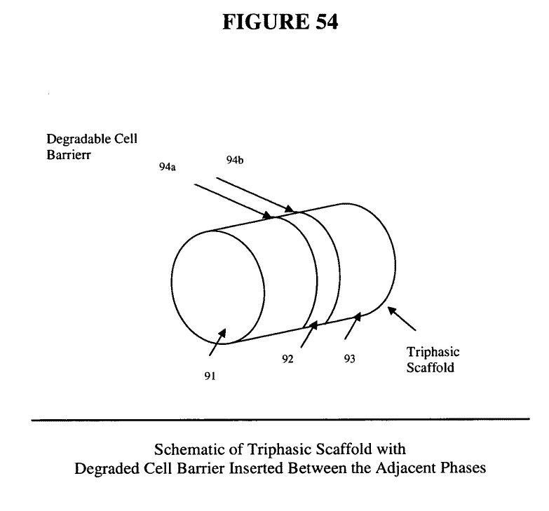

FIG. 55: A schematic view of a triphasic scaffold with degradable cell barrier inserted between adjacent phases.

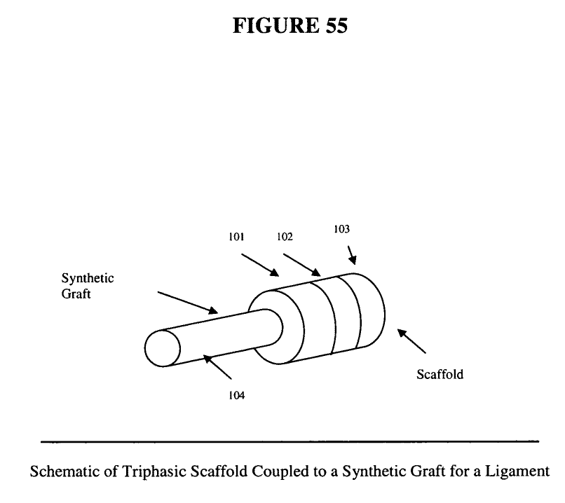

FIG. 56: A schematic view of a scaffold-mesh apparatus coupled with a soft tissue graft.

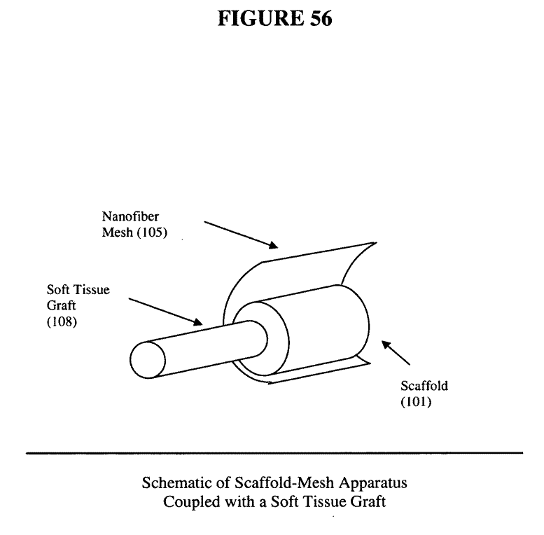

### DETAILED DESCRIPTION OF THE INVENTION

### Terms

In order to facilitate an understanding of the material which follows, one may refer to Freshney, R. Ian. Culture of Animal Cells—A Manual of Basic Technique (New York: Wiley-Liss, 2000) for certain frequently occurring methodologies and/or terms which are described therein.

However, except as otherwise expressly provided herein, each of the following terms, as used in this application, shall have the meaning set forth below.

As used herein, “aligned fibers” shall mean groups of fibers which are oriented along the same directional axis. Examples of aligned fibers include, but are not limited to, groups of parallel fibers.

As used herein, “allogeneic” shall means from the same species. As applied to a graft, allogeneic means that the graft is derived from a material originating from the same species as the subject receiving the graft.

As used herein, “BFGF” shall basic fibroblast growth factor.

As used herein, “bioactive” shall include a quality of a material such that the material has an osteointegrative potential, or in other words the ability to bond with bone. Generally, materials that are bioactive develop an adherent interface with tissues that resist substantial mechanical forces.

As used herein, “biomimetic” shall mean a resemblance of a synthesized material to a substance that occurs naturally in a human body and which is not rejected by (e.g., does not cause an adverse reaction in) the human body.

As used herein, “biopolymer mesh” shall mean any material derived from a biological source. Examples of a biopolymer mesh include, but are not limited to, collagen, chitosan, silk and alginate.

As used herein, “BMP” shall mean bone morphogenic protein.

As used herein, “BMSC” shall mean bone marrow-derived stem cells.

As used herein, “chondrocyte” shall mean a differentiated cell responsible for secretion of extracellular matrix of cartilage.

As used herein, “clamp” shall mean a device which statically compresses the soft tissue graft. The clamp can be made of metal, ceramic, polymers, composites thereof, or other material that can compress a soft tissue graft. The material can be porous, permeable, or degradable.

As used herein, “fibroblast” shall mean a cell of connective tissue, mesodermally derived, that secretes proteins and molecular collagen including fibrillar procollagen, fibronectin and collagenase, from which an extracellular fibrillar matrix of connective tissue may be formed.

As used herein, “functional” shall mean affecting physiological or psychological functions but not organic structure.

As used herein, “GDF” shall mean growth differentiation factor.

As used herein, “glass transition temperature” is the temperature at which, upon cooling, a noncrystalline ceramic or polymer transforms from a supercooled liquid into a rigid glass. The noncrystalline ceramic or polymer may be of multiple form and composition, and may be formed as microspheres. In the context of a sintering process, such as discussed in this application, the polymer chains from adjacent microspheres typically entangle, effectively forming a bond between the microspheres upon cooling. As the polymer is heated above its glass transition temperature, long range polymer chain motion begins.

As used herein, “graft collar” shall mean a device embodying a graft and configured like a collar, that is, having a hollow cylindrical body in a longitudinal direction. A graft collar can be permeable, so the tissue can survive. As indicated by the results of the experiment described in this disclosure, the tissues can survive despite the presence of compression.

As used herein, “graft fixation device” shall mean a device that is useful for affixing a tissue graft to a bone or other body surface, including but not limited to staples, interference (screws with or without washers), press fit EndoButton® devices and Mitek® Anchor devices.

As used herein, “graft” shall mean the device to be implanted during medical grafting, which is a surgical procedure to transplant tissue without a blood supply, including but not limited to soft tissue graft, synthetic grafts, and the like. The graft can be an allograft or an autograft. An “allograft” is tissue taken from one person for transplantation into another. Allografts can include, most commonly, Achilles and tibialis, patellar and quadricepts tendons. An “autograft” or “autologous graft” is a graft comprising tissue taken from the same subject to receive the graft. Graft can also be allogeneic or xenogenic. In one aspect of the present invention, the graft is a soft tissue graft. In another aspect of the present invention, the soft tissue graft is a tendon. In another aspect of the present invention, the graft is a graft for a ligament in a subject, including the ACL. In another aspect of the present invention, the tendon graft can be a bone-patellar tendon-bone (BPTB) graft, a semitendinosus or a hamstring-tendon (HST) graft.

As used herein, “hydrogel” shall mean any colloid in which the particles are in the external or dispersion phase and water is in the internal or dispersed phase. For example, a chondrocyte-embedded agarose hydrogel may be used in some instances. As another example, the hydrogel may be formed from hyaluronic acid, chitosan, alginate, collagen, glycosaminoglycan and polyethylene glycol (degradable and non-degradable), which can be modified to be light-sensitive. It should be appreciated, however, that other biomimetic hydrogels may be used instead.

As used herein, “interference screw” shall mean a type of graft fixation device which anchors a flexible transplant like a tendon or a ligament in an opening in a bone. The screw generally has a screw body, a head at one end of said screw body and a penetrating end at an opposite end of said screw body. The device may be used in, for example, anterior cruciate ligament surgery. The device may be metallic or bioabsorbable and may include, but is not limited to, titanium cannulated interference screws, Poly-L-Lactide (PLLA) interference screws, etc.

As used herein, “lyophilized”, in regards to a graft collar, shall mean a graft collar that has been rapidly frozen and dehydrated.

As used herein, “mechanical loading” shall mean forces applied to a structure or a component which are mechanical in nature, or a mechanical force. In one aspect, the mechanical loading can be compression. In another aspect, the mechanical loading can be tension.

As used herein, “matrix” shall mean a three-dimensional structure fabricated from biomaterials. The biomaterials can be biologically-derived or synthetic.

As used herein, “nanofiber mesh” shall mean a flexible netting of nanofibers, oriented such that at least some of the nanofibers are not parallel to others of the nanofibers.

As used herein, “nanofiber” shall mean fibers with diameters no more than 1000 nanometers.

As used herein, “osteoblast” shall mean a bone-forming cell that is derived from mesenchymal osteoprognitor cells and forms an osseous matrix in which it becomes enclosed as an osteocyte. The term is also used broadly to encompass osteoblast-like, and related, cells, such as osteocytes and osteoclasts.

As used herein, “osteointegrative” shall mean ability to chemically bond to bone.

As used herein, “particle reinforcer” shall mean a composite with a higher strength than the original material.

As used herein, “PDGF” shall mean platelet-derived growth factor.

As used herein, “photopolymerized” shall mean using light (e.g. visible or ultraviolet light) to convert a liquid monomer or macromer into a hydrogel by free radical polymerization.

As used herein, “polymer” shall mean a chemical compound or mixture of compounds formed by polymerization and including repeating structural units. Polymers may be constructed in multiple forms and compositions or combinations of compositions.

As used herein, “porosity” shall mean the ratio of the volume of interstices of a material to a volume of a mass of the material.

As used herein, “PTHrP” shall mean parathyroid hormone-related protein.

As used herein, “sinter” or “sintering” shall mean densification of a particulate polymer compact involving a removal of pores between particles (which may be accompanied by equivalent shrinkage) combined with coalescence and strong bonding between adjacent particles. The particles may include particles of varying size and composition, or a combination of sizes and compositions. For example, sintering a polymer would involve heating the polymer above the glass transition temperature, wherein the polymer chains rearrange and link together to form sintering necks.

As used herein, “soft tissue graft” shall mean a graft which is not synthetic, and can include autologous grafts, syngeneic grafts, allogeneic grafts, and xenogeneic graft.

As used herein, “synthetic” shall mean that the material is not of a human or animal origin.

As used herein, “TGF” shall mean transforming growth factor.

As used herein, “VEGF” shall mean vascular endothelial growth factor.

As used herein, “xenogenic”, shall mean from a different species. As applied to grafts, xenogenic shall mean that the graft is derived from a material originating from a species other than that of the subject receiving the graft.

### EMBODIMENTS

The following exemplary embodiments and experimental details sections are set forth to aid in an understanding of the subject matter of this disclosure but are not intended to, and should not be construed to, limit in any way the subject matter as set forth in the claims which follow thereafter.

This application provides a graft collar for fixing tendon to bone in a subject, wherein said graft collar comprises a sheet of biopolymer mesh or polymer-fiber mesh.

In one embodiment, the biopolymer mesh or polymer-fiber mesh comprises aligned fibers. In another embodiment, the biopolymer mesh or polymer-fiber mesh comprises unaligned fibers. In another embodiment, the graft collar comprises a sheet of biopolymer mesh and the biopolymer mesh is derived from at least one of collagen, chitosan, silk and alginate. In another embodiment, the graft collar comprises a sheet of biopolymer mesh and the biopolymer mesh is allogeneic or xenogenic.

In one embodiment, the graft collar comprises a sheet of polymer-fiber mesh and the polymer-fiber mesh comprises aliphatic polyesters, poly(amino acids), copoly(ether-esters), polyalkylenes oxalates, polyamides, poly(iminocarbonates), polyorthoesters, polyoxaesters, polyamidoesters, poly(ε-caprolactone)s, polyanhydrides, polyarylates, polyphosphazenes, polyhydroxyalkanoates, polysaccharides, biopolymers, poly(lactic-co-glycolic acid), poly(lactide), poly(glycolide) or a blend of two or more of the preceding polymers. In another embodiment, the polymer-fiber mesh comprises at least one of poly(lactide-co-glycolide), poly(lactide) or poly(glycolide).

In one embodiment, the graft collar is sutured around a tendon graft. In another embodiment, the tendon graft is a bone-patellar tendon-bone (BPTB) graft, a patellar tendon graft, a semitendinosus, a hamstring-tendon (HST) graft, a hamstring tendon graft, a quadriceps tendon graft, Achilles graft or tibialis graft. In another embodiment, the tendon graft is an allograft or an autograft.

In one embodiment, the subject is a mammal. In another embodiment, the mammal is a human.

In one embodiment, the graft collar promotes integration of the tendon graft to bone.

In one embodiment, the graft collar includes at least one of the following substances: anti-infectives, antibiotics, bisphosphonate, hormones, analgesics, anti-inflammatory agents, growth factors, angiogenic factors, chemotherapeutic agents, anti-rejection agents, and RGD peptides. In another embodiment, the growth factors are selected from the group consisting of TGFs, BMPs, IGFS, VEGFs and PDGFS. In another embodiment, the TGF is TGF-β. In yet another embodiment, the BMP is BMP-2.

In one embodiment, the graft collar includes one or more of the following types of cells: chondrocytes, osteoblasts, osteoblast-like cells and stem cells. In another embodiment, the graft collar includes at least one of the following: osteogenic agents, osteogenic materials, osteoinductive agents, osteoinductive materials, osteoconductive agents, osteoconductive materials and chemical factors.

In one embodiment, the graft collar promotes regeneration of an interfacial region between tendon and bone.

In one embodiment, the graft collar is lyophilized. In another embodiment, the graft collar is biodegradable. In yet another embodiment, the graft collar is osteointegrative.

This application also provides a graft collar for fixing tendon to bone in a subject, wherein the graft collar comprises: a) a first region comprising a biopolymer mesh or a polymer-fiber mesh and hydrogel; and b) a second region adjoining the first region and comprising a biopolymer mesh or a polymer-fiber mesh, wherein the mesh in the first region and the mesh in the second region are different from each other.

In one embodiment, the subject is a mammal. In another embodiment, the mammal is a human.

In one embodiment, the first region supports the growth and maintenance of an interfacial zone between tendon and bone, and the second region supports the growth and maintenance of bone tissue.

In one embodiment, the graft collar includes at least one of the following substances: anti-infectives, antibiotics, bisphosphonate, hormones, analgesics, antiinflammatory agents, growth factors, angiogenic factors, chemotherapeutic agents, anti-rejections agents, and RGD peptides.

In one embodiment, the hydrogel is photopolymerized, thermoset or chemically cross-linked. In another embodiment, the hydrogel is polyethylene glycol.

In one embodiment, the biopolymer mesh comprises aligned fibers. In another embodiment, the biopolymer mesh comprises unaligned fibers.

In one embodiment, the first region contains TGF. In another embodiments the TGF is TGF-β.

In one embodiment, the first region contains chondrocytes. In another embodiment, the chondrocytes are BMSC-derived.

In one embodiment, the first region contains stem cells. In another embodiment, the stem cells are BMSCs.

In one embodiment, the biopolymer mesh is derived from at least one of collagen, chitosan, silk and alginate.

In one embodiment, the biopolymer mesh is allogeneic or xenogenic.

In one embodiment, the polymer-fiber mesh comprises aliphatic polyesters, poly(amino acids), copoly(ether-esters), polyalkylenes oxalates, polyamides, poly(iminocarbonates), polyorthoesters, polyoxaesters, polyamidoesters, poly(ε-caprolactone)s, polyanhydrides, polyarylates, polyphosphazenes, polyhydroxyalkanoates, polysaccharides, biopolymers, poly(lactic-co-glycolic acid), poly(lactide), poly(glycolide) or a blend of two or more of the preceding polymers. In another embodiment, the polymer comprises at least one of the poly(lactide-co-glycolide), poly(lactide) and poly(glycolide).

In one embodiment, the second region contains at least one of the following growth factors: BMP, IGF, VEGF and PDGF. In another embodiment, the BMP is BMP-2.

In one embodiment, the second region includes osteoblasts and/or osteoblast-like cells. In another embodiment, the osteoblasts and/or osteoblast like cells are BMSC-derived. In another embodiment, the second region includes at least one of the following: osteogenic agents, osteogenic materials, osteoinductive agents, osteoinductive materials, osteoconductive agents, osteoconductive materials and chemical factors. In another embodiment, the second region contains nanoparticles of calcium phosphate. In another embodiment, the calcium phosphate is selected from the group comprising tricalcium phosphate, hydroxyapatite and a combination thereof. In another embodiment, the second region contains nanoparticles of bioglass. In another embodiment, the graft collar is biodegradable. In yet another embodiment, the graft collar is osteointegrative.

In one embodiment, the graft collar is sutured around a tendon graft. In another embodiment, the tendon graft is a bone-patellar tendon-bone (BPTB) graft, a patellar tendon graft, a semitendinosus, a hamstring-tendon (HST) graft, a hamstring tendon graft, a quadriceps tendon graft, Achilles graft or tibialis graft. In another embodiment, the tendon graft is an allograft or an autograft.

This application also provides for a graft collar for fixing tendon to bone in a subject, wherein said graft collar comprises a sheet of mesh comprising fibers aligned substantially perpendicular in relation to a longitudinal axis of said tendon, wherein said mesh applies compression to the graft.

In one embodiment, the mesh comprises a biopolymer. In another embodiment, the mesh comprises a polymer-fiber.

In one embodiment the graft collar comprises a) a first region comprising a mesh and hydrogel; and b) a second region adjoining the first region and comprising a mesh.

This application also provides for a graft collar for fixing tendon to bone in a subject, wherein said graft collar comprises a sheet of mesh comprising fibers aligned substantially parallel in relation to a longitudinal axis of said tendon, wherein said mesh applies lateral tension to the graft.

In one embodiment, the mesh comprises a biopolymer. In another embodiment, the mesh comprises a polymer-fiber.

In one embodiment the graft collar comprises a) a first region comprising a mesh and hydrogel; and b) a second region adjoining the first region and comprising a mesh.

This application also provides a scaffold apparatus for fixing musculoskeletal soft tissue to bone in a subject, said scaffold apparatus comprising two portions, wherein each of the two portions comprising first through third phases, wherein (i) the first phase comprises a material which promotes growth and proliferation of fibroblasts, (ii) the second phase adjacent to the first phase comprises a material which promotes growth and proliferation of chondroblasts, and (iii) the third phase adjacent to the second phase comprises a material which promotes the growth and proliferation of osteoblasts.

In one embodiment, the two portions encase respective portions of a soft tissue graft. In another embodiment, the soft tissue graft is a bone-patellar tendon-bone (BPTB) graft, a semitendinosus or a hamstring-tendon (HST) graft.

In one embodiment, the two portions, in combination, encase the entirety of a soft tissue graft on all sides.

In one embodiment, the soft tissue graft is a bone-patellar tendon-bone (BPTB) graft, a patellar tendon graft, a semitendinosus, a hamstring-tendon (HST) graft, a hamstring tendon graft, a quadriceps tendon graft, Achilles graft or tibialis graft. In another embodiment, the soft tissue graft is an allograft or an autograft.

In one embodiment, a degradable cell barrier is inserted between two adjacent ones of said first through third phases. In another embodiment, the degradable cell barrier comprises a nanofiber mesh. In another embodiment, the nanofiber mesh comprises polylactide-co-glycolide (PLGA). In yet another embodiment, the nanofiber mesh is electrospun.

This application also provides an interference apparatus for affixing soft tissue to bone, comprising the scaffold apparatus described herein.

In one embodiment, the interference apparatus is biomimetic. In another embodiment, the interference apparatus is biodegradable. In yet another embodiment, the interference apparatus is osteointegrative.

This application also provides for a scaffold apparatus for fixing musculoskeletal soft tissue to bone in a subject, said scaffold apparatus comprising (i) a first phase comprising a material which promotes growth and proliferation of fibroblasts, (ii) a second phase adjacent to the first phase comprising a material which promotes growth and proliferation of chondroblasts, and (iii) a third phase adjacent to the second phase comprising a material which promotes the growth and proliferation of osteoblasts, wherein a degradable cell barrier is inserted between two adjacent ones of said first through third phases.

In one embodiment, the degradable cell barrier is a nanofiber mesh. In another embodiment, the nanofiber mesh comprises polylactide-co-glycolide (PLGA). In yet another embodiment, the nanofiber mesh is electrospun.

This application also provides for a scaffold apparatus for fixing musculoskeletal soft tissue to bone in a subject, said scaffold apparatus comprising (i) a first phase comprising a material which promotes growth and proliferation of fibroblasts, (ii) a second phase adjacent to the first phase comprising a material which promotes growth and proliferation of chondroblasts, and (iii) a third phase adjacent to the second phase comprising a material which promotes the growth and proliferation of osteoblasts, wherein said first phase coupled to a soft tissue graft.

In another embodiment, the soft tissue graft is a graft for a ligament of the subject. In another embodiment, the ligament is an anterior cruciate ligament of the subject. In yet another embodiment, the soft tissue graft is a bone-patellar tendon-bone (BPTB) graft, a patellar tendon graft, a semitendinosus, a hamstring-tendon (HST) graft, a hamstring tendon graft, a quadriceps tendon graft, Achilles graft or tibialis graft. In another embodiment, the soft tissue graft is an allograft or an autograft.

In one embodiment, a portion of the scaffold apparatus is configured to be at least partially inserted into a femur of the subject and another portion of the scaffold apparatus is configured to be at least partially inserted into a tibia of the subject.

In one embodiment, the scaffold apparatus is configured to be inserted in a femur of the subject through a tunnel. In another embodiment, the scaffold apparatus is configured to be inserted in a tibia of the subject through a tunnel.

In one embodiment, the first phase is exposed to a joint cavity of the subject. In another embodiment, the second phase is positioned in proximate contact to articular cartilage of the subject. In yet another embodiment, the third phase is encased in bone tissue of the subject.

This application also provides for a scaffold apparatus for fixing musculoskeletal soft tissue to bone in a subject, said scaffold apparatus comprising (i) a graft collar and (ii) a polymer-fiber mesh coupled to the graft collar to apply mechanical loading to the graft collar.

In one embodiment, the mechanical loading is compression. In another embodiment, the mechanical loading is tension.

In one embodiment, the polymer-fiber mesh wraps around the graft collar. In another embodiment, an outer surface of the graft collar is wrapped in its entirety by the polymer-fiber mesh.

In one embodiment, the graft collar is biphasic. In another embodiment, the biphasic graft collar includes a first phase comprising a material which promotes growth and proliferation of chondrocytes, and a second phase adjacent to the first phase comprising a material which promotes the growth and proliferation of osteoblasts.

In one embodiment, the polymer-fiber mesh comprises nanofibers. In another embodiment, the nanofiber mesh comprises polylactide-co-glycolide (PLGA). In yet another embodiment, the nanofiber mesh is electrospun.

In one embodiment, the scaffold apparatus is coupled to a soft tissue graft. In another embodiment, the soft tissue graft is a graft for a ligament of the subject. In another embodiment, the ligament is an anterior cruciate ligament of the subject.

In yet another embodiment, the soft tissue graft is a bone-patellar tendon-bone (BPTB) graft, a patellar tendon graft, a semitendinosus, a hamstring-tendon (HST) graft, a hamstring tendon graft, a quadriceps tendon graft, Achilles graft or tibialis graft. In another embodiment, the soft tissue graft is an allograft or an autograft.

This application also provides for a graft-fixation apparatus comprising the scaffold apparatus described herein. In one embodiment, the graft fixation apparatus is an interference screw.

This application also provides for a scaffold apparatus for fixing musculoskeletal soft tissue to bone, said scaffold apparatus being configured to apply mechanical loading to a soft tissue graft to promote regeneration of a fibrocartilage interface between said soft tissue and said bone. In one embodiment, the soft tissue graft is a bone-patellar tendon-bone (BPTB) graft, a patellar tendon graft, a semitendinosus, a hamstring-tendon (HST) graft, a hamstring tendon graft, a quadriceps tendon graft, Achilles graft or tibialis graft. In another embodiment, the soft tissue graft is an allograft or an autograft.

In one embodiment, the mechanical loading is compression. In another embodiment, the mechanical loading is tension.

In one embodiment, the scaffold apparatus comprises a nanofiber mesh configured to apply said mechanical loading to said soft tissue graft. In another embodiment, said mechanical loading is applied by said scaffold apparatus dynamically or intermittently to said soft tissue graft. In another embodiment, said mechanical loading is applied by said scaffold apparatus statically to promote regeneration of a fibrocartilage interface between said soft tissue and said bone in a subject.

In one embodiment, said scaffold apparatus comprises a material that promotes growth and proliferation of chondroblasts. In another embodiment, said scaffold apparatus comprises first and second phases, wherein (i) the first phase comprises a material that promotes growth and proliferation of chondroblasts, (ii) the second phase adjacent to the first phase comprises a material that promotes growth and proliferation of osteoblasts. In yet another embodiment, said scaffold apparatus comprises first, second and third phases, wherein (i) the first phase comprises a material that promotes growth and proliferation of fibroblasts, (ii) the second phase adjacent to the first phase comprises a material that promotes growth and proliferation of chondroblasts, and (iii) the third phase adjacent to the second phase comprises a material that promotes the growth and proliferation of osteoblasts.

This application also provides for an apparatus for inducing formation of fibrocartilage, said apparatus comprising a graft collar having a hollow central portion along a longitudinal axis, wherein an outer surface of the graft collar is wrapped with a polymer-fiber mesh configured to apply mechanical loading to the graft collar.

In one embodiment, the mechanical loading is compression. In another embodiment, the mechanical loading is tension.

In one embodiment, the graft collar has a cylindrical body. In another embodiment, the graft collar includes a sliced cut parallel to a longitudinal axis. In another embodiment, the outer surface of the graft collar is wrapped in its entirety.

In one embodiment, the polymer-fiber mesh comprises nanofibers. In another embodiment, the nanofibers are aligned.

In another embodiment, the nanofibers are aligned perpendicular to the longitudinal axis of the graft collar.

In another embodiment, the nanofibers are unaligned.

In one embodiment, the graft collar includes at least one of the following substances: anti-infectives, antibiotics, bisphosphonate, hormones, analgesics, anti-inflammatory agents, growth factors, angiogenic factors, chemotherapeutic agents, anti-rejection agents, and RGD peptides. In another embodiment, the growth factors are selected from the group consisting of TGFs, BMPs, IGFs, VEGFs and PDGFs. In another embodiment, the TGF is TGF-β. In yet another embodiment, the BMP is BMP-2.

In one embodiment, the graft collar includes one or more of the following types of cells: chondrocytes, osteoblasts, osteoblast-like cells and stem cells. In another embodiment, the graft collar includes at least one of the following: osteogenic agents, osteogenic materials, osteoinductive agents, osteoinductive materials, osteoconductive agents, osteoconductive materials and chemical factors.

In one embodiment, the polymer-fiber mesh comprises aliphatic polyesters, poly(amino acids), copoly(ether-esters), polyalkylenes oxalates, polyamides, poly(iminocarbonates), polyorthoesters, polyoxaesters, polyamidoesters, poly(ε-caprolactone)ₛ, polyanhydrides, polyarylates, polyphosphazenes, polyhydroxyalkanoates, polysaccharides, biopolymers, poly(lactic-co-glycolic acid), poly(lactide), poly(glycolide) or a blend of two or more of the preceding polymers. In another embodiment, the polymer comprises at least one of the poly(lactic-co-glycolic acid), poly(lactide) and poly(glycolide). In another embodiment, the polymer-fiber mesh is 35% poly(DL-lactide-co-glycolic acid) 85:15, 55% N,N-dimethylformamide, and 10% ethanol. In another embodiment, the polymer-fiber mesh comprises particulate reinforcers. In yet another embodiment, the particulate reinforcers comprise nanoparticles.

In one embodiment, the graft collar is porous. In another embodiment, the graft collar is lyophilized. In another embodiment, the graft collar is biodegradable. In yet another embodiment, the graft collar is osteointegrative.

In one embodiment, the graft collar is composed of microspheres. In another embodiment, the microspheres comprise poly(DL-lactide-co-glycolic acid). In yet another embodiment, the microspheres comprise poly(DL-lactide-co-glycolic acid) and bioactive glass.

In one embodiment, the apparatus further comprises a device which applies static loading to the graft collar. In another embodiment, the device is a clamp.

In one embodiment, the mechanical loading provided by said graft collar is adjusted based on polymer composition. In another embodiment, the mechanical loading provided by said graft collar is adjusted based on fiber composition. In another embodiment, the mechanical loading provided by said graft collar is adjusted based on fiber alignment.

This application also provides for an apparatus described herein, wherein the graft collar comprises: a) first region comprising a polymer-fiber mesh and hydrogel; and b) second region adjoining the first region and comprising polymer microspheres.

In one embodiment, the first region supports the growth and maintenance of an interfacial zone between tendon and bone, and the second region supports the growth and maintenance of bone tissue.

In one embodiment, the graft collar includes at least one of the following substances: anti-infectives, antibiotics, bisphosphonate, hormones, analgesics, anti-inflammatory agents, growth factors, angiogenic factors, chemotherapeutic agents, anti-rejections agents, and RGD peptides.

In one embodiment, the hydrogel is photopolymerized, thermoset or chemically cross-linked. In another embodiment, the hydrogel is polyethylene glycol.

In one embodiment, the polymer-fiber mesh comprises aligned fibers. In another embodiment, the polymer-fiber mesh comprises unaligned fibers.

In one embodiment, the first region contains TGF. In another embodiment, the TGF is TGF-β.

In one embodiment, the first region contains chondrocytes. In another embodiment, the chondrocytes are BMSC-derived.

In one embodiment, the first region contains stem cells. In another embodiment, the stem cells are BMSCs.

In one embodiment, the second region contains at least one of the following growth factors: BMP, IGF, VEGF and PDGF. In another embodiment, the BMP is BMP-2.

In one embodiment, the second region includes osteoblasts and/or osteoblast-like cells. In another embodiment, the osteoblasts and/or osteoblast like cells are BMSC-derived.

In one embodiment, the second region includes at least one of the following: osteogenic agents, osteogenic materials, osteoinductive agents, osteoinductive materials, osteoconductive agents, osteoconductive materials and chemical factors.

In one embodiment, the microspheres comprise poly(DL-lactide-co-glycolic acid). In another embodiment, the microspheres comprise poly(DL-lactide-co-glycolic acid) and bioactive glass.

In one embodiment, the second region contains nanoparticles of calcium phosphate. In another embodiment, the calcium phosphate is selected from the group comprising tricalcium phosphate, hydroxyapatite and a combination thereof. In another embodiment, the second region contains nanoparticles of bioactive glass.

In one embodiment, the graft collar is biodegradable. In another embodiment, the graft collar is osteointegrative.

This application also provides for a method for making a device for inducing formation of fibrocartilage comprising: a) forming a graft collar; and b) wrapping the graft collar prepared in step (a) with a polymer-fiber mesh, to form said device.

In one embodiment, step (a) comprises: (a1) processing a plurality of microspheres; (a2) laying the microspheres processed in step (a) in a mold; and (a3) sintering together the microspheres in the mold above a glass transition temperature.

In one embodiment, the microspheres further comprise bioactive glass. In another embodiment, the polymer-fiber mesh comprises nanofibers. In another embodiment, the polymer-fiber mesh comprises aliphatic polyesters, poly(amino acids), copoly(ether-esters), polyalkylenes oxalates, polyamides, poly(iminocarbonates), polyorthoesters, polyoxaesters, polyamidoesters, poly(ε-caprolactone)s, polyanhydrides, polyarylates, polyphosphazenes, polyhydroxyalkanoates, polysaccharides, biopolymers, poly(lactic-co-glycolic acid), poly(lactide), poly(glycolide) or a blend of two or more of the preceding polymers. In another embodiment, the polymer-fiber mesh comprises at least one of the poly(lactic-co-glycolic acid), poly(lactide) and poly(glycolide). In another embodiment, the polymer-fiber mesh is 35% poly(DL-lactide-co-glycolic acid) 85:15, 55% N,N-dimethylformamide, and 10% ethanol. In another embodiment, the polymer-fiber mesh comprises particulate reinforcers. In yet another embodiment, particulate reinforcers comprise nanoparticles.

In one embodiment, the nanofibers wrapped around the graft collar are perpendicular to the longitudinal axis of the graft collar.

In one embodiment, the method further comprises incubating the polymer-fiber mesh-wrapped graft collar at a suitable temperature, time and humidity to allow sintering of the polymer-fiber mesh to the graft collar.

In one embodiment, the polymer-fiber-mesh-wrapped graft collar is incubated at or around 37° C. and at or around 5% CO₂.

This application also provides for a method for inducing formation of fibrocartilage comprising enclosing a graft within a polymer-fiber mesh-wrapped graft collar configured to apply mechanical loading to the graft. In one embodiment, the graft is a bone-patellar tendon-bone (BPTB) graft, a patellar tendon graft, a semitendinosus, a hamstring-tendon (HST) graft, a hamstring tendon graft, a quadriceps tendon graft, Achilles graft or tibialis graft. In another embodiment, the graft is an allograft or an autograft.

In another embodiment, the mechanical loading is compression.

In one embodiment, the method further comprises: a) replacing the mesh wrapping the graft collar with a new polymer-fiber mesh periodically to apply static compression to the graft. In another embodiment, the method comprises: a) replacing the mesh wrapping the graft collar with a new polymer-fiber mesh every 24 hours to apply static compression to the graft.

In one embodiment, the method further comprises: a) removing the polymer-fiber mesh after a first predetermined period of time, b) allowing the graft to rest for a second predetermined period of time, c) wrapping the graft collar with a new polymer-fiber mesh, and d) repeating steps a)-c), so as to apply dynamic compression to the graft. In another embodiment, the method comprises: a) removing the polymer-fiber mesh after a 24 hours, b) allowing the graft to rest for 24 hours, c) wrapping the graft collar with a new polymer-fiber mesh, and d) repeating steps a)-c), so as to apply dynamic compression to the graft.

In one embodiment, the mechanical loading is tension.

This application also provides for a method for inducing formation of fibrocartilage comprising a graft collar having a hollow central portion along a longitudinal axis wherein an outer surface of the graft collar is clamped by a clamp to apply mechanical loading to the graft collar.

This application also provides for a method for inducing formation of fibrocartilage comprising wrapping a polymer-fiber mesh circumferentially around a graft to apply mechanical loading to the graft. In one embodiment, the graft is a bone-patellar tendon-bone (BPTB) graft, a patellar tendon graft, a semitendinosus, a hamstring-tendon (HST) graft, a hamstring tendon graft, a quadriceps tendon graft, Achilles graft or tibialis graft. In another embodiment, the graft is an allograft or an autograft.

This application also provides for an apparatus for inducing formation of fibrocartilage, said apparatus comprising a graft and a polymer-fiber mesh wrapped circumferentially around the graft to apply mechanical loading to the graft. In one embodiment, the graft is a bone-patellar tendon-bone (BPTB) graft, a patellar tendon graft, a semitendinosus, a hamstring-tendon (HST) graft, a hamstring tendon graft, a quadriceps tendon graft, Achilles graft or tibialis graft. In another embodiment, the graft is an allograft or an autograft.

In another embodiment, the mechanical loading is compression.

In one embodiment, the method further comprises: a) replacing the mesh wrapping the graft with a new polymer-fiber mesh periodically to apply static compression to the graft. In another embodiment, the method comprises: a) replacing the mesh wrapping the graft with a new polymer-fiber mesh every 24 hours to apply static compression to the graft.

In one embodiment, the method further comprises: a) removing the polymer-fiber mesh after a first predetermined period of time, b) allowing the graft to rest for a second predetermined period of time, c) wrapping the graft with a new polymer-fiber mesh, and d) repeating steps a)-c), so as to apply dynamic compression to the graft. In another embodiment, the method comprises: a) removing the polymer-fiber mesh after a 24 hours, b) allowing the graft to rest for 24 hours, c) wrapping the graft with a new polymer-fiber mesh, and d) repeating steps a)-c), so as to apply dynamic compression to the graft.

In one embodiment, the mechanical loading is tension.

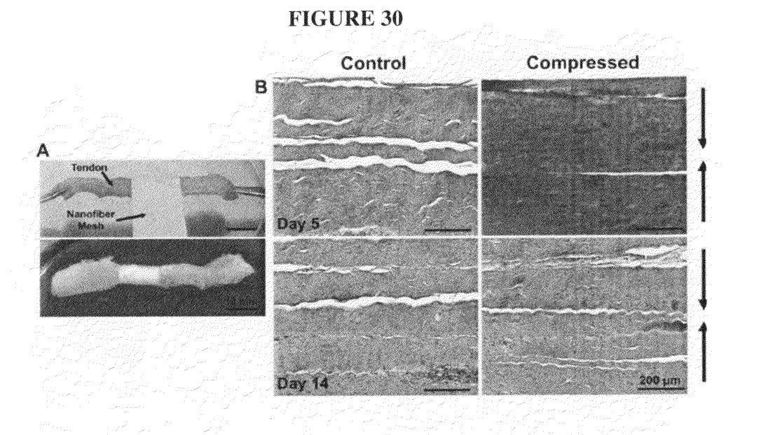

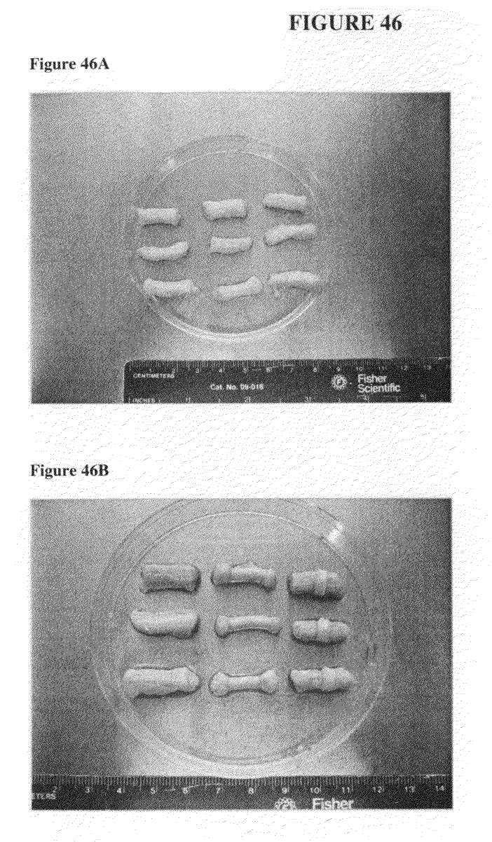

FIGS. 30 and 46B show a polymer-fiber mesh wrapped circumferentially around a tendon graft. FIGS. 30 and 46 are discussed in Experiments 6.1 and 6.2 respectively. FIG. 30A shows nanofiber mesh wrapped circumferentially around a patellar tendon graft and FIG. 30B shows the change in cell phenotype in the tendon resulting from the nanofiber mesh compression. As discussed in Experiment 6.1, the compression resulting from the nanofiber mesh lead to increased cell density. Experiment 6.2 discusses 46B which shows circumferential wrapping of tendon with nanofiber mesh and the resulting change in diameter and length of the tendon.

The specific embodiments and examples described herein are illustrative, and many variations can be introduced on these embodiments and examples without departing from the spirit of the disclosure or from the scope of the appended claims. Elements and/or features of different illustrative embodiments and/or examples may be combined with each other and/or substituted for each other within the scope of this disclosure and appended claims.

Further non-limiting details are described in the following Experimental Details section which is set forth to aid in an understanding of the subject matter but is not intended to, and should not be construed to, limit in any way the claims which follow thereafter.

### EXPERIMENTAL DETAILS

### Experiment 1

### Cell Co-Culture on the Biomimetic Multi-Phased Scaffold

To address the challenge of graft fixation to subchondral bone, a normal and functional interface may be engineered between the ligament and bone. This interface, according to one exemplary embodiment, was developed from the co-culture of osteoblasts and ligament fibroblasts on a multi-phased scaffold system with a gradient of structural and functional properties mimicking those of the native insertion zones to result in the formation of a fibrocartilage-like interfacial zone on the scaffold. Variations in mineral content from the ligament proper to the subchondral bone were examined to identify design parameters significant in the development of the multi-phased scaffold. Mineral content (Ca—P distribution, Ca/P ratio) across the tissue-bone interface was characterized. A multi-phased scaffold with a biomimetic compositional variation of Ca—P was developed and effects of osteoblast-ligament fibroblast co-culture on the development of interfacial zone specific markers (proteoglycan, types II & X collagen) on the scaffold were examined.

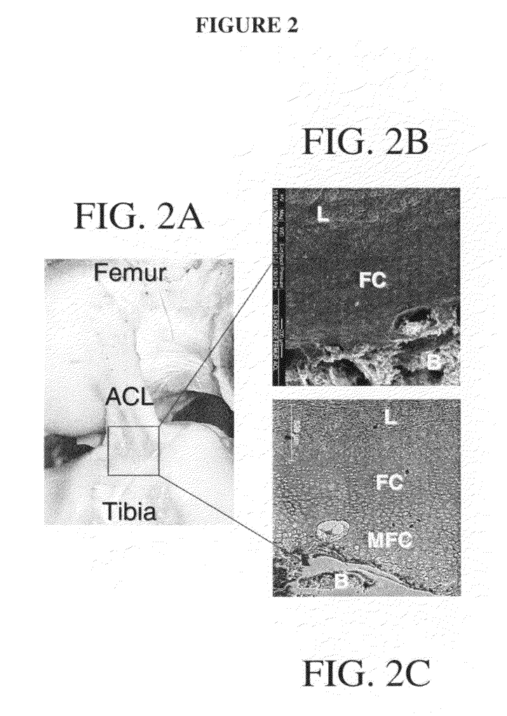

The insertion sites of bovine ACL to bone (FIGS. 2A-2C) were examined by scanning electron microscopy (SEM). Bovine tibial-femoral joints were obtained. The intact ACL and attached insertion sites were excised with a scalpel and transferred to 60 mm tissue culturing dishes filled with Dulbecco's Modified Eagle Medium (DMEM). After isolation, the samples were fixed in neutral formalin overnight, and imaged by environmental SEM (FEI Quanta Environmental SEM) at 15 keV.

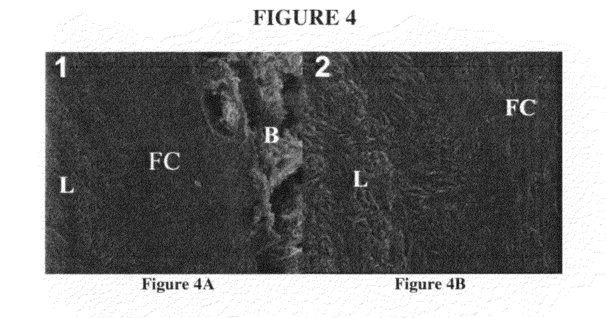

ACL attachment to the femur exhibited an abrupt insertion of the collagen bundle into subchondral bone. When a cross section was imaged (FIGS. 4A-4B), three distinct zones at the insertion site were evident: ligament (L), fibrocartilage (FC), and subchondral bone (B). Sharpey fiber insertion into the fibrocartilage (FIG. 4A) was observed. The bovine interface region spans proximally 600 μm. Examination of the interface using energy dispersive X-ray analysis (EDAX, FEI Company) enable the mineralized and non-mineralized FC zones to be distinguished. A zonal difference in Ca and P content was measured between the ligament proper and the ACL-femoral insertion (see Table I).

TABLE I

| Region | | | | |
|---|---|---|---|---|
| Analyzed | Ca | P | Ca/P Ratio | S |
| Ligament | 1.69 | 2.98 | 0.57 | 3.71 |
| Insertion | 5.13 | 5.93 | 0.87 | 19.50 |

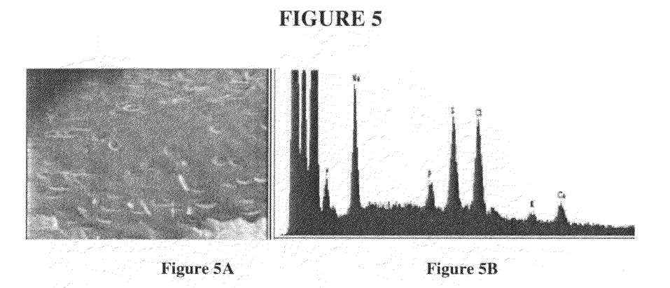

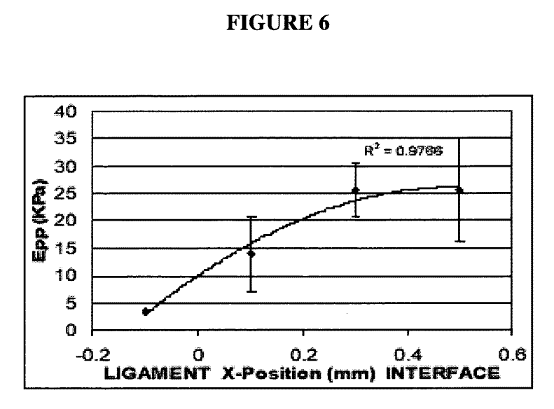

At the insertion zone (FIGS. 5A-5B), higher Ca and P peak intensities were observed, accompanied by an increase in Ca/P ratio as compared to the ligament region. Higher sulfur content due to the presence of sulfated proteoglycans at the FC region was also detected. The zonal difference in Ca—P content was correlated with changes in stiffness across the interface. Nanoindentation measurements were performed using atomic force microscopy (AFM, Digital Instruments). An increasing apparent modulus was measured as the indentation testing position moved from the ligament region into the transition zone (FIG. 6).

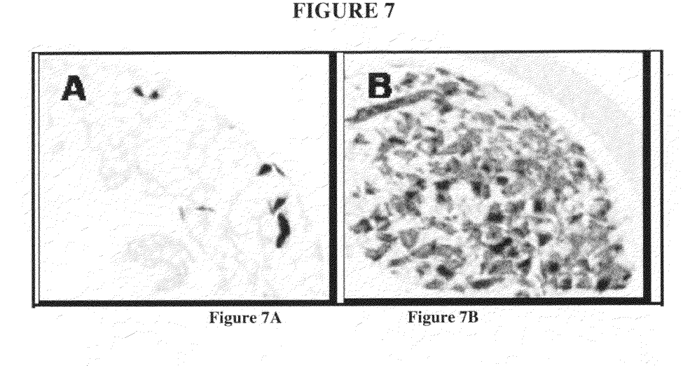

Ca—P distribution on polylactide-co-glycolide (50:50) and 45S5 bioactive glass composite disc (PLAGA-BG) after incubation in a simulated body fluid (SBF) was evaluated using μCT (μCT 20, Scanco Medical, Bassersdorf, Switzerland) following the methods of Lin et al. The sample was loaded into the system, scanned at 20 mm voxel resolution and an integration time of 120 ms. FIGS. 7A and 7B compare the amount of calcified region (dark areas) observed on the PLAGA-BG disc as a function of incubation time in SBF (from day 0 to day 28). Using custom image analysis software, it was determined that at day 0, the mineralized region corresponded to 0.768% of the total disc (quartered) area, and at day 28, the mineralized region corresponded to 12.9% of the total area. Results demonstrate the Ca—P distribution on scaffolds measured by μCT analysis.

The scaffold system developed for the experiments was based on a 3-D composite scaffold of ceramic and biodegradable polymers. A composite system has been developed by combining poly-lactide-co-glycolide (PLAGA) 50:50 and bioactive glass (BG) to engineer a degradable, three-dimensional composite (PLAGA-BG) scaffold with improved mechanical properties. This composite was selected as the bony phase of the multi-phased scaffold as it has unique properties suitable as a bone graft.

A significant feature of the composite was that it was osteointegrative, i.e., able to bond to bone tissue. No such calcium phosphate layer was detected on PLAGA alone, and currently, osteointegration was deemed a significant factor in facilitating the chemical fixation of a biomaterial to bone tissue. A second feature of the scaffold was that the addition of bioactive glass granules to the PLAGA matrix results in a structure with a higher compressive modulus than PLAGA alone.

The compressive properties of the composite approach those of trabecular bone. In addition to being bioactive, the PLAGA-BG lends greater functionality in vivo compared to the PLAGA matrix alone. Moreover, the combination of the two phases serves to neutralize both the acidic byproducts produced during polymer degradation and the alkalinity due to the formation of the calcium phosphate layer. The composite supports the growth and differentiation of human osteoblast-like cells in vitro.

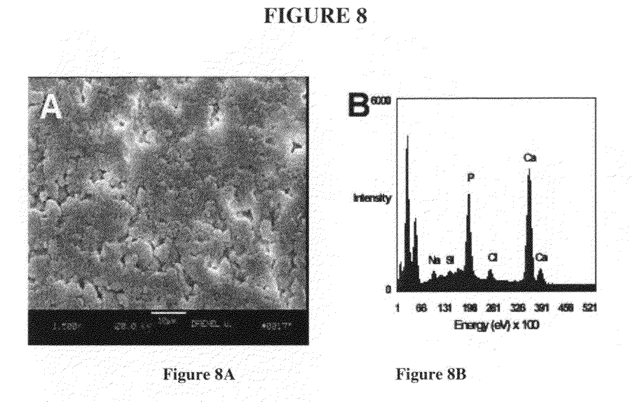

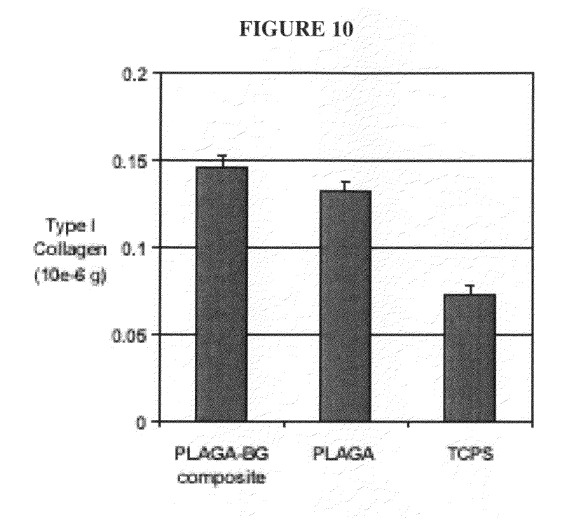

The polymer-bioactive glass composite developed for the experiments was a novel, three-dimensional, polymer-bioactive biodegradable and osteointegrative glass composite scaffold. The morphology, porosity and mechanical properties of the PLAGA-BG construct have been characterized. BG particle reinforcement of the PLAGA structure resulted in an approximately two-fold increase in compressive modulus (p<0.05). PLAGA-BG scaffold formed a surface Ca—P layer when immersed in an electrolyte solution (FIG. 8A), and a surface Ca—P layer was formed. No such layer was detected on PLAGA controls. EDXA spectra confirmed the presence of Ca and P (see FIG. 10B) on the surface. The Ca, P peaks were not evident in the spectra of PLAGA controls.

In vitro formation of a surface Ca—P layer indicates PLAGA-BG composite's osteointegrative potential in vivo. The growth and differentiation of human osteoblast-like cells on the PLAGA-BG scaffolds were also examined. The composite promoted osteoblast-like morphology and stained positive for alkaline phosphatase, and promoted synthesis to a greater extent of Type I collagen synthesis than tissue culture polystyrene controls.

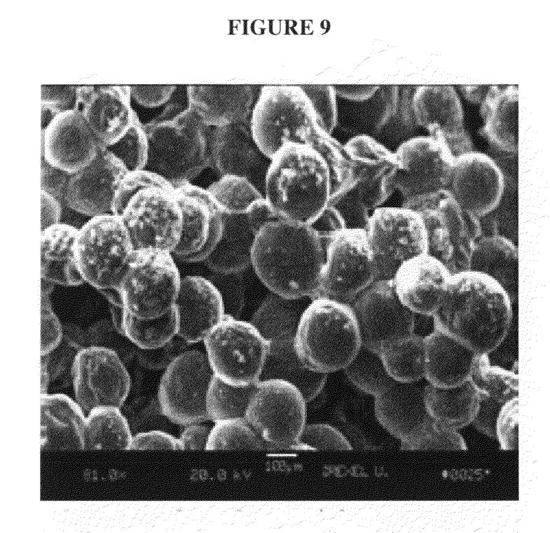

The porous, interconnected network of the scaffold was maintained after 3 weeks of culture (FIG. 9). Mercury porosimetry (Micromeritics Autopore III, Micromeritics, Norcross, Ga.) was used to quantify the porosity, average pore diameter and total surface area of the composite construct. The construct porosity was determined by measuring the volume of mercury infused into the structure during analysis. In addition, the construct (n=6) was tested under compression. BG particle reinforcement of the PLAGA structure resulted in approximately two-fold increase in compressive modulus (see Table II, p<0.05).

TABLE II

| | | Pore | Elastic | Compressive |
|---|---|---|---|---|
| Scaffold | Average | Diameter | Modulus | Strength |
| Type | Porosity | (μm) | (MPa) | (MPa) |
| PLAGA | 31% | 116 | 26.48 ± 3.47 | 0.53 ± 0.07 |
| PLAGA-BG | 43% | 89 | 51.34 ± 6.08 | 0.42 ± 0.05 |

Porosity, pore diameter, and mechanical properties of the scaffold may be variable as a function of microsphere diameter and BG content. The growth and differentiation of human osteoblast-like cells on the PLAGA-BG scaffolds were also examined. The composite supported osteoblast-like morphology and stained positive for alkaline phosphatase.

The porous, interconnected network of the scaffold was maintained after 3 weeks of culture (FIG. 9). The synthesis of type I collagen was found to be the highest on the composite, as compared to the PLAGA and tissue culture polystyrene (TCPS) controls (n=3, p<0.05) (FIG. 10).

The effects of bovine osteoblast and fibroblast co-culture on their individual phenotypes were examined. The cells were isolated using primary explant culture. The co-culture was established by first dividing the surfaces of each well in a multi-well plate into three parallel sections using sterile agarose inserts. ACL cells and osteoblasts were seeded on the left and right surfaces respectively, with the middle section left empty. Cells were seeded at 50,000 cells/section and left to attach for 30 minutes prior to rinsing with PBS. The agarose inserts were removed at day 7, and cell migration into the interface was monitored. Control groups were fibroblasts alone and osteoblasts alone.

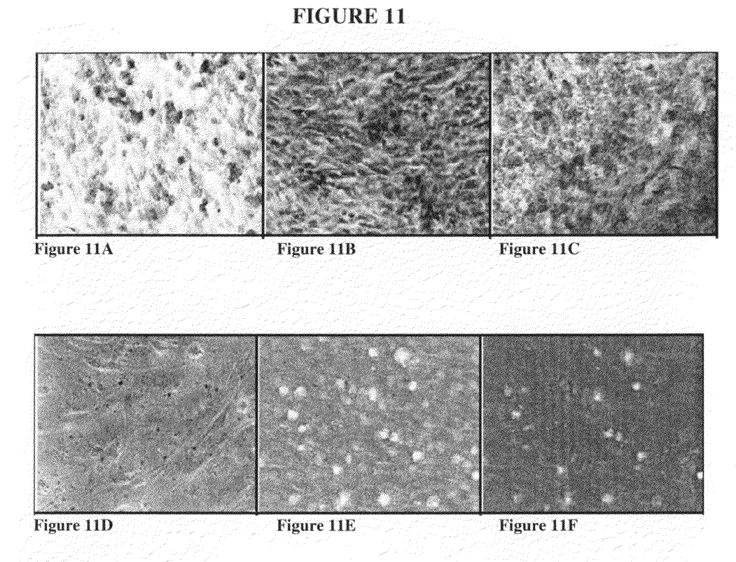

In time, both ACL fibroblasts and osteoblasts proliferated and expanded beyond the initial seeding areas. These cells continued to grow into the interfacial zone, and a contiguous, confluent culture was observed. All three cultures expressed type I collagen over time. The co-culture group expressed type II collagen at day 14, while the control fibroblast did not. Type X collagen was not expressed in these cultures, likely due to the low concentration of b-GP used. Alizarin Red S stain intensity was the highest for the osteoblast control, (FIG. 11C) followed by the co-cultured group (FIG. 11B). Positive ALP staining was also observed for osteoblast control and co-culture groups (see FIGS. 11F and 11E, respectively).

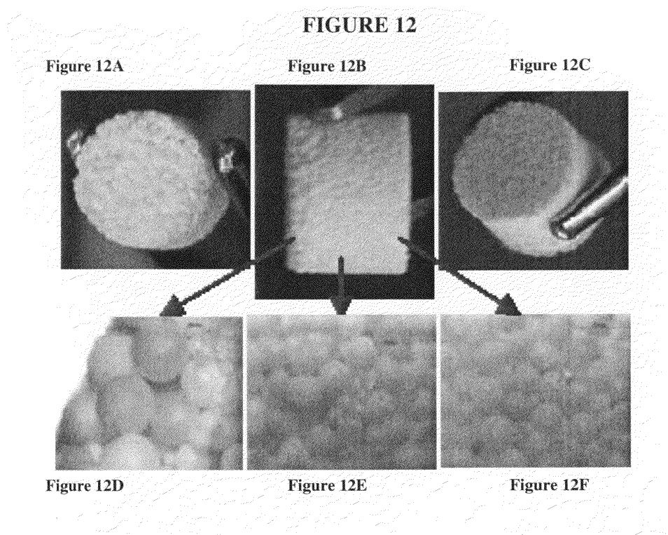

Scaffold of four continuous, graded layers with different sizes of microspheres was formulated (FIGS. 12A-12F). Layered inhomogeneity was pre-designed into the scaffold. Due to differences in packing efficiency between different sizes of microspheres, the porosity of the scaffold decreases from layers of large microsphere to those consisting of small microspheres. PLAGA-BG composite microspheres were produced via the emulsion method. Three layers of PLAGA-BG microspheres of different diameters (250-300, 300-355, 355-500 μm, from top to bottom) were used, shown in FIGS. 12A-12F. Microsphere layers were sintered at 70° C. for 20 hours.

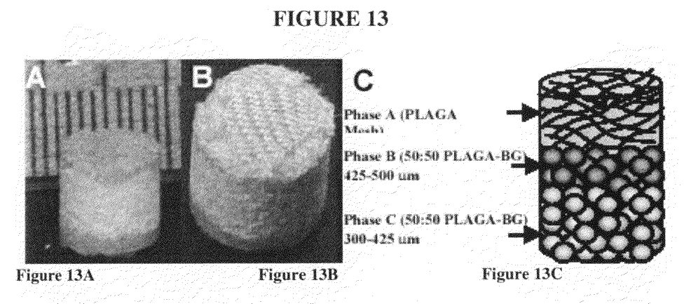

Image analysis confirmed that pore size increased from bottom to top of scaffold. For the growth of ACL fibroblasts on the scaffold, another type of multi-phased scaffold was fabricated using a PLAGA mesh (Ethicon, N.J.) and two layers of PLAGA-BG microspheres. The layers were sintered in three stages in a Teflon mold. First the mesh was cut into small pieces and sintered in the mold for more than 20 hours at 55° C. A layer of PLAGA-BG microspheres with diameter of 425-500 μm was then added to the mold. This layer was sintered for more than 20 hours at 75° C. The final layer consisted of PLAGA-BG microspheres with diameter greater than 300 μm. The scaffolds and three distinct regions were readily observed (FIGS. 13A-13C).

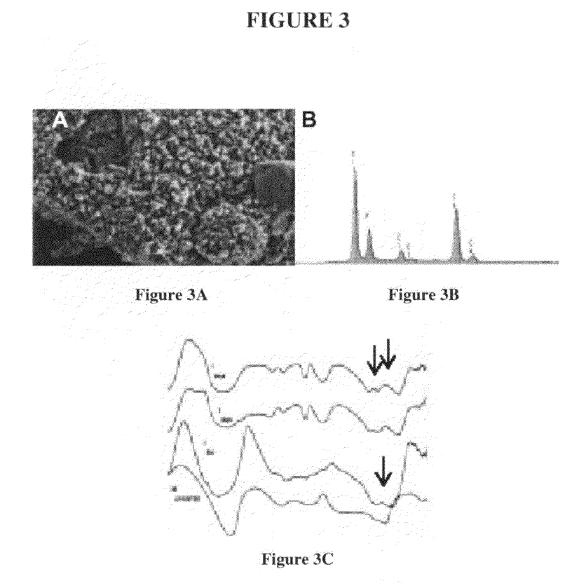

Kinetics of Ca—P layer formation on BG surfaces was related to changes in surface zeta potential in a simulated body fluid (SBF). The chemical and structural changes in BG surface Ca—P layer were characterized using Fourier transform infrared spectroscopy (FTIR), SEM and energy dispersive x-ray analysis (EDXA). FTIR provides information on the degree of crystallinity (amorphous vs. crystalline) of the Ca—P layer formed as well as the functional groups present on BG surface (carbonated Ca—P layer versus non-carbonated, protein adsorption, etc.). FTIR is much more surface sensitive than X-ray diffraction in detecting the Ca—P crystalline structures when the surface layer is only several microns in thickness. SEM combined with EDXA is a powerful tool in relating elemental composition to specific surface morphology and distributions (FIG. 3A-3B). EDXA provides a direct calculation of Ca/P ratio (Ca/P=1.67 for bone mineral and crystalline Ca—P layer) when appropriate standards are used. FTIR, SEM, and EDXA are complimentary techniques which together provide quantitative data on the crystallinity, composition of and functional groups pertaining to the Ca—P layer.

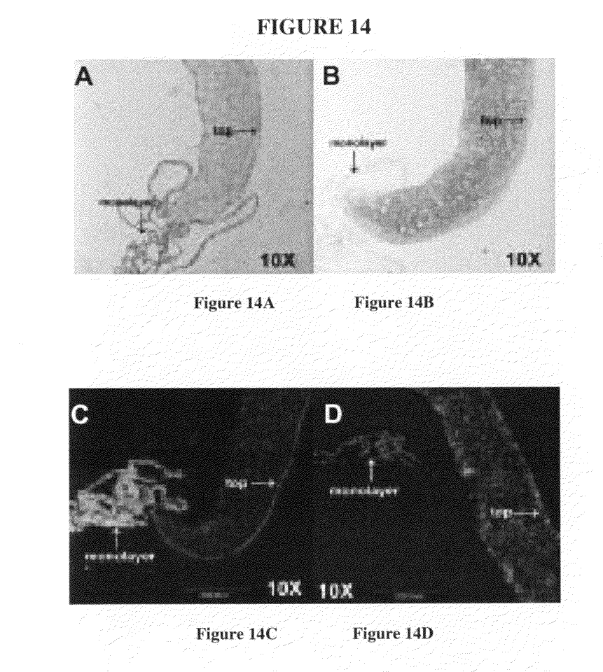

Evaluation of the effects of co-culturing on the growth and phenotypic expression of osteoblasts and chondrocytes. Osteoblasts were seeded directly on high density chondrocyte micromasses. Specific effects of co-culture on the expression of chondrogenic markers were observed primarily at the top surface interaction zone instead of within the micromass. Alcian blue staining (see FIG. 14B) revealed characteristic peri-cellular sulfated GAG deposition by chondrocytes. GAG deposition was found largely within the micromass, instead of at the co-culture zone where elongated osteoblasts and chondrocytes were located. Sulfated GAG was not detected in the predominantly osteoblast monolayer surrounding the micromass. Surface chondrocytes may have dedifferentiated due to co-culturing with osteoblasts. The expression of type I collagen was observed to be distributed mainly on the top surface of the co-cultured mass (FIG. 14C), where osteoblasts were located. Type I was also found at the primarily osteoblastic monolayer surrounding the micromass (see FIG. 14C, left). No type I collagen expression was observed in the chondrocyte-dominated center and bottom surface of the micromass. High expression of type II collagen was observed within the micromass (see FIG. 14D).

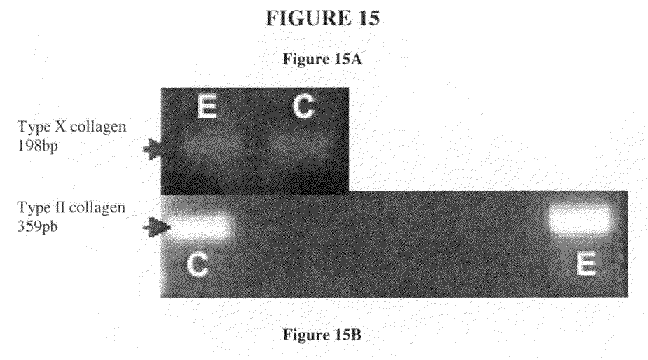

As types I and II collagen were detected at the surface, it is possible that due to co-culture, chondrocytes and osteoblasts were forming an osteochondral-like interface at the surface interaction zone. Alizarin Red (ALZ) staining revealed that there was limited mineralization in the co-cultured group, while the osteoblast control stained increasingly positive for calcium. It is likely that co-culture with chondrocytes may have delayed osteoblast mineralization. Preliminary PCR results (FIGS. 15A and 15B) showed that the 7 day co-culture group expressed types II and X collagen, as detected by RT-PCR.

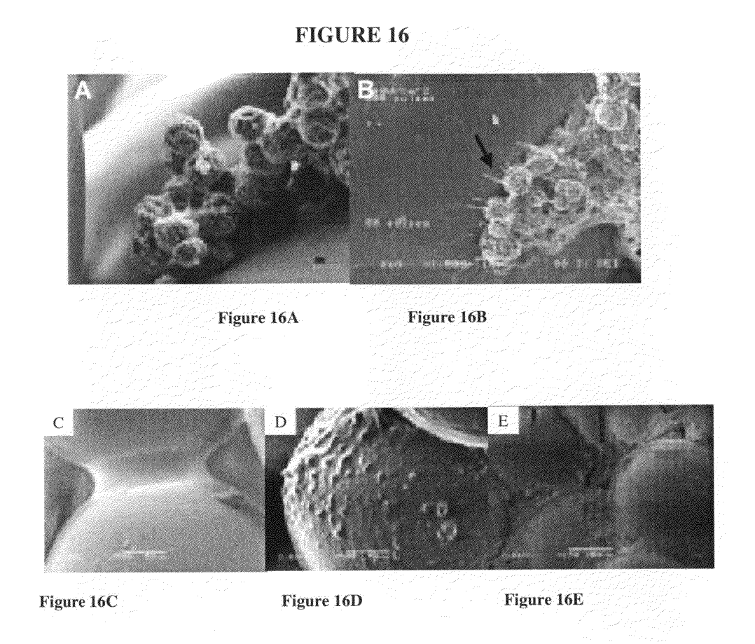

Effects of media additives on the growth and mineralization of osteoblasts and human ACL fibroblasts (HACL) were examined. During mineralization, ALP reacted with β-glycerophosphate (PGP) and the phosphate product was utilized for mineralization. Concentrations (0, 1.0, 3.0, 5.0 mM) effects were examined over time. No significant change in cell number was observed for the [βGP] investigated. At 1.0 mM, a significant difference between 1-day & 7-day samples (p<0.05) was observed. No differences were found between 1.0 mM and 3.0 mM cultures. ALZ stains for the osteoblast cultures were more intense for 3.0 mM than for 1.0 mM. Ectopic mineralization was observed for hACL cultures at 3.0 mM suggesting a potential change in cell phenotype. Interaction of osteoblasts and chondrocytes on a 3-D composite scaffold during co-culture was examined. Scaffolds seeded with only osteoblasts or chondrocytes at the same densities served as controls. Both short-term and long-term co-culture experiments were conducted. Extensive SEM analysis revealed that significant interactions occurred between osteoblasts and chondrocytes during co-culture. Differences in cellular attachment were observed between the chondrocyte control scaffolds and the co-cultured scaffolds. On the co-cultured scaffolds, focal adhesions were evident between the spherical chondrocytes and the surface, indicated by the arrow in FIG. 16B.

No comparable focal adhesions were observed on the chondrocyte controls at the same time point. Chondrocyte morphology changed over time as it assumed a spherical morphology in the first 8 hours, and then spread on the surface of the microspheres (FIG. 16C). The nodules on the surface of the microspheres correspond to the flattened chondrocytes. These nodules were likely chondrocytes instead of calcium phosphate nodules, since calcium phosphate nodules were approximately 1-5 μm in diameter at the culture duration observed and these nodules were ˜10 μm, approximately the diameter of an ovoid cell. After 7 days of culture, the co-culture group exhibited extensive matrix production (FIG. 16E) and expansion on the scaffold.

Examination of the ACL-bone interface confirmed existence of a mineral gradient across the insertion zone and correlation to changes in material properties. Multi-phased scaffolds with controlled morphology and porosity were fabricated. The osteochondral graft developed from co-culture on PLAGA-BG and hydrogel scaffold supported growth of multiple matrix zones with varied GAG and mineral content. BMSCs differentiated into ligament fibroblast and produced a functional extracellular matrix when cultured with growth factors on a fiber-based scaffold. Mineral content, distribution, and chemistry at the interface and on the scaffold were quantifiable using a complimentary set of surface analysis techniques (FTIR, SEM, EDAX, μCT). Electron microscopy examination of the ACL-bone interface revealed insertion zone including three different regions: ligament, fibrocartilage-like zone, and bone. Co-culture of osteoblasts and ligament fibroblasts on 2-D and 3-D scaffolds resulted in changes in cell morphology and phenotype. Type X collagen, an interfacial zone marker, was expressed during co-culture. Multi-phased scaffold with layered morphology and inhomogeneous properties were designed and fabricated. FTIR, SEM and EDXA are complimentary techniques which collectively provided qualitative and quantitative information on the Ca—P layer and composition of the calcium phosphate surface.

These experiments illustrate, in relevant part, the interaction between osteoblasts and chondrocytes on a scaffold apparatus during co-culture.

### Experiment 2

### Design and Testing of a Triphasic and Continuous Scaffold with Controlled Heterogeneity Seeded with Bovine or Human Cells

The degree of graft integration is a significant factor governing clinical success and it is believed that interface regeneration significantly improves the long term outcome. The approach of this set of experiments was to regenerate the ACL-bone interface through biomimetic scaffold design and the co-culture of osteoblasts and fibroblasts. The interface exhibits varying cellular, chemical, and mechanical properties across the tissue zones, which can be explored as scaffold design parameters. This study describes the design and testing of a multi-phased, continuous scaffold with controlled heterogeneity for the formation of multiple tissues. The continuous scaffold consists of three phases: Phase A for soft tissue, Phase C for bone, and Phase B for interface development. Each phase was designed with optimal composition and geometry suitable for the tissue type to be regenerated. Fibroblasts were seeded on Phase A and osteoblasts were seeded on Phase C, and the interactions of osteoblasts and fibroblasts (ACL and hamstring tendon) during co-cultures on the scaffolds were examined in vitro.

Phases A, B and C consist of poly(lactide-co-glycolide) (PLAGA, 10: 90) woven mesh, PLAGA (85:15) microspheres, and PLAGA (85:15)/Bioactive Glass (45S5,BG) composite microspheres, respectively. The microspheres were formed via a double emulsion method, and the continuous multi-phased scaffolds were formed by sintering above the polymer T_{g}. Scaffold porosity and pore diameter were determined by porosimetry (Micromeritics, n=3) and the samples were tested under uniaxial compression (MTS 810, n=5) at 1.3 mm/min up to 5% strain with 10 N preload.

Bovine and human osteoblasts (bOB and hOB), and bovine ACL fibroblasts (bFB) and human hamstring tendon fibroblasts (hFB) were obtained through explant culture. In experiment I, bOB and bFB (5×10⁵ cells each/scaffold) were co-cultured on the scaffold, and cell viability, attachment, migration and growth were evaluated by electron and fluorescence microscopy. The bOB were pre-labeled with CM-DiI, and both cell types were labeled with calcein AM (Molecular Probes) prior to imaging. Matrix production and mineralization were determined by histology. After ascertaining cell viability on the scaffolds, a more extensive experiment using hOB and hFB was conducted in which cell proliferation and differentiation and above analyses were investigated. The mechanical properties of the seeded scaffolds were also measured as a function of culture time.

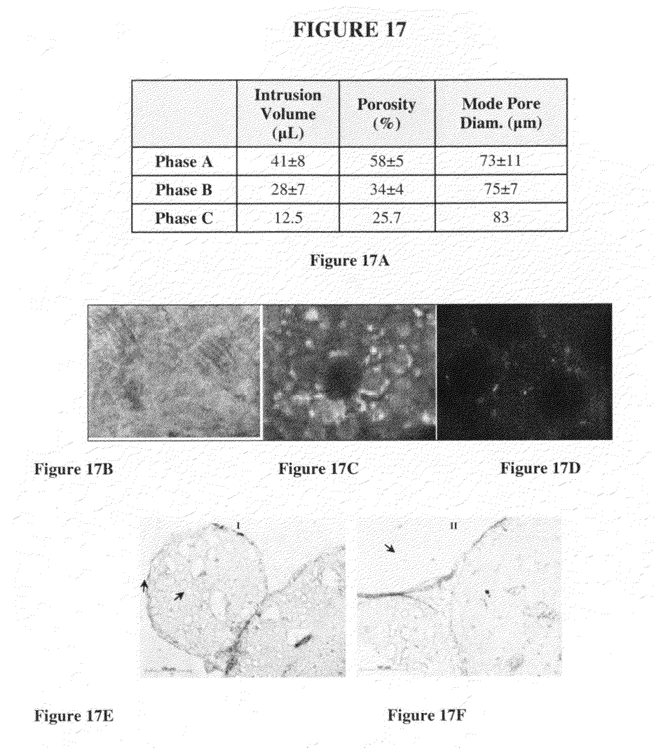

Compression testing of scaffolds indicated an average modulus of 120120 MPa and yield strength of 2.3 MPa. The intrusion volume, porosity and pore diameter data are summarized in the table shown in FIG. 17A.

The fibroblasts and osteoblasts were localized primarily at the two ends of the scaffolds after initial seeding, with few cells found in Phase B. After 28 days, both cell types migrated into Phase B (FIG. 17C), and extensive cell growth was observed in Phases A and C (FIGS. 17B-17D).

Extensive collagen-rich matrix production was found throughout the three phases at day 28 (FIGS. 17E-17F).

The biomimetic, multi-phased scaffolds supported the growth and ECM production of both osteoblasts and fibroblasts. After 28 days of culture, collagen production was evident in all three phases and mineralized matrix was found in the bone and interface regions. Osteoblast and fibroblast interaction at the interface (Phase B) suggests that these cells may play a significant role in the development of a functional insertion site.

These findings demonstrate, in relevant part, that this novel scaffold is capable of simultaneously supporting the growth of multiple cell types and can be used as a model system to regenerate the soft tissue to bone interface.

### Experiment 3

### In Vitro Evaluation of Human Osteooblasts and Fibroblasts Co-Cultured on Multi-Phased Scaffold

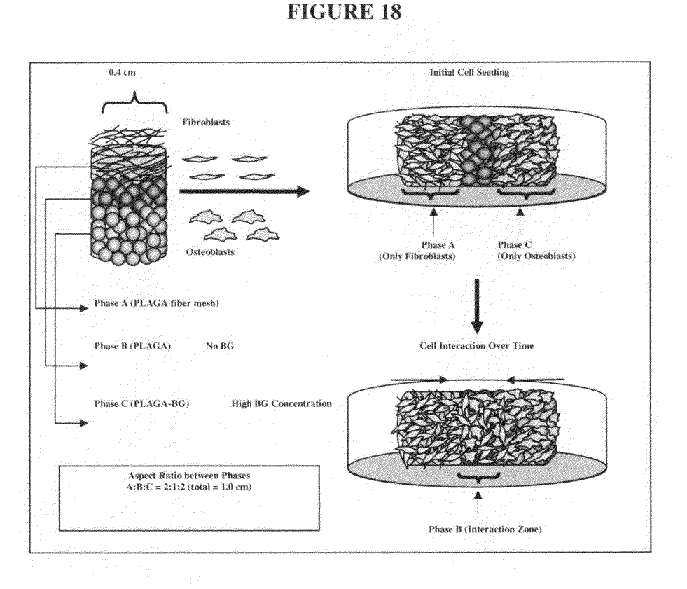

This set of experiments was directed to in vitro evaluations of human osteoblasts and fibroblasts co-cultured on multi-phased scaffolds. A schematic of the experimental design for the in vitro study is shown in FIG. 18. Phase A (mesh) was seeded with human hamstring tendon fibroblast cell suspension. Phase C was seeded with osteoblasts. Cell interaction in the interfacial Phase B was monitored over time. Acellular scaffolds served as controls.

Cell proliferation in Phases A, B, and C during 35 days of human hamstring tendon fibroblast and osteoblast co-culture on multiphased scaffolds is shown in FIG. 18. A general trend of increasing cell number was observed in each phase over time. Data demonstrates that all three phases of the scaffold support cellular viability and proliferation. A higher number of cells were seeded on phase A due to its inherently larger surface area compared to phase C.

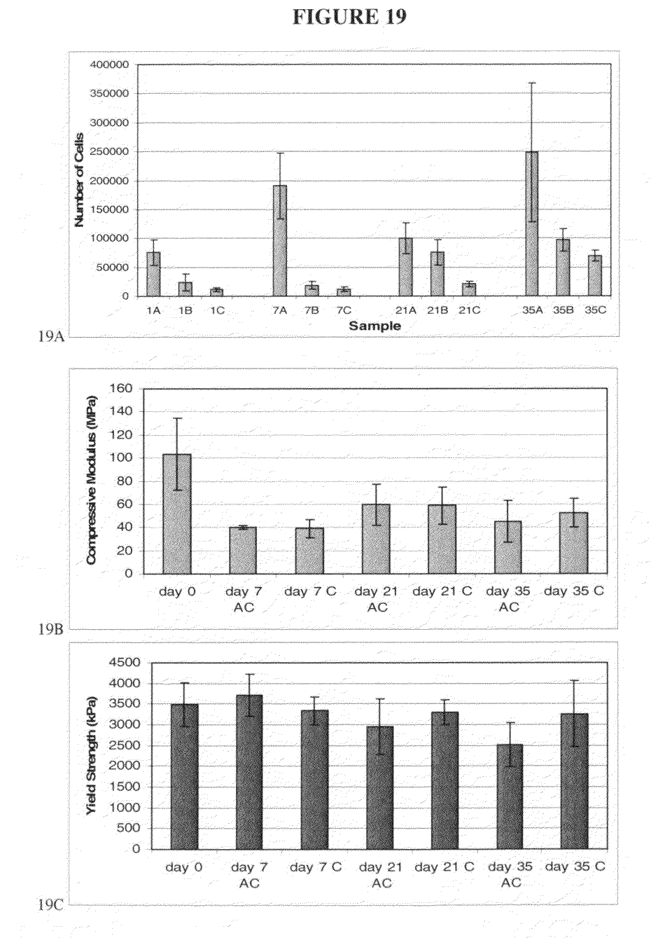

Mechanical testing data for multiphased scaffolds seeded with human hamstring tendon fibroblasts and human osteoblasts over 35 days of culture (n=4) is graphically shown in FIGS. 19B-19C. Scaffolds were tested in uniaxial compression.

Compressive modulus (FIG. 19B) and yield strength (FIG. 19C) were calculated from the resulting stress-strain curves. Both cell seeded (C) and acellular (AC) scaffolds were examined at days 0, 7, 21, and 35.

Compared to the acellular controls, the cell seeded scaffolds degraded slower and better maintained their structural integrity over time. The yield strength of the acellular scaffold decreased over 35 days, while the seeded scaffolds maintained its yield strength.

These experiments, in relevant part, illustrate the interaction between osteoblasts and human hamstring tendon fibroblasts on a multi-phase scaffold.

### Experiment 4

### In Vitro Evaluation of Co-Culture of Human Hamstring Tendon Fibroblasts and Travecular Bone Osteoblasts on Multi-Phased Scaffold

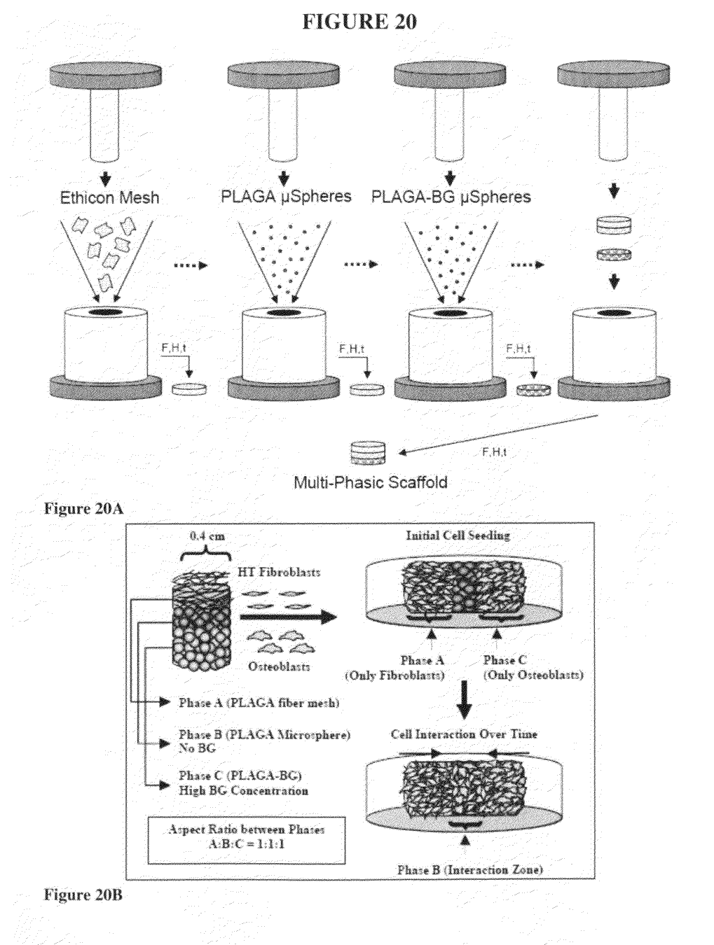

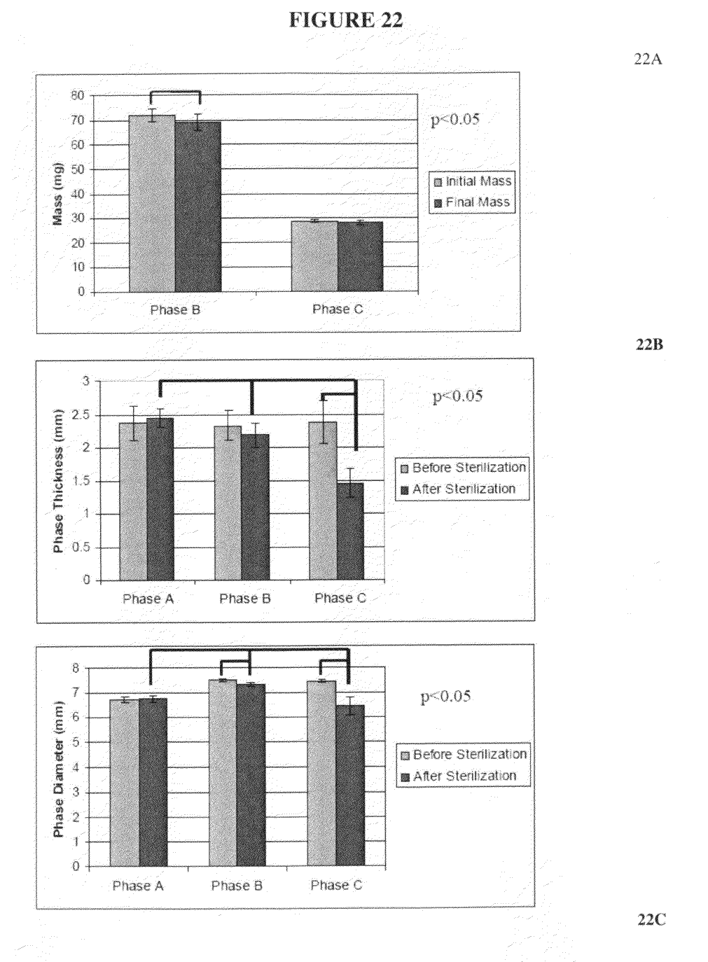

The scaffold designed for this study consisted of three phases and were fabricated in four stages (FIG. 20A). First, Phase A was formed from polyglactin 10:90 PLGA mesh sheets (Vicryl VKML, Ethicon). Mesh sheets were cut into small segments (approximately 5 mm×5 mm) and inserted into cylindrical molds (7.44 mm diameter). Molds were heated to 150° C. for 20 hours to sinter the segments together to form a cylindrical mesh scaffold. The next phase (Phase B) consisted of 100% 85:15-poly(DL-lactide-co-glycolide) (PLAGA, Alkermes Medisorb, M, <<123.6 kDa) microspheres formed by a water/oil/water emulsion. Briefly, Ig PLAGA was dissolved in 10 mL methylene chloride (EM Science, Gibbstown, N.J.) and poured into a mixing 1% PVA surfactant solution (Sigma Chemicals, St. Louis, Mo.). Microspheres were mixed for 4 hours, recovered by filtration, allowed to dry in a fume hood overnight, then vacuum desiccated for 24 hours. To form the PLAGA microsphere phase, −0.075 g microspheres were inserted into the same molds as used previously, and sintered at 55° C. for 5 hours. The last phase (Phase C) consisted of composite microspheres formed from an 80:20 ratio of PLAGA and 45S5 bioactive glass (BG, MO-SCI Corporation, Rolla, Md.). Again, microspheres were formed by emulsion, except with 0.25 g bioactive glass suspended in a solution of 1 g PLAGA in 10 mL methylene chloride. Microspheres (28-30 mg/scaffold) were sintered in the same molds at 55° C. for five hours. After all three phases were sintered separately, Phases A and B were joined by methylene chloride solvent evaporation, and then sintered to Phase C for 10 hours at 55° C. in the same molds. Subsequently, scaffolds were sterilized with ethylene oxide. Final scaffold dimensions are detailed in FIGS. 22B-22C.

Human osteoblast-like cells and hamstring tendon fibroblasts were obtained from explant culture of tissue isolated from humerus trabecular bone and hamstring tendon respectively. Trabecular bone was rinsed with PBS, then cultured in Dulbecco's Modified Eagle's Medium (DMEM, Mediatech, Herndon, Va., USA) supplemented with 10% fetal bovine serum, 1% non essential amino acids, and 1% penicillin/streptomycin (Mediatech, Herndon, Va.), and incubated at 37° C. in a 5% CO₂ incubator to allow for cell migration. Hamstring tendon obtained from excess tissue utilized for hamstring tendon ACL reconstruction autografts was minced and cultured in similarly supplemented DMEM. The first migrations of cells were discarded to obtain a more uniform cell distribution. Second migration, passage 2 osteoblast-like cells and second and third migration, passage 5 hamstring tendon fibroblasts were utilized for the co-culture experiment.

Scaffold dimensions were measured prior to cell seeding and before and after EtO sterilization. Phase thickness was calculated by image analysis, while phase diameter was determined using a digital caliper. Scaffold porosity and pore diameter (Phases A and B: n=3; Phase C: n=1) were determined by mercury porosimetry (Micromeritics Autopore III and Autopore IV 9500, Micromeritics, Norcross, Ga.). The porosity data were utilized to determine cell seeding densities and cell suspension volumes for Phases A and C, with the volumes calculated such that fibroblasts suspension remains in Phase A and osteoblasts suspension in Phase C.

Hamstring tendon fibroblasts were seeded at a density of 250,000 cells/scaffold in a volume of 40.7 μL/scaffold on Phase A (FIG. 20B). After allowing the fibroblasts to attach to the scaffolds for 20 minutes, the scaffolds were rotated upside down so that Phase C faced upwards. Subsequently, 75,000 osteoblast-like cells were seeded per scaffold in a volume of 12.5 μL. After allowing the osteoblasts to attach to the scaffold for 20 minutes, the scaffolds were covered with DMEM supplemented with 10% FBS, 1% NEAA, and 1% penicillin/streptomycin, and incubated at 37° C. and 5% CO₂. Ascorbic acid at a concentration of 20 μg/mL was added beginning at day 7. Media was exchanged every two days. Scaffolds were cultured in 6-well plates and covered with 7 mL of supplemented media per scaffold to minimize pH fluctuations due to rapid poly(glycolic acid) degradation.

Cell attachment, migration, and proliferation on the multi-phased scaffolds were examined using SEM (5 kV, JEOL 5600LV) at days 7, 21, and 35. The scaffolds were fixed with Karnovsky's glutaraldehyde fixative, and stored at 4° C. for 24 hours. The samples were then rinsed with Hank's buffered salt solution two times, and serially dehydrated with ethanol. Cross-sections of the scaffold phases were mounted on an aluminum post and gold-coated prior to analysis.

Extracellular matrix production and mineralization were determined via histology at day 35. Scaffolds were rinsed two times with room temperature PBS. The scaffolds were then covered with 10% neutral buffered formalin and stored at 4 degrees C. Samples were plastic embedded using a modification of a procedure developed by Erben. The scaffolds were first suspended in 2% agarose (low gelling temperature, cell culture grade, Sigma, St. Louis, Mo.), then serially dehydrated with ethanol and cleared with xylene substitute (Surgipath, Sub-X, Richmond, Ill.). Following dehydration, samples were embedded in poly(methyl methacrylate) (Polysciences, Inc., Warrington, Pa.) and sectioned into 10 μm slices. The scaffold sections were stained with either hematoxylin and eosin, von Kossa or Picrosirius Red stains and imaged with light microscopy.

At days 1, 7, 21, and 35, scaffolds were rinsed twice with PBS and subsequently the three phases were separated. Each phase was then stored in 0.1% Triton-X at −80° C. Cellular proliferation in each phase was determined by means of PicoGreen DNA quantitation assay.

In addition, cellular phenotype for mineralization was evaluated using a quantitative alkaline phosphatase (ALP) assay.

At days 0, 7, 21, and 35, seeded and acellular scaffolds were tested under uniaxial compression (MTS 810, n=4). The crosshead speed was 1.3 mm/min, and the scaffolds were compressed up to 35-40% strain. A 10 N preload was applied prior to testing. The effects of scaffold degradation and extracellular matrix production on scaffold compressive modulus were examined.

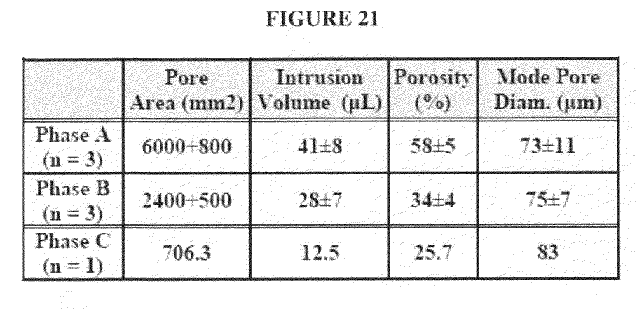

Mercury porosimetry data for each phase are summarized in the table shown in FIG. 21. Scaffold dimensions are shown in FIGS. 22B-22C. The thickness of Phase C decreased significantly (p<0.05) due to contraction during the EtO sterilization (FIG. 22B). In addition, the thicknesses of all phases were significantly different from each other after sterilization. Scaffold diameters also varied due to contraction during sintering, in the case of Phase A, and contraction of Phase C during sterilization. The diameters of Phases B and C decreased significantly after sterilization, and the diameters of all phases were significantly different from each other after sterilization (p<0.05). During the scaffold fabrication process, microspheres are lost between weighing and filling the molds. This loss is mainly due to static charge accumulation in one or more of the microspheres, weighing paper, or mold, which prevents a small percentage of the microspheres from entering the molds. PLAGA-BG microspheres for Phase C generally experience a 2.1±1.4% loss in mass, while the PLAGA microspheres for Phase B suffer a loss of 4.0±1.8% (FIG. 22A). Composite microspheres are generally more statically charged than the PLAGA microspheres; however, the stainless steel mold, used more often for the composite microspheres, dissipates charge buildup more readily than the PTFE mold, which is used more often for the PLAGA microspheres, possibly explaining why there is a significant loss for Phase B (p<0.05). Mesh for Phase A is not susceptible to this loss.

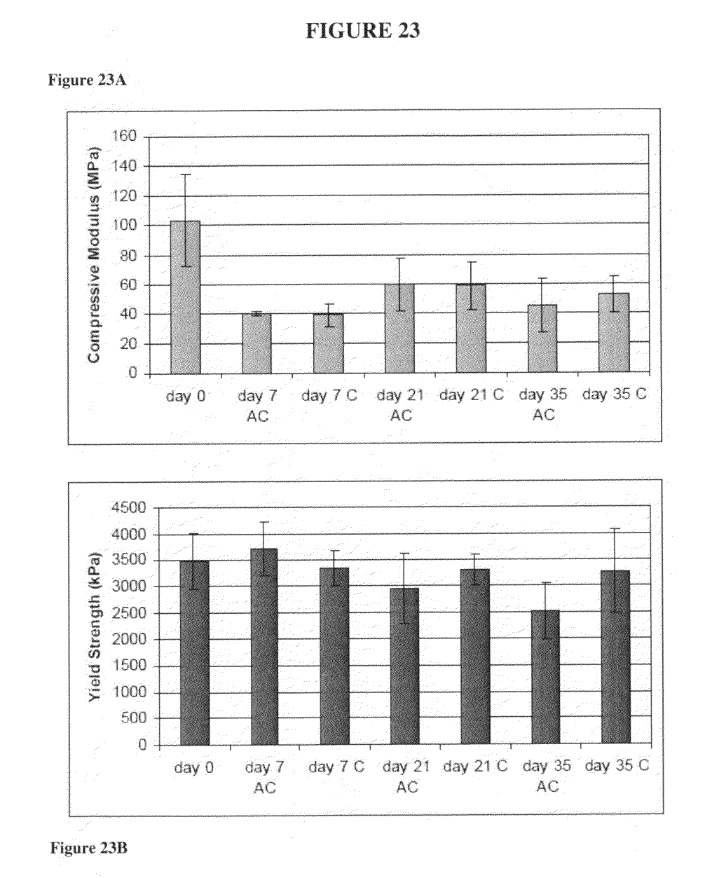

Compressive modulus and yield strength were obtained for seeded and acellular control scaffolds at days 0, 7, 21, and 35 of culture. A rapid decrease in compressive modulus was observed following day 0, possibly due to rapid initial polymer degradation. By day 35, the seeded scaffolds exhibited a greater compressive modulus (FIG. 23A) and yield strength (FIG. 23B), possibly due to cellular extracellular matrix and mineralization compensating loss of scaffold strength due to polymer degradation.

In this experiment, the cell types were switched from bovine ACL fibroblasts and trabecular bone osteoblast-like cells to human hamstring tendon fibroblasts and trabecular bone osteoblasts due to the increased clinical relevance of these new cell types. This experiment aimed to acquire quantitative data about cell proliferation and migration throughout the three phases, as well as cellular alkaline phosphatase activity in each phase of the scaffold.

Based on the previous experiment performed with bovine cells, it is apparent that the biomimetic, multi-phased scaffolds support the growth and ECM production of both osteoblasts and fibroblasts. After 28 days of culture, collagen production was evident in all three phases and mineralized matrix was found in the bone and interface regions. Osteoblast and fibroblast interaction at the interface (Phase B) suggests that these cells may play a significant role in the development of a functional insertion site.

These findings demonstrate that this novel scaffold is capable of simultaneously supporting the growth of multiple cell types and can be used as a model system to regenerate the soft tissue to bone interface.

### Experiment 5

### Multi-Phased Scaffold with Electrospun Plaga Mesh

The objective of the set of experiments was to incorporate electrospun PLAGA meshes into the multi-phased scaffold design, substituting the Ethicon mesh phase, and allowing the entire scaffold to be made in-house.

Electrospinning, short for electrostatic spinning, is a relatively new term that describes a principle first discovered in the first half of the 20 century (see, for example, U.S. Pat. Nos. 1,975,504, 2,160,962, 2,187,306, 2,323,025 and 2,349,950 to Formhals, the entire contents of which are incorporated herein by reference). Electrostatic spinning involves the fabrication of fibers by applying a high electric potential to a polymer solution. The material to be electrospun, or dissolved into a solution in the case of polymers, is loaded into a syringe or spoon, and a high potential is applied between the solution and a grounded substrate. As the potential is increased, the electrostatic force applied to the polymer solution overcomes surface tension, distorting the solution droplet into a Taylor cone from which a jet of solution is ejected toward the grounded plate. The jet splays into randomly oriented fibers, assuming that the solution has a high cohesive strength, linked to polymer chain molecular weight, to prevent droplets from forming instead of fibers in a process known as electrospraying. These fibers have diameters ranging from nanometer scale to greater than 1 μm and are deposited onto the grounded substrate or onto objects inserted into the electric field forming a non-woven mesh. Mesh characteristics can be customized by altering electrospinning parameters. For example, fiber diameter and morphology can be altered, including the formation of beads along the fibers, by controlling applied voltage and polymer solution surface tension and viscosity. Also, fiber orientation can be controlled by rotating the grounded substrate. This high degree of customizability and ability to use many different materials, such as biodegradable polymers and silks, grant this fabrication method a high potential in the development of materials for biomedical application. Management of fiber diameter allows surface area to be controlled, and polymers with different degradation rates can be combined in various ratios to control fiber degradation, both of which are significant in drug delivery applications. Also, controlling the orientation of fiber deposition grants a degree of control over cell attachment and migration. Moreover, the ability to electrospin fiber meshes onto non-metal objects placed in the electric field enables the fabrication of multiphasic scaffold systems.

Here, in order to obtain precise parameters for the mesh fibers, including fiber diameter, morphology, and alignment, the effects of processing parameters on fiber characteristics were studied. A variable-speed rotating drum was designed and constructed to serve as a substrate for aligned fibers, and rheological experiments were performed on the polymer solutions to determine the effect of polymer concentration on solution viscosity and the subsequent effect of solution viscosity on fiber diameter and morphology.

In addition to determining the speed of each gear, the effect of each speed on fiber alignment was determined qualitatively. A 30% v/v PLAGA solution was prepared with 60% dimethylformamide and 10% ethanol, and this solution was electrospun onto the rotating drum at each of the four speed settings. The resulting meshes were examined by scanning electron microscopy (JEOL 5600LV).

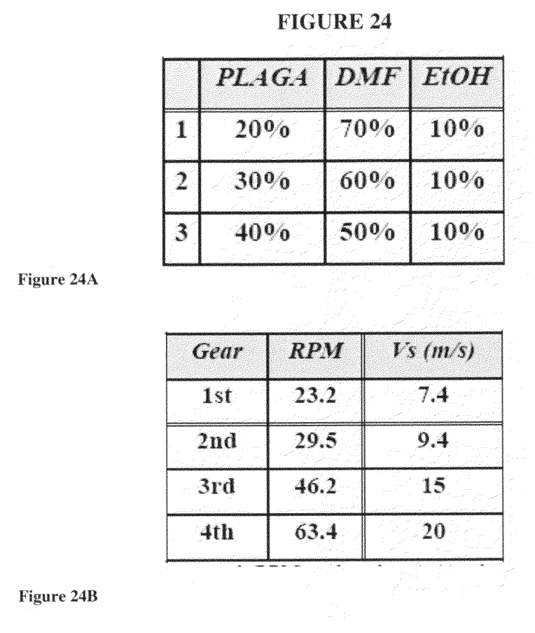

The relationship between polymer concentration (Alkermes 85:15 PLAGA) and solution viscosity was determine by means of a rheological study. Three concentrations of polymer were tested −20%, 30%, and 40% v/v—in dimethylformamide (DMF) and ethanol. The composition of each solution is listed in the table shown in FIG. 24A. Solutions were analyzed using an Advanced Rheometer AR 200Ot. There was variability in the viscosity measurements (n=1) at different strain rates due to the evaporation of solvent during testing. The geometry used for the viscosity measurements was a 25 mm stainless steel disc. A solvent trap was not used since it is not designed to fit with this geometry and a prior trial using the solvent trap with another geometry resulted in poor results, possibly because water from the solvent trap seal interacted with the polymer solution. Additional trials can use a solvent trap to obtain consistent and reliable values for viscosity. For the present study, averages were taken of the viscosity measurements taken at strain rates tested after the equipment had equilibrated. As a result, there are standard deviations for the viscosity measurements even with an n of 1.

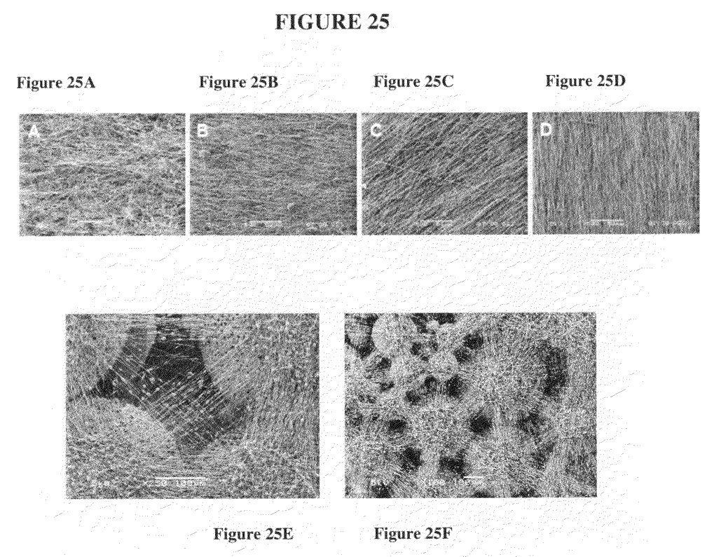

The surface velocity of the rotating drum was seen to increase with increased pulley positions from gear 1 to gear 4 (see the table shown in FIG. 24B). The degree of fiber alignment increased with increasing drum velocity, as seen in the SEMs of each mesh (FIG. 25A-25D).

It was found that (as expected) the degree of fiber orientation increased with increasing drum rotational velocity. The image was analyzed and a histogram of fiber angles was generated against the horizontal axis of the image at regular interval across the image. Thus, the degree of alignment of the fibers can be quantified. It is desirable to control the degree of fiber alignment in the electrospun meshes so that the extracellular environment found at the interface can be mimicked. By producing biomimetic scaffolds, it was intended to direct cell growth to reproduce the tissue inhomogeneity found at the native ACL insertions. In addition to controlling the fiber alignment, it is desirable to control fiber diameter and morphology. It was previously determined that substituting 10% of the DMF in the polymer solutions with ethanol reduces the surface tension of the solution and results in a significant reduction in the number of beads formed along the fibers when electrospinning PLAGA. This effect was also observed by Fong et al., who reduced the number of beads in electrospun poly(ethylene oxide) (PEO) meshes by the addition of ethanol. Surface tension of the polymer solution acts to form spheres during the electrospinning process. By reducing the solution surface tension, the formation of spheres is less favorable and straighter fibers result. Fong et al. also determined that the addition of ethanol increased the viscosity of the PEO:water solutions, which also favors the formation of straight fibers, and results in increased fiber diameter. Deitzel et al. also have demonstrated a relationship between PEO: water solution viscosity and fiber diameter, with fiber diameter increasing with increasing viscosity according to a power law. A relationship between solution viscosity and concentration of polymer can be determined in order to understand how PLAGA: N,W-DMF viscosity affects fiber diameter and morphology. The effect of solution viscosity on fiber diameter and morphology can be determined by spinning the various solutions and examining the resulting meshes by SEM. Other variables can affect the fiber parameters. By changing the percentage of polymer, the surface tensions of the polymer solutions also change in addition to the viscosity. Therefore, in addition to testing the viscosities of each solution, the surface tension of each solution are measured. It is desirable to keep all variables constant except for viscosity in order to truly determine the effect of solution viscosity on fiber characteristics. However, the interrelation of many of the electrospinning parameters complicates the process.

A PLAGA mesh was electrospun directly onto a microsphere scaffold. This is one way to incorporate the mesh. In addition, the scaffolds can be secured to the drum and aligned fibers electrospin directly onto the scaffolds. However, because of the high rotational velocities, it is difficult to secure the scaffolds and prevent them from flying off the drum when it begins rotating. Alternatively, aligned fiber meshes can simply be spun separately, and then later sintered to the microsphere scaffolds. For example, aligned fiber meshes can be electrospun onto aluminum foil, then wrapped around a rod with multiple mesh sheets sintered together to obtain a hollow cylinder of aligned fibers.

These experiments illustrate one possible method, i.e. electrospinning, for the production of aligned fibers for use in PLAGA scaffold apparatuses.

FIGS. 25E and 25F show scanning electron microscopy (SEM) images of another embodiment of multiphased scaffold, with 85:15 PLGA electrospun mesh joined with PLAGA:BG composite microspheres.

### Experiment 6.1

### Mechanical Compression of Tendon Graft or Graft Collar

As discussed supra, ACL inserts into subchondral bone through a fibrocartilage interface, which can be subdivided into non-mineralized and mineralized regions (Cooper, 1970; Messner, 1997; Niyibizi, 1996; Peterson, 1999; Sagarriga, 1996; Wang, 2006; Wei, 1996).

The principal function of this complex interface is to minimize stress concentrations and to facilitate load transfer between two distinct tissue types (Benjamin, 1986; Matyas, 1995; Moffat, 2006; Spalazzi, 2006; Woo, 1988; Woo, 1983). While the mechanism governing the formation of the fibrocartilage interface is not well understood, it has been postulated that fibrocartilage forms due to metaplasia of tendon or ligament (Gao, 1996). Nawata et al. (Nawata, 2002) examined the development of ACL insertions in a rodent model, and reported that insertion site fibrochondrocytes are derived from ligament fibroblasts. Benjamin and Ralphs observed that the amount of non-mineralized fibrocartilage at an enthesis may be related to the degree of motion at the tendon- or ligament-to-bone interface, suggesting that mechanical signaling is responsible for fibroblast differentiation into fibrochondrocytes and subsequent fibrocartilage formation (Benjamin, 1998). These reports, coupled with the observation that fibrocartilage develops in regions where the tendon is subjected to compression (Benjamin, 1986; Vogel, 1986), collectively suggest that compressive loading is necessary for inducing tendon fibroblast trans-differentiation and fibrocartilage formation on tendon grafts.

Vogel et al. (Koob, 1992; Malaviya, 2000; Perez-Castro, 1999; Robbins, 1997; Vogel, 1996) have conducted studies investigating the effects of compressive loading in fibrocartilage formation in flexor tendons, and have reported that compression may induce metaplasia of tendinous matrix to fibrocartilage. The presence of this fibrocartilage region is believed to enable tendons to resist compressive loading via the accumulation of proteoglycans (Vogel, 1989). For example, while gene expression for aggrecan was absent in the wrap-around region of fetal and neonatal bovine deep flexor tendons, the proteoglycan was strongly expressed in mature animals, suggesting post-natal remodeling of fibrocartilage with physiological loading (Perez-Castro, 1999). In addition, anterior translocation of the rabbit flexor digitorum profundus tendon to remove compressive loading led to a decrease in the size of the fibrocartilage region, breakdown of the collagen fiber network, and lower matrix glycosaminoglycan content (Malaviya, 2000). Moreover, in vitro dynamic compressive loading of fibrocartilaginous regions of bovine deep flexor tendon resulted in increased expression of aggrecan, biglycan, and versican after 72 hours (Robbins, 1997). Recently, Wang et al. observed that the fibrocartilage interface of the ACL-to-bone insertion undergoes significant structural changes during post-natal development, after the onset of physiological loading (Wang, 2006). Spalazzi et al. demonstrated the existence of compressive strains at the ACL insertion sites when the tibiofemoral joint is loaded in tension (Spalazzi, 2006). These results suggest that a dynamic loading regime, as when an animal walks, may also be necessary for the metaplasia of tendon. Therefore, compressive loading may be important for fibrocartilage formation and remodeling at the ACL-to-bone enthesis.

To address the challenge of achieving biological fixation of soft tissue-based ACL reconstruction grafts, functional methods to regenerate an anatomic fibrocartilage transition on tendon grafts need to be developed. To this end, a novel scaffold system was designed which can directly apply compressive mechanical loading to tendon grafts. Specifically, this scaffold system combines a degradable graft collar (Lu, 2003; Spalazzi, 2006) with nanofiber meshes fabricated from poly(lactic-co-glycolic acid) (PLGA) (Li, 2002, Moffat, 2007). It is anticipated that with the inherent contraction of the nanofiber meshes (Moffat, 2007; Zong, 2003), this biphasic scaffold system can be used to apply compressive mechanical loading to tendon grafts and induce fibrocartilage formation. To test this hypothesis, experiments were conducted with a number of objectives, beginning with the characterization of the contractile properties of the nanofiber mesh as well as the mesh+graft collar scaffold complex. The second objective evaluates the effect of scaffold-induced compression on fibrocartilage development on a tendon graft, focusing on matrix remodeling and the development of fibrocartilage-related markers. Another objective is to compare effects of scaffold-induced dynamic and static compression on a tendon graft. Another objective is to compare effects of scaffold-induced dynamic and static compression on a tendon graft.

This study focuses on the design and evaluation of a mechano-active scaffold system based on a composite of poly-α-hydroxyester nanofiber mesh and sintered microspheres. Specifically, the effects of scaffold-induced compression on tendon matrix remodeling and the development of fibrocartilage-related markers are evaluated over a two-week period. Scaffold contraction resulted in over 15% compression of the patellar tendon graft and up-regulated the expression of fibrocartilage-related markers such as type II collagen, aggrecan and transforming growth factor-53. In addition, proteoglycan content was significantly higher in the compressed tendon group after one day of loading. Further, the effects of scaffold-induced dynamic and static compression on a tendon graft were compared. It was found that static compression produced greater compression in the fibers, greater retention of matrix proteoglycan, higher cell number count, and greater expression of fibrocartilage-related markers.

This is the first reported study describing scaffold-mediated mechanical loading, and the findings of this study demonstrate the potential of the mechano-active scaffold to promote the formation of an anatomic fibrocartilage transition on tendon-based ACL reconstruction grafts, which is critical for achieving biological fixation and extending graft functionality. It is envisioned that the mechano-active scaffold complex can be used clinically to apply both biochemical and mechanical stimuli to induce metaplasia of the tendinous matrix, ultimately facilitating the formation of an anatomic fibrocartilage interface on these grafts. This approach offers significant promise as the functional transition between soft tissue and bone would be re-established, with the potential to ensure long-term graft stability and improve clinical outcome through biological fixation.

### Materials and Methods

### Tendon Graft Isolation

Patellar tendon grafts were isolated from neonatal bovine tibiofemoral joints (1-7 days old) obtained from a local abattoir (Green Village Packing, Green Village, N.J.). Briefly, the joints were first cleaned in an antimicrobial bath. Under antiseptic conditions, midline longitudinal incisions were made through the subcutaneous fascia to expose the patellar tendon. The paratenon was removed, and the patellar tendon dissected from the underlying fat pad. Sharp incisions were made through the patellar tendon at the patellar and tibial insertions, and the insertions were completely removed from the graft.

### Nanofiber Mesh Fabrication and Characterization

For the nanofiber mesh fabrication, polymer nanofiber meshes (both aligned and unaligned) were fabricated by electrospinning.

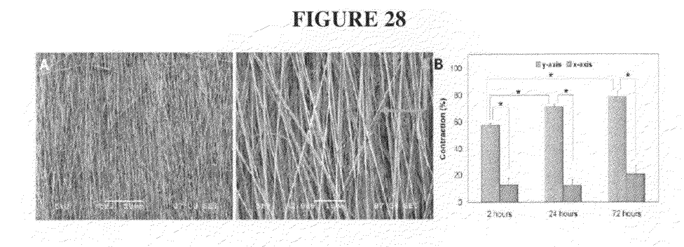

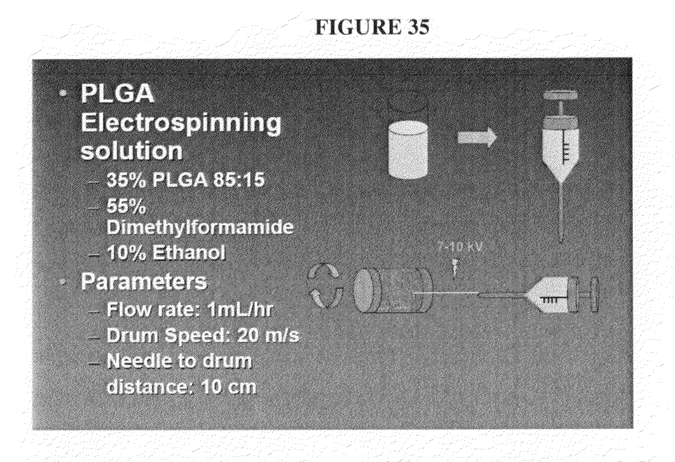

Aligned nanofiber meshes (FIGS. 28A and 28B) were fabricated by electrospinning (Doshi, 1995). A viscous polymer solution consisting of 35% poly(DL-lactic-co-glycolic acid) 85:15 (PLGA, I.V.=0.70 dL/g, Lakeshore Biomaterials, Birmingham, Ala.), 55% N,N-dimethylformamide (Sigma, St. Louis, Mo.), and 10% ethanol (Commercial Alcohol, Inc., Toronto, Ontario) was loaded into a syringe fitted with an 18-gauge needle (Becton Dickinson, Franklin Lakes, N.J.). Aligned fibers (Yang, 2005) were obtained using an aluminum drum with an outer diameter of 10.2 cm rotating with a surface velocity of 20 m/s. A constant flow rate of 1 mL/hr was maintained using a syringe pump (Harvard Apparatus, Holliston, Mass.), and an electrical potential was applied between the needle and the grounded substrate (distance=10 cm) using a high voltage DC power supply (Spellman, Hauppauge, N.Y., 8-10 kV). (FIG. 35) Fiber morphology, diameter and alignment of the as-fabricated mesh samples were analyzed using scanning electron microscopy (SEM). Briefly, the samples were sputter-coated with gold (LVC-76, Plasma Sciences, Lorton, Va.) and subsequently imaged (JSM 5600LV, JEOL, Tokyo, Japan) at an accelerating voltage of 5 kV.

The aligned mesh was cut into 15.5 cm×1.5 cm strips.

### Graft Collar Scaffold Fabrication

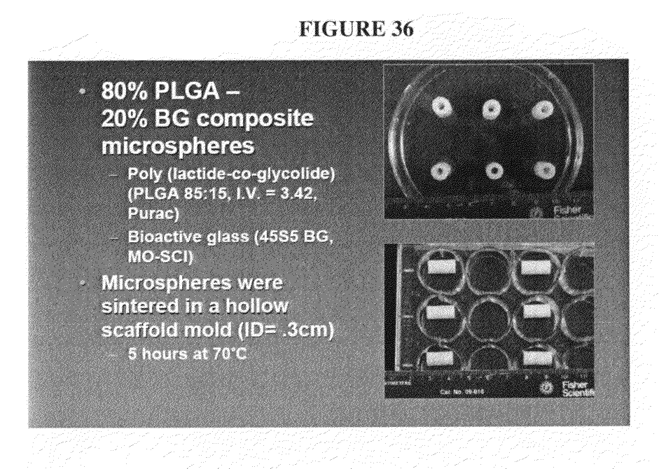

A tendon graft collar based on a sintered microsphere scaffold was fabricated following published methods (Lu, 2003; Spalazzi, 2006) Specifically, the scaffold is composed of composite microspheres consisting of PLGA (85:15, I.V.=3.42 dl/g, Purac, Lincolnshire, Ill.) and 45S5 bioactive glass (BG, 20 μm, MO-SCI Corporation, Rolla, Md.). The microspheres were formed following the methods of Lu et al. (Lu, 2003) where the polymer was first dissolved in dichloromethane (Acros Organics, Morris Plains, N.J.) and then BG particles were added (20 wt %). After vortexing, the suspension was poured into a 1% solution of polyvinyl alcohol (Sigma, St. Louis, Mo.) to form the microspheres. The microspheres were subsequently sintered at 70° C. for 5 hours in a custom mold to form cylindrical scaffolds with an outer diameter of 0.7 cm and an inner diameter of 0.3 cm. (FIG. 36)

### Characterization of Nanofiber Mesh Contraction

Mesh contraction was evaluated using digital image analysis. Briefly, the nanofiber meshes were cut into 10 mm×10 mm squares and immersed in Dulbecco's Modification of Eagle's Medium (DMEM, Mediatech, Inc., Herndon, Va.) supplemented with 10% fetal bovine serum (FBS, Atlanta Biologicals, Norcross, Ga.) and incubated at 37° C. and 5% CO₂. The meshes were imaged using stereomicroscopy at 0, 2, 24, and 72 hours. Mesh dimensions (n=5) were measured by image analysis (ImageJ 1.34 s, NIH, Bethesda, Md.), and contraction was calculated based on percent change in length both in the x-axis and along the direction of fiber alignment (y-axis).

Compression of Graft Collar Scaffold with Nanofiber Mesh

In addition to mesh contraction, the nanofiber mesh-mediated compression of the microsphere-based graft collar was also evaluated in vitro. Briefly, strips of nanofiber mesh (15.5 cm×1.5 cm) were wrapped around the graft collar scaffold, with the fibers aligned perpendicular to the scaffold long axis. The mesh+scaffold was then incubated in PBS at 37° C. and 5% CO₂, and changes in scaffold diameter (n=6) due to mesh contraction were monitored over 24 hours using image analysis (ImageJ).

Compression of Tendon with Nanofiber Mesh

The potential of utilizing nanofiber mesh contraction to directly apply compression to the tendon graft was evaluated over time. Briefly, the aligned electrospun meshes were cut into 10 cm×2 cm strips, with fiber alignment oriented along the long axis of the mesh. The patellar tendon graft was bisected along its long axis, and one half of the tendon was wrapped with the nanofiber mesh while the other half served as the unloaded control (FIG. 30A). The samples were cultured in DMEM supplemented with 1% non-essential amino acids, 1% antibiotics, and 0.1% antifungal (all from mediatech) and 10% FBS (Atlanta Biologicals). At days 5 and 14, the effects of compression on tissue morphology and cellularity were characterized by histology (Spalazzi, 2006) The samples were rinsed with phosphate buffered saline (PBS, Sigma), fixed with 10% neutral buffered formalin (Fisher Scientific and Sigma) and embedded in paraffin (Fisher Scientific, Pittsburgh, Pa.). The samples were then cut into 7-μm thick sections and stained with hematoxylin and eosin (H&E).

Compression of Tendon Graft with the Graft Collar Scaffold and Nanofiber Mesh

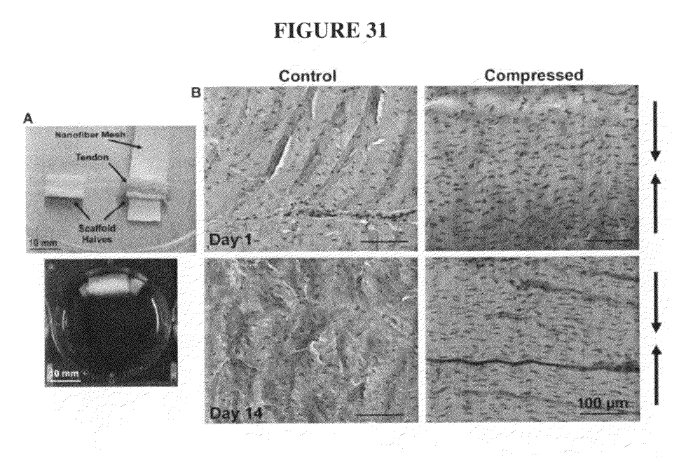

The potential of the graft collar scaffold and nanofiber mesh complex to apply static compression to the patellar tendon graft was also evaluated in vitro. Specifically, the patellar tendon graft was dissected into 2 cm×0.3 cm segments and the cylindrical scaffold was halved along its long axis. Each tendon segment was inserted between the two scaffold halves (FIG. 31A, top). For the experimental group, the tendon+graft collar was wrapped with the aligned nanofiber mesh (15.5 cm×1.5 cm), while the control scaffolds were wrapped with pre-contracted electrospun mesh (n=2). In addition, to ensure static compression of the tendon graft, the experimental group was wrapped with new mesh strips on every other day during the two week culturing period. The scaffold+tendon graft complex (FIG. 31A, bottom) was cultured in fully supplemented media at 37° C. and 5% CO₂.

### Effects of Compression on Tendon Graft Cellularity and Matrix Content

The effects of static compression on tendon matrix organization (n=2) were analyzed at 1 and 14 days via histology (H&E). Collagen distribution and organization was visualized using Picrosirius red stain and examined under polarized light microscopy.

In addition, since most of the mesh compression occurs within the first 24 hours, total cell number (n=5) and proteoglycan content in the tendon graft were evaluated at day 1.

Cellularity and proteoglycan content were measured from days 0, 1, 3, 7 and 14. For the biochemical assays (Jiang, 2007; Jiang, 2005; Spalazzi, 2006), both the wet and dry weights of the tendon samples were determined at day 0 and day 1, and the tissue was subsequently digested for 16 hours in 2% papain (Sigma) buffer at 60° C. Total DNA content of the digest was determined with the PicoGreen dsDNA assay (Molecular Probes), following the manufacturer's suggested protocol. Sample fluorescence was measured using a microplate reader (Tecan, Research Triangle Park, N.C.), with excitation and emission wavelengths set at 485 and 535 nm, respectively. The total number of cells in the sample was calculated using the conversion factor of 8 pg DNA/cell (Lu, 2005).

Sulfated glycosaminoglycan (GAG) content was quantified using a colorimetric 1,9-dimethylmethylene blue (DMMB) assay. Tissue digest from the cell quantitation assay was combined with DMMB dye, and the concentration of GAG-DMMB complexes was determined using a plate reader at 540 and 595 nm and correlated to a standard prepared with chondroitin-6-sulfate.

### Cell Phenotype

Gene expression for fibrocartilage markers (n=2) such as collagen I, II, aggrecan, and Transforming Growth Factor-Beta 3 (TGF-β3) was determined at day 1 using reverse-transcription polymerase chain reaction (RT-PCR). Briefly, after removing the graft collar and nanofiber mesh, total RNA of the tendon graft was obtained using the Trizol extraction method (Invitrogen, Carlsbad, Calif.). The isolated RNA was reverse-transcribed into cDNA using the SuperScript III First-Strand Synthesis System (Invitrogen, Carlsbad, Calif.) and the cDNA product was amplified using recombinant Platinum Taq DNA polymerase (Invitrogen). GAPDH was used as the housekeeping gene, and expression band intensities were measured (Imagej) and normalized against GAPDH.

### Statistical Analysis

Results are presented in the form of mean±standard deviation, with n equal to the number of samples analyzed. Two-way analysis of variance (ANOVA) was first performed to assess if differences exist among the means. Fisher's LSD post-hoc test was subsequently performed for cellularity and GAG quantification for all pair-wise comparisons to determine the significance of effects between the control, dynamic and static compression groups as well as a function of culturing time. Statistical significance was attained at p<0.05. For gene expression, a one-way ANOVA and Fisher's LSD post-hoc test were performed to determine the effect of the compression types on type II collagen, aggrecan and TFG-B3. All statistical analyses were performed using the JMP statistical software package (SAS Institute, Cavy, N.C.).

### Comparison of Effects of Scaffold-Induced Dynamic and Static Compression on a Tendon Graft

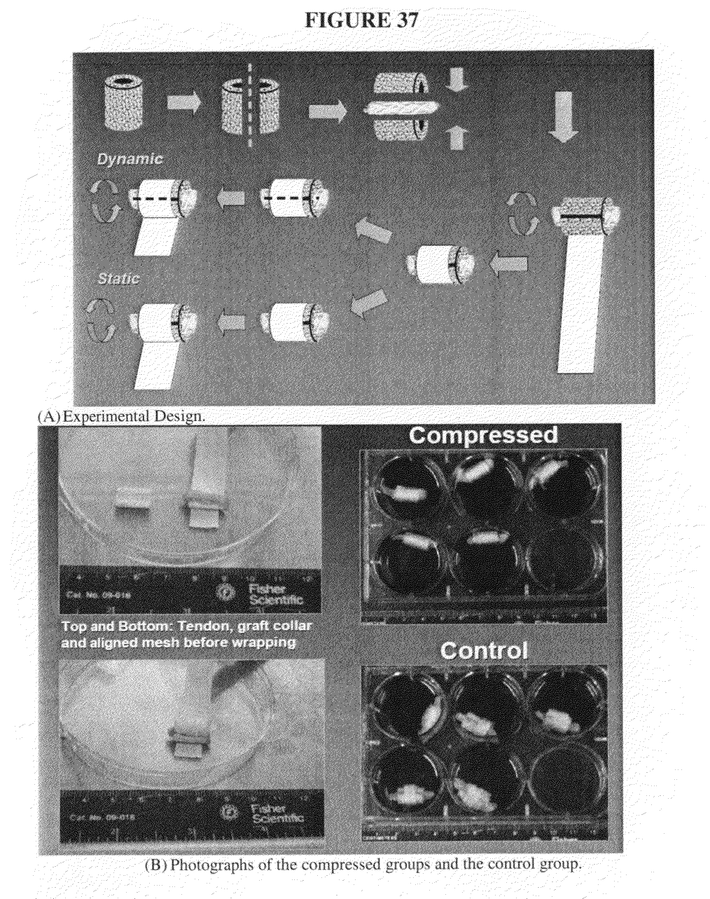

Three groups of scaffold and graft apparatus were used: the dynamic compression group, the static compression group and the control group. (FIG. 37A-37B)

For the static compression group, a tendon segment was placed in the hollow of a bisected scaffold collar and the collar-tendon structure was wrapped approximately 5 times with the aligned electrospun mesh. The mesh-collar-tendon was incubated in media. The aligned electrospun mesh was allowed to contract over the first 24 hours. After the initial contraction, the collar complex remained in static compression for a further 24 hours before being wrapped in another aligned mesh strip, with this pattern continuing for 14 days.

For the dynamic group, the above procedure was followed until the static compression phase. After the initial 24 hour mesh contraction, the mesh was cut along the seams of the microsphere scaffold, releasing the compression. The collar complex was allowed to relax for 24 hours before being wrapped with another aligned mesh strip with this pattern continuing for 14 days.

For the zero-compression control, a collar-tendon complex was wrapped in pre-contracted unaligned electrospun mesh.

The above groups were incubated in Dulbecco's Modification of Eagle's Medium (DMEM) supplemented with 10% Fetal Bovine Serum, 1% antibiotics, 1% non-essential amino acids and 0.1% antifungal at 37° C. and 5% CO₂.

### Results

### Nanofiber Characterization and Mesh Contraction

The nanofiber mesh exhibited a high degree of alignment with an average fiber diameter of 0.9±0.4 μm (FIG. 28A). Anisotropic mesh contractile behavior was observed in the mesh, with significantly higher contraction found in the direction of nanofiber alignment. Specifically, the mesh contracted over 57% along the aligned fiber direction (y-axis) by 2 hours, with less than 13% reduction in the x-axis (FIG. 28B). Mesh contraction continued over time, exhibiting over 70% contraction in the y-axis and 20% in the x-axis by 24 hours and stabilizing thereafter, with no significant differences found between the 24- and 72-hour groups.

Compression of Graft Collar Scaffold with Nanofiber Mesh

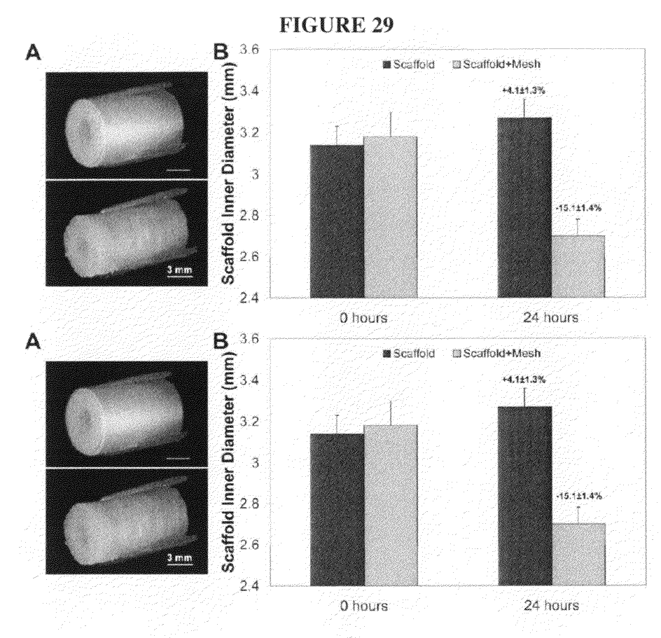

After the nanofiber mesh was wrapped around the graft collar scaffold, mesh contraction resulted in a significant decrease in scaffold inner diameter, averaging 15% strain within 24 hours (FIG. 29). In contrast, the control scaffold without mesh cultured under similar conditions expanded and measured an increase in inner diameter (4%), although the difference was not statistically significant (p<0.05).

Compression of Tendon with Nanofiber Mesh

When the nanofiber mesh was used to compress the tendon graft, mesh contraction resulted in an approximately 30% decrease in graft diameter by 24 hours (FIG. 30A). After five days of explant culture, the compressed tendon exhibits less of the crimp structure evident in the control group, and remodeled into a dense matrix with high cellularity (FIG. 30B). However, by day 14, the crimp pattern was restored in the compressed group, with ultrastructure and cellularity indistinguishable from the unloaded control group.

Compression of Tendon with the Graft Collar Scaffold and Nanofiber Mesh

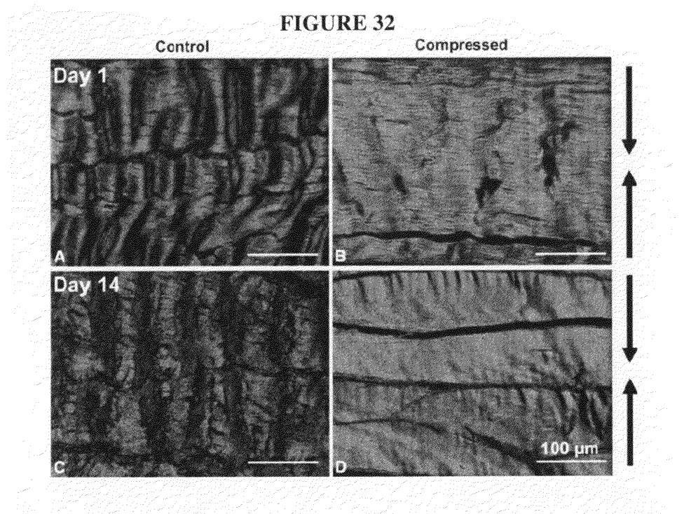

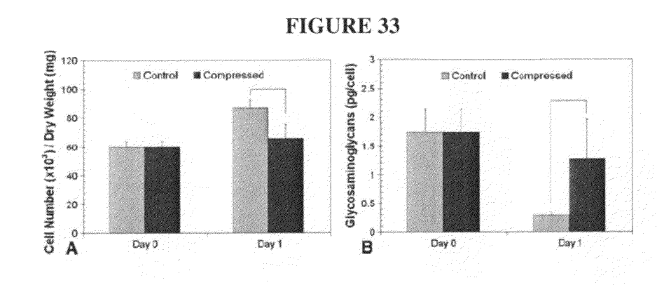

In order to apply a physiological level of loading (10-15%), the tendon graft was compressed by a complex of the graft collar scaffold and nanofiber mesh. It was observed that at 24 hours post-compression (FIG. 31B, top), the tendon graft matrix organization was distinct from that of the unloaded control, with increased matrix density and less of the characteristic crimp of the tendon. After 14 days of compression by the scaffold+mesh complex, it was found that the matrix remodeling visible 24 hours following the onset of loading was maintained over time (FIG. 31B, bottom). In contrast, the control tendon retained its characteristic crimp, with evident disruption of the matrix ultrastructure. Further, compression distinctly changed matrix collagen organization. The color of collagen fibers stained with Pricrosirius red and viewed under polarized light is reported to correlate with fiber diameter, (Hiss, 1988; Junqueira, 1982; Rich 2005) progressing from green, yellow, orange to red with increasing fiber diameter. While no change in fiber diameter was observed in the unloaded control group (FIG. 32A), the collagen fiber diameter of the group compressed with the mesh+collar scaffold became smaller (in green) after 24 hours of loading (FIG. 32B). Moreover, disruption of the tendon collagen matrix was evident in the control group by day 14 (FIG. 32C). In general, collagen fibers remained perpendicular to the direction of loading after 24 hours, and this effect was maintained over 14 days with the mesh+collar scaffold complex (FIG. 32D). In addition to changes in tendon matrix organization, total cell number in the tendons remained relatively constant in the compressed group, with a significantly higher number of cells found in the control tendons by day 1 (FIG. 33A). Interestingly, matrix glycosaminoglycan (GAG) content was found to be significantly higher in compressed tendon group after one day of culture (FIG. 33B).

### Effects of Compression on the Expression of Fibrocartilage-Related Markers

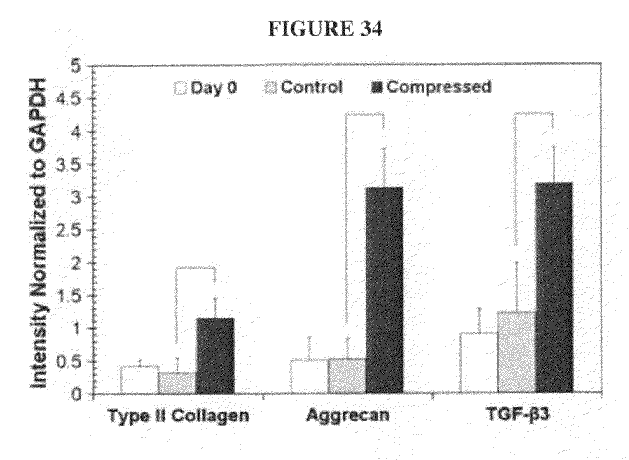

The expression of fibrocartilage markers such as types I and II collagen, aggrecan and TGF-β3 were evaluated after compression with the graft collar scaffold and nanofiber mesh. The expression of collagen II, aggrecan and TFG-B3 were evaluated at Days 1, 3 and 7. As shown in FIG. 34, after 24 hours of compression, gene expression of type II collagen, aggrecan and TGF-β3 were all up-regulated in the loaded group when compared to non-compressed tendons (FIG. 34), with significant differences found in aggrecan and TGF-β3 expression. Specifically, in the static group, on average all genes experienced upregulation for all timepoints compared to control, with aggrecan significantly upregulated on days 1 and 3, and collagen II and TGF-β3 significantly upregulated on day 3. In the dynamic group, all genes were upregulated compared to control on days 1 and 3, with collagen II significantly upregulated on day 3.

### Comparison of Effects of Scaffold-Induced Dynamic and Static Compression on a Tendon Graft

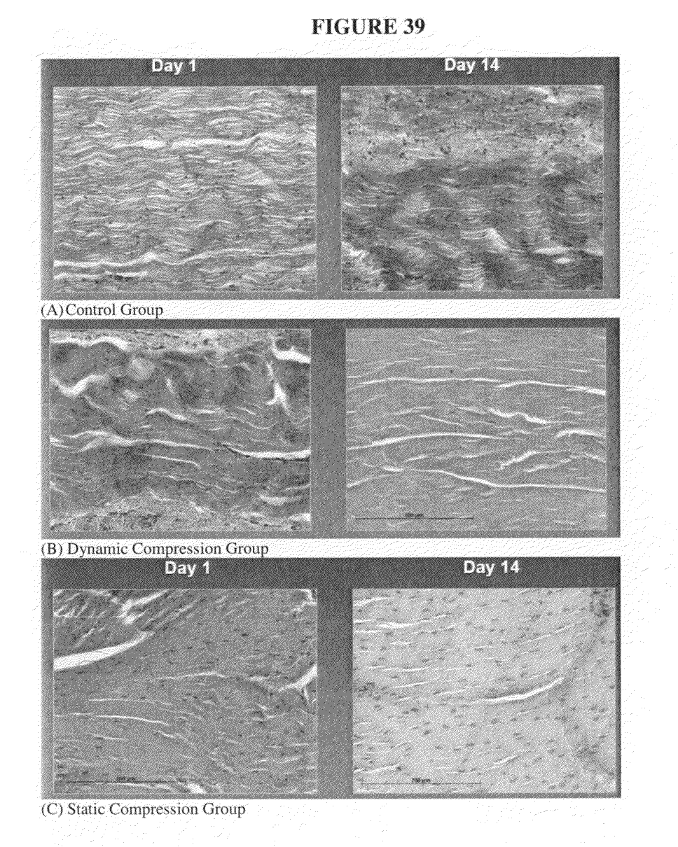

The morphology of the control group is maintained from day 1 to day 14. Crimp in the tissue was maintained. In both the dynamic and the static compression group, fiber morphology was compressed after day 1 and continues to compress to day 14. That is, in the static compression group, for both time points, the fiber organization is distinctly different from the unloaded control at both time points, featuring a more tightly packed tissue morphology and loss of the characteristic crimp. (FIG. 39)

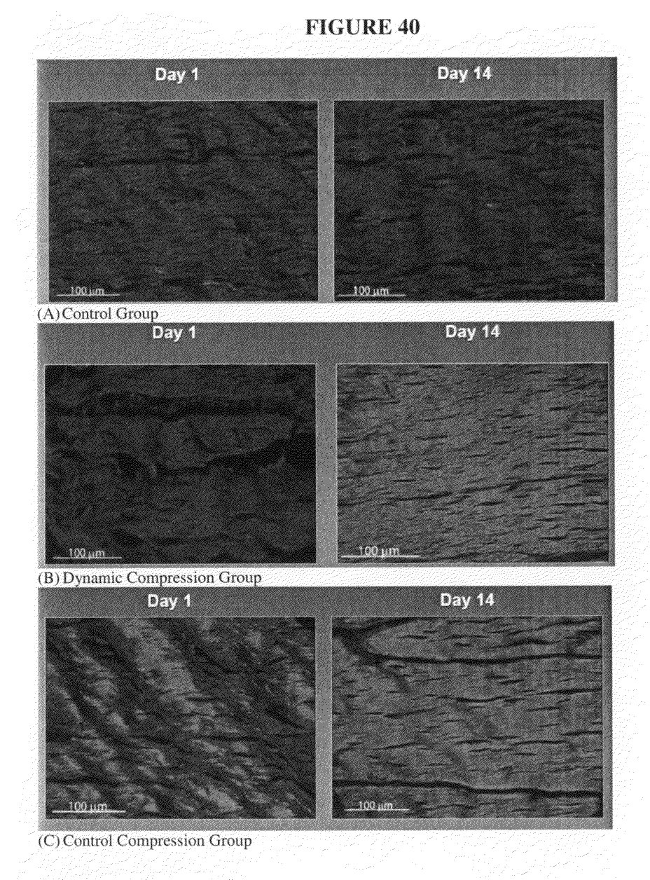

Graft Collagen Fiber Diameter did not change in the control group. For the static compression group, a decreased collagen fiber diameter and greater collagen alignment was shown. There was little fiber diameter change at day 1 and notable fiber diameter decrease by day 14. For the dynamic compression group, differences from the unloaded control were similar to the static group and also included tightly packed tissue morphology, loss of crimp and increased alignment and decreased collagen fiber, however, these differences were only observed at day 14. After day 1 there was no change. (FIG. 40)

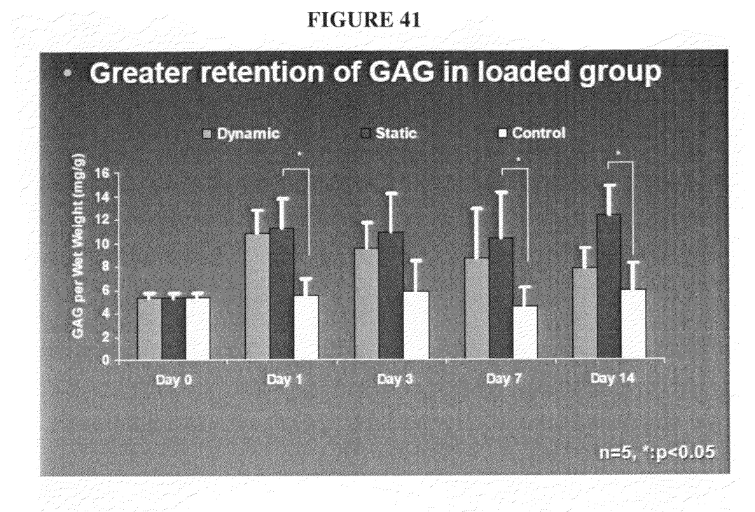

Further, there was greater retention of matrix proteoglycan content in the compressed group, especially in the static compression group through day 14. (FIG. 41) Glycoaminoglycan (GAG) content was quantified through the DMMB dye assay and measured from day 0 to day 14. On average, the static compression group had the highest GAG content with significant differences noted. It was also observed that the GAG level was maintained in the static compression group over the 14 days while the dynamic group experienced a downward trend.

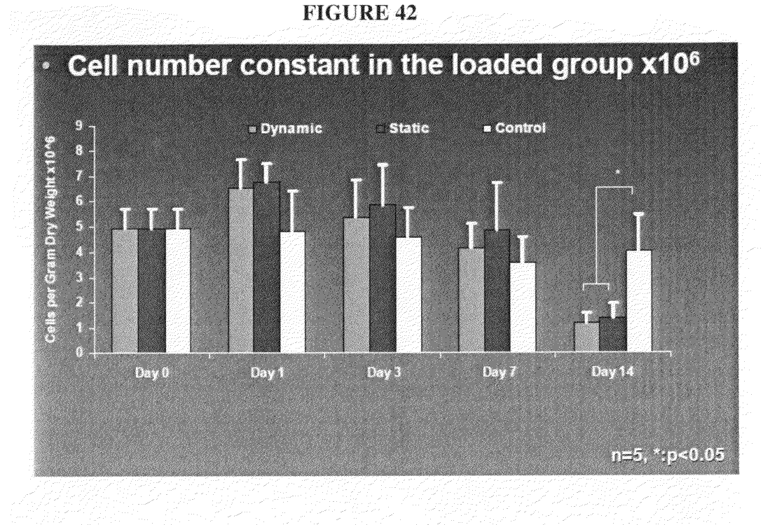

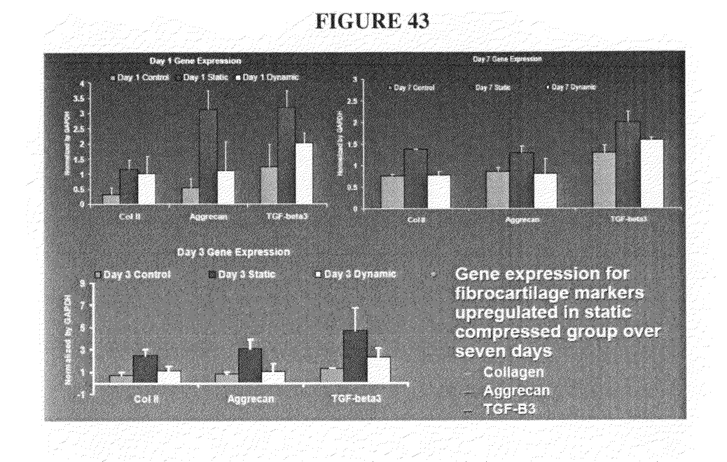

In addition, the cell number is greater in the static compression group than in the dynamic compression group. (FIG. 42) Additionally, gene expression for fibrocartilage markers up-regulated in static compressed group over seven days. (FIG. 43)

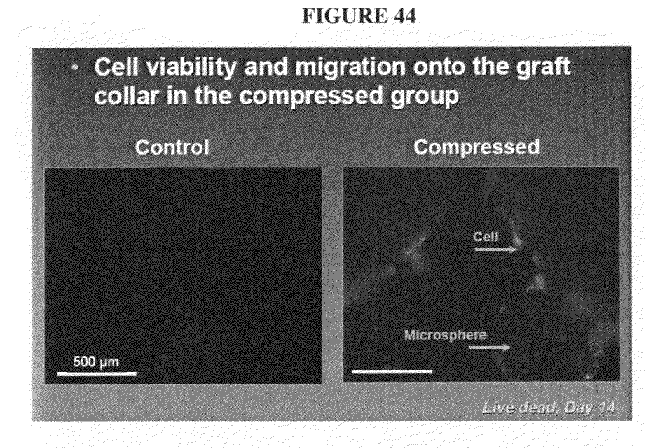

Finally, cell viability and migration onto the graft collar was observed in the compressed groups but not in the control group. (FIG. 44)

### Effects of Compression on Tendon Matrix-Preliminary In Vivo Study

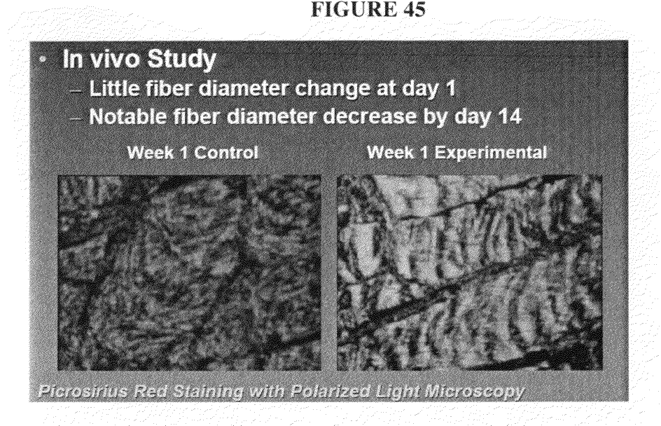

After 1 week of implantation, the re-organization of the tendon due to mesh contraction was maintained without re-wrapping. There was little fiber diameter change at day 1. However, there was notable fiber diameter decreases by day 14. (FIG. 45)

### Discussion

The long term goal is to achieve biological fixation by engineering a functional and anatomical fibrocartilage interface on biological and synthetic soft tissue grafts used in orthopaedic repair (Lu, 2006). To this end, the current study focuses on the design and evaluation of a novel graft collar scaffold system capable of applying mechanical loading and inducing fibrocartilage formation on tendon grafts. Specifically, scaffold-mediated compression of a patellar tendon graft was evaluated over time, focusing on the effects of loading on tendon matrix organization and cell response. In addition, effects of scaffold-induced dynamic and static compression on a tendon graft were compared. It was found that the complex of the nanofiber mesh and graft collar was able to apply a physiological range of compressive loading to tendon grafts. Moreover, scaffold-mediated compression promoted matrix remodeling, maintained graft glycosaminoglycan content and, interestingly, induced gene expression for fibrocartilage markers, including type II collagen, aggrecan, and TGF-β3.

Further, static compression was found to be more effective in producing changes in graft collagen fiber diameter, increasing matrix proteoglycan content, cell number and gene expression for fibrocartilage markers. These promising results demonstrate that compressive loading can be incorporated into scaffold design and used to promote fibrocartilage formation on tendon grafts.

Two scaffold-based loading systems were described in this study. The first design involved using a nanofiber mesh to directly load the tendon graft. The pre-designed alignment of the nanofiber mesh results in anisotropic mesh contractile behavior, effectively translating contractile force into compression, which has been utilized in this study to apply compressive loading to the tendon grafts. Histological analysis of the grafts revealed that the scaffold-mediated compression induced extensive remodeling of the tendon ultrastructure, with the compressed graft exhibiting a denser matrix with increased local cell density. This matrix modulation effect, however, diminished over time, with the control and loaded groups nearly indistinguishable by day 14. As mesh contraction stabilizes after 24 hours, it is likely that the tendon graft is no longer experiencing mechanical stimulation in long term cultures. These observations suggest that it is necessary to incorporate extended mechanical stimulation into scaffold design.

The short-term effect of mesh-induced compressive loading on graft matrix organization and the high magnitude of compression (approximately 30%) initiated the development of the second mechano-active scaffold system. Specifically, the nanofiber mesh was combined with a degradable microsphere-based graft collar system in order to achieve a physiological range of loading (15%). Moreover, to maintain static compression, the tendon-scaffold complex was wrapped with new nanofiber mesh every other day. It was observed that under static compression, the remodeled tendon matrix with cells embedded in a dense matrix was maintained over time, with marked differences observed between control and the loaded groups. These observations demonstrate the potential of this scaffold system to provide continuous mechanical stimulation and promote sustained tissue remodeling. Proteoglycan content of the tendon matrix was also significantly higher in the compressed group compared to the control at day 1, further indicating that the scaffold-induced compression influences matrix maintenance and remodeling.

Scaffold-mediated compression also resulted in the up-regulation of fibrocartilage markers including type II collagen, aggrecan, and Transforming Growth Factor-β3 (TGF-β3). It is well known that fibrocartilage in tendons is largely comprised of types I and II collagen, as well as proteoglycans (Benjamin, 1998; Evanko, 1993; Koob, 1992; Milz, 1998). Moreover, compressive loading of fibrocartilaginous regions of tendons has been reported to increase the synthesis of Transforming Growth Factor-β1 (TGF-β1) (Robbins, 1997) and large proteoglycans, as well as enhancing aggrecan gene expression (Evanko, 1993; Koob, 1992). Compression of the non-fibrocartilaginous regions of the deep flexor tendon has also been reported to promote proteoglycan synthesis (Evanko, 1993). The findings of this study are in agreement with these published studies on the effects of compressive loading, and demonstrate the feasibility of implementing a degradable scaffold system for fibrocartilage interface formation on tendon grafts. In addition to applying continuous compressive loading to the graft within a physiological range, it is anticipated that this novel scaffold system also can be used to deliver cells and growth factors. These design optimizations will be critical for allograft re-cellularization and exercising biochemical stimulation to direct cellular differentiation as well as transformation of the tendon matrix into fibrocartilage.

It was found that scaffold mediated static loading promoted matrix remodeling, increased graft glycoaminoglycan content and induced gene expression of fibrocartilage related markers such as type II collagen, aggrecan and TGF-β3 compared to the control. Scaffold mediated dynamic loading was also found to promote matrix remodeling and increased graft GAG content, though to a lesser extent than the static group. These results indicate both that compressive loading can be incorporated into scaffold designs and furthermore that compressive loading may be necessary to develop the fibrocartilage interface.

Two types of scaffold-based loading systems were described in this study. The first involved using an aligned nanofiber mesh to apply static loading to a microsphere graft collar. The nanofibers contract along their long axis, translating contractile force into compression of the microsphere collar. Histological analysis revealed that the scaffold mediated compression induced remodeling of the tendon ultrastructure, including a denser matrix, loss of characteristic crimp and smaller collagen fiber diameter through 14 days.

The second type of scaffold incorporates full graft relaxation in between cyclic loadings, in order to mimic physiologic loading patterns. Specifically, the mechanical loading of a tendon when the animal is moving and relaxation when the animal is asleep. Histological analysis revealed that the scaffold mediated compression with relaxation induced remodeling of the tendon ultrastructure to a lesser extent than the static compression over 14 days, suggesting that the total compressive loading over 14 days was less than in the static group.

Both the static and dynamic loading groups show higher proteoglycan content compared to control over 14 days. However, despite having similar levels at day 1, proteoglycan content in the dynamic group experienced a steady decreasing trend over 14 days while the proteoglycan content in the static group was maintained. Combined with the histological data, this suggests that the mechanical loading was maintained over 14 days and furthermore that such maintenance of loading is necessary for fibrocartilage formation. Furthermore, gene expression data shows upregulated expression of fibrocartilage markers such as type II collagen, aggrecan and TGF-β3 over 7 days. Fibrocartilage in tendons is mostly comprised of types I and II collagen as well as proteoglycans. Studies have reported that compressive loading of tendons results in higher aggercan production, TGF-β synthesis and large proteoglycans. Studies involving application of compressive loading to non-fibrocartilaginous regions of the deep flexor tendon have resulted in increased proteoglycan synthesis. Regarding static vs. dynamic loading, the findings from this study is not in agreement with published studies (Vogel et al.) which conclude that cyclic compression and relaxation results in higher proteoglycan content. However, Vogel et al. used a much higher rate of compression-relaxation compared to this compression-relaxation cycle speed. In both compression groups, tendon cell number is maintained until day 14 at which point there is significant decreased compared to control. It is believed that multiple layers of scaffold and mesh, both of which are hydrophobic, may be impeding nutrients from reaching the tendon grafts, resulting in cell death after between 7 and 14 days of culture.

It is anticipated that this mechano-active scaffold can not only apply both dynamic and static compressive loading, but also could be used for the purpose of delivering growth factor or cells to the interface site.

Contraction of PLGA meshes has been previously reported in the literature (Zong, 2003), although the phenomenon has been discredited as a shortcoming rather than promoted as an advantageous attribute of the system. Currently, the mechanism underlying mesh contraction is not known. Zong et al. (Zong, 2003) have observed that electrospun nanofiber mesh comprised of crystalline polyesters contract significantly less than amorphous polyester co-polymers such as PLGA 75:25. It was proposed that when nanofiber meshes comprised of crystalline polymers are incubated at 37° C., the polymer glass transition temperature is approached and crystallization rapidly occurs, resulting in a lamellar structure that constrains the relaxation of the polymer chains and in turn prevents contraction (Zong, 2003). The polyester co-polymer utilized in this study has a high D,L-lactide content (85%) and is non-crystalline, thus the above mechanism may explain the high degree of contraction observed. Although not the focus of the current study, fiber alignment-related scaffold anisotropy may be controlled to modulate mesh contraction, and consequently, the magnitude and direction of compressive loading on the graft may be controlled by customizing the degree of fiber alignment. Future studies will focus on elucidating the mechanism of mesh contraction as well as exploring methods to control this process for mechanical stimulation.

This is the first study to incorporate mechanical loading into scaffold design and to demonstrate the potential of using this mechano-active scaffold system to induce fibrocartilage formation on soft tissue grafts. The mesh-collar system is intended to be applied clinically as a degradable graft collar, and may used to initiate and direct regeneration of an anatomical fibrocartilage interface at the insertion of tendon-based ACL reconstruction grafts. In addition to providing a three-dimensional environment for matrix development and growth factors for guided cell differentiation, the innovative scaffold system described here can also apply physiologic mechanical stimulation crucial for directing cellular function and tissue remodeling. For utilization with viable autografts, it is envisioned that the graft would be inserted through the collars immediately prior to implantation, and compression of the graft and subsequent fibrocartilage formation would occur in vivo. Allografts, which do not contain viable cells necessary for remodeling the tendon matrix, would need to be repopulated with fibroblasts or stem cells delivered either from the scaffold in vitro prior to graft implantation. It has been reported that mesenchymal stem cell (MSC)-seeded type I collagen sponges inserted into excised sheep patellar tendons and loaded using an ex vivo wrap-around system results in an up-regulation of chondrogenic markers such as Sox9 and Fos (Li, 2006). A similar response by a cell-populated tendon allograft is anticipated following scaffold-mediated compressive loading. Moreover, the mesh-scaffold system is based on degradable poly-α-hydroxyester polymers, thus it is expected that the mechano-active scaffold will be replaced by newly formed tissue after a functional fibrocartilage interface has been formed on the graft.

### Experiment 6.2

### Perpendicular and Parallel Wrapping of Tendon by PLGA Electropun Mesh

The objective of this experiment was to determine the effect of wrapping tendon with a PLGA electrospun mesh wherein the fibers of the mesh were either perpendicular or parallel to the longitudinal axis of the tendon.

Three groups (3 tendons per group) were examined: (1) A control group with no mesh wrapping; (2) a group wrapped with mesh, wherein the fibers of the mesh were perpendicular to the longitudinal axis of the tendon; and (3) a group wrapped with mesh, wherein the fibers of the mesh were parallel to the longitudinal axis of the tendon. The meshes were allowed to contract for 48 hours. See FIGS. 46A and 46B.

### Results

The control group exhibited a 13.3±6.4 percentage change in tendon diameter and a −6.2±5.2 percentage change in tendon length. The perpendicular fiber group exhibited a −40.0±63.6 percentage change in tendon diameter and a 12.9±2.2 percentage change in tendon length. The parallel fiber group exhibited a 5.6±6.7 percentage change in tendon diameter and a −16.3±5.6 percentage change in tendon length.

### Discussion

This experiment indicates that wrapping a tendon with a PLGA electrospun mesh having fibers perpendicular to the longitudinal axis of the tendon results in decreased tendon diameter and increased tendon length (due to compression of the center of the tendon). Wrapping a tendon with a PLGA electrospun mesh having fibers parallel to the longitudinal axis of the tendon results in decreased tendon length and no significant change in tendon diameter compared to the control.

### Experiment 7

### The Triphasic Scaffold

Clinically, the hamstring tendon graft is mechanically fixed extra-articularly by looping the graft around a transfemoral pin in the femoral bone tunnel, while a screw with a washer or a staple is used to fix the graft to the tibia. Interference screws have been used in the bone tunnel, but with limited success due to graft laceration and poor fixation strength. With mechanical fixation, the fibrocartilage interface is not regenerated after ACL reconstruction. A non-physiologic, fibrovascular scar tissue is instead formed within the bone tunnel as part of the healing process. The presence of this partially mineralized layer within the tunnel renders the graft-bone fixation site the weakest point mechanically (Kurosaka, 1987). This problem is exacerbated by the active lifestyle of ACL injury patients (15-35 years old), which necessitates higher fixation strength and expedited healing. Thus, graft-to-bone fixation remains a significant clinical problem.

The subject approach to addressing the challenge of biological fixation is original and represents a significant departure from the conventional focus on tendon-to-bone healing within the bone tunnel. It is emphasized here that the native anatomical fibrocartilage interface is orthogonal to the subchondral bone and continuous with surrounding articular cartilage. In addition, the neo-fibrocartilage formed within the bone tunnel represents the mechanical weak link for tendon-to-bone integration. Biological fixation therefore requires that the anatomical fibrocartilage insertion is regenerated between graft and bone, accompanied by the complete mineralization of the tendon within the bone tunnel.

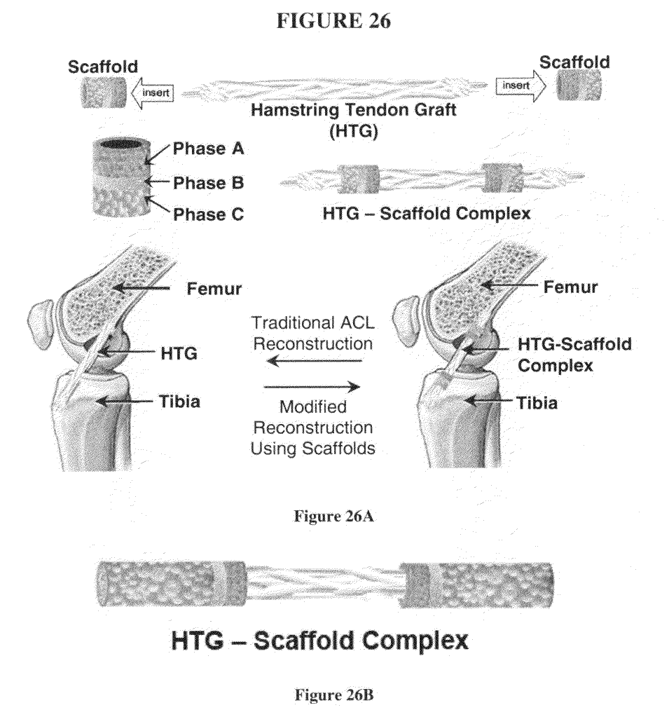

It is envisioned that the triphasic scaffold may be used clinically as either as a graft collar or an interference screw during ACL reconstruction surgery. The ultimate goal is to facilitate the formation of the anatomic fibrocartilage interface directly on the soft tissue graft. As a graft collar, the scaffold can be fabricated as a hollow cylinder through which the ACL graft can be inserted. As shown in FIG. 26A-B, the collar can be sutured or secured to the ends of the tendon graft. Fixation is achieved by inserting the collar-graft complex into the bone tunnel, with Phase C positioned inside the bone tunnel, Phase B flush with articular cartilage, and only Phase A directly exposed to the joint cavity. It is anticipated that the designed heterogeneity and optimized interaction between MSC-derived cells will induce the formation of a fibrocartilage interface directly onto the graft. Graft integration within the bone tunnel will be facilitated by Phase C, the osteointegrative polymer-ceramic composite, and with the eventual addition of growth factors (e.g., bone morphogenetic proteins), which will induce osteointegration and mineralization of the tendon graft within the bone tunnel.

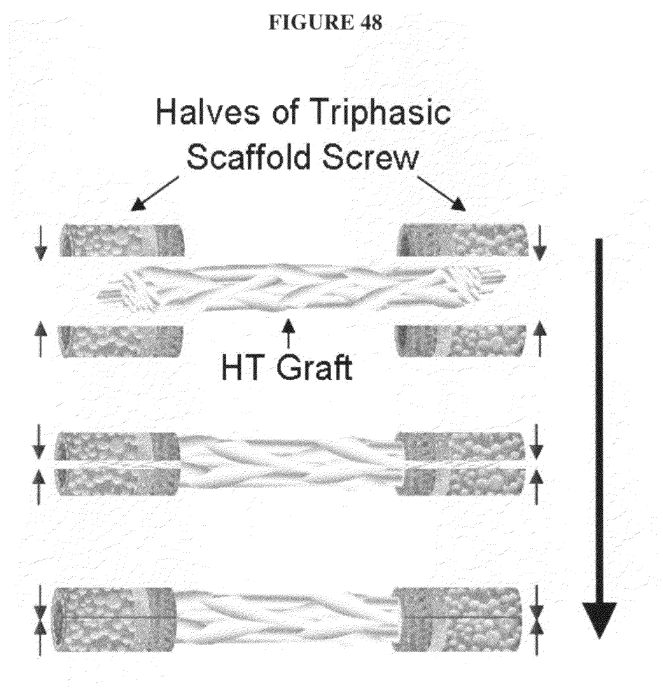

For use as an interference screw, the triphasic scaffold can be fabricated as matching portions of the hollow cylinder, with each portion containing the three scaffold phases. As shown in FIG. 48, the two matching portions will encase the soft tissue graft on all sides. The relative position of each phase of the triphasic scaffold would be in the anatomical position, i.e., with Phase A (soft tissue) exposed to the joint cavity, Phase B (fibrocartilage interface) flush with articular cartilage, and Phase C (bone) encased within the bone tunnel. There are several advantages to this novel interference screw design: 1) the biomimetic triphasic screw design enables the regeneration of the relevant tissue types on the scaffold system, 2) the partitioned design permits the application of mechanical loading to the graft, which has been known to induce fibrocartilage formation, and 3) the tendon graft is in contact with the triphasic scaffold on all sides. Any applied mechanical and chemical stimulation would be uniformly experienced by the graft.

The optimal outcome scenario post-degradation of the screw or graft collar is to have a completely mineralized tissue within the bone tunnel, accompanied by the formation of a physiologically equivalent fibrocartilage insertion directly outside the bone.

For ligament tissue engineering, the triphasic scaffold may be coupled with synthetic grafts for ACL replacement. The future design of ACL replacement grafts must take into consideration the integration of the graft with bone. In this integrative ACL prosthesis design, the ACL prosthesis will contain three regions, a bony end consisting of Phase C, followed by Phase B, then by polymer fiber-based ACL portion. The triphasic scaffold can also be incorporated into any existing ACL prosthesis design, as the soft tissue graft shown in FIGS. 26A-B and FIG. 48 can easily be replaced by any synthetic ACL reconstruction scaffold. For example, in the case of a degradable polymer-based ACL prosthesis (Cooper, 2005), the triphasic scaffold can be sintered onto the polymer scaffold and implanted for ACL reconstruction.

One common feature in the above examples of clinical application is the focus on engineering soft tissue-to-bone integration ex vivo, which would reduce the complexity of graft reconstruction to just bone-to-bone integration in vivo. This is more feasible clinically as it is much more difficult to integrate soft tissue with bone compared to bone-to-bone integration.

The described approach is based on characterization of the chemical and mechanical properties of the interface (Wang, 2006; Spalazzi, 2006; Moffa, 2005), novel in vitro co-culture (Wang, 2005) and tri-culture (Wang 2006) models which have been developed to examine the role of cell-cell interactions in interface formation, and the knowledge of in vivo models of tendon-bone healing (Rodeo, 1993; Kawamura, 2005).

However, this approach is unique in that previous tissue engineering methods have focused predominantly on the design of a single type of tissue (e.g., only ligament or bone) on a scaffold with uniform properties. Moreover, the novel scaffold design and co-culture methods described here can be applied to treat other clinical conditions (e.g., rotator cuff, osteoarthritis) and will enable the design of a new generation of integrative fixation devices. The described studies will also provide fundamental insights into the mechanism of soft tissue-bone interface regeneration.

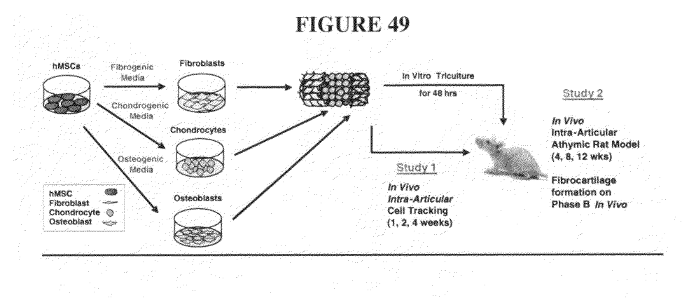

Clinical feasibility of the scaffold was determined by testing the hypothesis that the biomimetic matrix heterogeneity engineered on the triphasic scaffold will be maintained in vivo in an intra-articular model. A summary schematic of this research approach is presented below in FIG. 49. It was determined that modifications to the scaffold design were necessary to achieve distinct cell and matrix regions in vivo.

### Scaffold Design Optimization

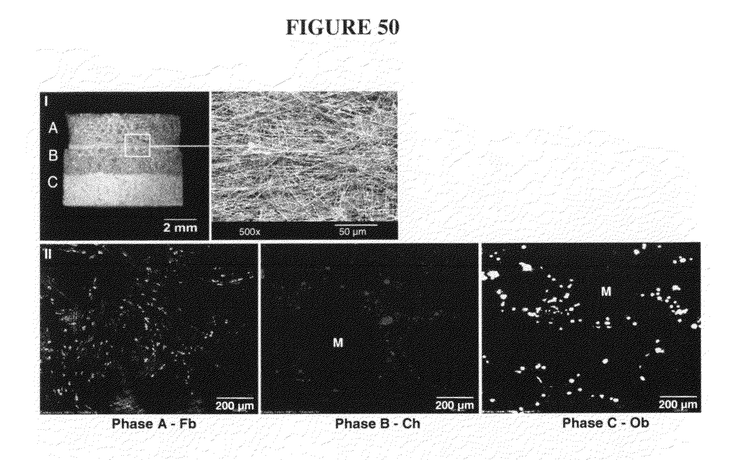

Based on the outcomes of in vitro and in vivo co-culture and tri-culture experiments, the multi-phased scaffold design has been improved upon, with the goal of localizing the interface-relevant cells within Phase B without compromising the scaffold design requirements (higher porosity and pore diameter) necessary for Phase A. Specifically, a degradable cell barrier between adjacent phases has been incorporated. This barrier is based on a polylactide-co-glycolide (PLGA) electrospun nanofiber mesh (FIG. 50-I), which, based on porosimetry analysis, has an average pore diameter of 5.2±0.9 μm. This nanofiber mesh will prevent unwanted cell migration and gel infiltration into Phase A or Phase C. Cell localization is important as 3-D co-culture results demonstrate that cell-specific distribution is required for the development of the biomimetic, controlled matrix distribution on the multi-phased scaffold.

Preliminary cell tracking results of fibroblasts and osteoblasts tri-cultured with chondrocytes loaded in hydrogel for 24 hours on the modified scaffold are shown in FIG. 50-II. Fibroblasts, chondrocytes, and osteoblasts were detected only in their respective phases as determined by fluorescence confocal microscopy. The nanofiber mesh served as an effective barrier to gel infiltration and unwanted cell cross-migration. It is anticipated that the mesh will degrade over time, having ensured the establishment of cell-specific regions in tri-

Mesenchymal Stem Cells and Differentiation into Interface-Relevant Cell Populations

The experiments will also utilize fibroblasts, chondrocytes, and osteoblasts derived from adult mesenchymal stem cells (MSCs) originated from human bone marrow. The MSCs are chosen because they are ideal for tissue engineering applications. These cells can be harvested from the patient prior to surgery, expanded, and pre-differentiated into desired cell types, and then seeded onto 3-D scaffolds. In addition to being autologous, MSCs can differentiate into fibroblasts Pittenger, 1999, Moreau, 2005), chondrocytes (Pittenger, 1999; Meinel, 2004), and osteoblasts (Pittenger, 1999; Mauney, 2005) which are the relevant cell types found at the soft tissue-bone interface. This versatility will simplify the tissue harvest process to a single procedure instead of the normal three required to obtain the three types of cells. Successful implementation of MSC-derived cells will significantly enhance the clinical feasibility and translational potential of the triphasic scaffold.

Specifically, MSCs purchased from Cambrex will be pre-differentiated into fibroblasts (Fb), chondrocytes (Ch), and osteoblasts (Ob) based on well-established protocols. The fibrogenic media will contain 1 ng/mL of basic fibroblast growth factor, 5 ng/mL of transforming growth factor-beta (TGF-β3) and 50 μg/ml of L-Ascorbic Acid-2-Phosphate (AA) (Moreau, 2005; Altman, 2002). The chondrogenic media will contain 5 ng/mL TGF-β3, 0.1 mM non-essential amino acids, 50 μg/ml AA, 10 nM dexamethasone (Dex), and 5 μg/ml of insulin16]. The osteogenic media will contain 10 nM Dex, 10 mM of β-glycerophosphate, and 50 μg/ml AA (Mauney, 2005).

### Intra-Articular ACL Reconstruction Model

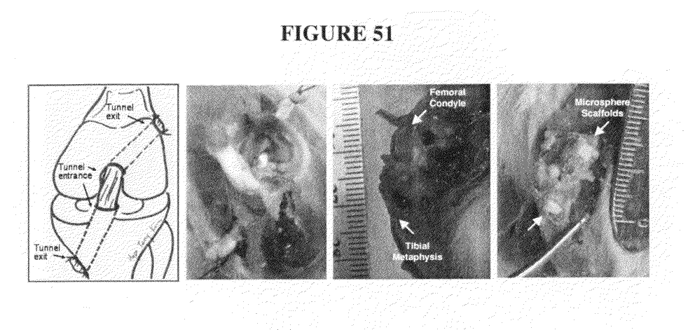

The study will use male athymic rats (Charles River Laboratories, mean weight 300 grams) to demonstrate unilateral ACL reconstruction (Rodeo, 2006) using a flexor digitorum longus tendon graft from the ipsilateral limb, as shown in FIG. 51-I. The rats will be anesthetized with a mixture of ketamine hydrochloride 80 mg/kg and xylazine 5 mg/kg, administered intraperitoneally. Ampicillin 25 mg/kg subcutaneous injection will be used for antibiotic prophylaxis. After appropriate anesthesia, the rat will be prepared for sterile surgery. The flexor digitorum longus tendon will be harvested via a longitudinal incision made on the medial aspect of the distal leg and ankle. The full length of the flexor digitorum longus tendon (average length 20 mm) will be harvested. An incision will be made over the rat knee, and a lateral parapatellar arthrotomy will be performed. The ACL, PCL, MCL, and LCL will be excised. Sectioning these ligaments causes minimal trauma to the knee and is not expected to affect the overall biologic response that will already occur from the knee arthrotomy. Using a needle with outer diameter of 2.5 mm, a bone tunnel will be made in the proximal tibia and the distal femur, entering the joint at the attachment sites of the ACL. We will measure the total length of the femur-tendon-tibia complex to determine the amount of displacement required to apply 1% and 10% strain.

The triphasic scaffold fabricated in the form of the graft collar will be used for implantation. After incorporating the graft collar onto the flexor tendon graft, the graft-scaffold complex will be passed through the bone tunnels to replace the ACL. Both ends of the grafted tendon will be secured to the surrounding periosteum at the extra-articular tunnel exit sites at the distal femur and well as proximal tibia using 4-0 Ethibond suture. Post-operative activity will be controlled using an external fixator that we have designed and fabricated for rat knees (Rodeo, 2006).

### Cell Tracking In Vivo

A further objective of these experiments is to track the three types of implanted cell populations in vivo and to determine their presence over a 4-week implantation period.

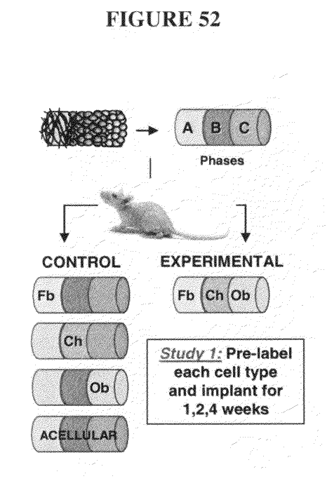

Cell Labeling—After pre-differentiation of MSCs into Fb, Ch, and Ob, cells will be seeded based on the optimal cell seeding density (cells/cm³) on their designated phase of the triphasic scaffold based on results from Phase I. As shown in FIG. 50, the Fb will be pre-labeled with Vybrant DiD dye (green), Ch with Vybrant DiO (red), and Ob with Vybrant Dil (yellow). All dyes can be purchased from Molecular Probes. The pre-label cells will be seeded on their respective phases of the triphasic scaffold collar, and tricultured for 2 days following established protocols (Spalazzi, 2006). As summarized in FIG. 52, the scaffold (n=3 per group) will be implanted for 1, 2, and 4 weeks, and the presence of the cells will be tracked over time and correlated to the formation of fibrocartilage tissue on the triphasic scaffold. At each time point, the scaffold collar+graft complex will be excised and cryosectioned for fluorescence microscopy (cell imaging) and histological analysis (fibrocartilage formation). Specifically, development of interface-relevant markers will be determined: proteoglycan and mineral deposition, as well as immunohistochemistry for collagen types I, II, III, IX, and X. Acellular scaffolds and unoperated contralateral insertion sites will serve as additional controls. A total of 45 animals (15 per time point) will be needed for this experiment.

### In Vivo Evaluation for Interface Regeneration

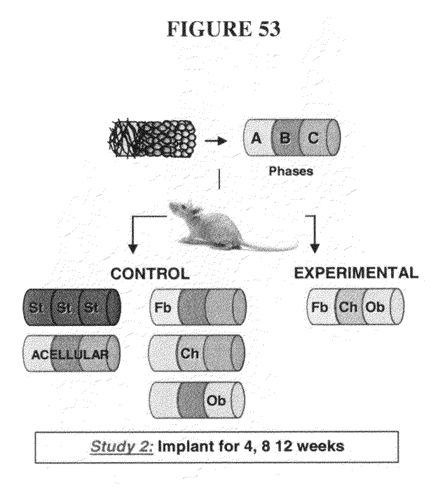

This experiment further focuses on interface regeneration on the tri-cultured, triphasic scaffold in an intra-articular ACL reconstruction model. Specifically, MSC-derived fibroblasts, chondrocytes and osteoblasts will be seeded on their respective phases of the triphasic scaffold, and cultured in vitro for 2 days (Spalazzi, 2006). The scaffold will be implanted following the methods described in Section E.3 and the experimental design outlined in FIG. 53. Each animal will receive one scaffold (randomly selected) and will be sacrificed at 4, 8, and 12 weeks. Outcomes will be evaluated using histomorphometric, micro-CT, and biomechanical analyses. Quantitative histomorphometric measurements will be made using the Bioquant Image Analysis system (R&M Biometrics, Inc., Nashville, Tenn.) available in the Analytical Microscopy Laboratory (Director, Dr. S. Doty). The implant evaluation methods successfully utilized in the previously described in vivo studies will also be used here. Specifically, the development of a fibrocartilage-like tissue and interfacial markers (n=3, see Section E.4) will be determine. Scaffold mechanical properties (n=6) will also be determined over time. Mineralization (total bone mineral content, bone volume fraction, and mineral distribution) will be analyzed by micro-CT prior to mechanical testing, so an additional sample is not needed. A push-out test (Knowles, 1992) will be performed on week 12 samples (tri-culture only, n=6) in order to determine the osteointegration potential of Phase C within the bone tunnel. A total of 168 male athymic nude rats (54 animals each for weeks 4 & 8, and 60 animals for week 12) will be used in this experiment.

### Expected Outcomes

It is anticipated that for the in vivo cell tracking experiment, all three cell types will persist at the implantation site for up to 4 weeks, and that the seeded chondrocytes will contribute to the formation of a fibrocartilage-like region on the interface phase (Phase B) of the triphasic scaffold. For the in vivo evaluation of interface regeneration experiment, it is expected that an interface-like region will form on the scaffold post-ACL reconstruction.

In these experiments, the formation of a fibrocartilage-like tissue on the interface phase of the triphasic scaffold has been focused on for several reasons. The long term role of the scaffold as a graft collar is to induce fibrocartilage formation on the reconstructed graft. After establishing the stability of the triphasic scaffold in the intra-articular model, and the viability of application of controlled mechanical stimulation to induce fibrocartilage formation on the graft, the next stage of the project will focus on the application of controlled chemical stimulation to induce fibrocartilage formation on the graft. For example, phase-specific growth factor delivery can be incorporated to provide chemical stimuli for interface regeneration. It is however critical to first establish the feasibility of the tri-culture, triphasic scaffold in a physiologically relevant intra-articular model.

### REFERENCES

1. Abate, J A, Fadale, P D, Hulstyn, M J & Walsh, W R, (1998) “Initial fixation strength of polylactic acid interference screws in anterior cruciate ligament reconstruction” Arthroscopy 14:278-284.
2. Allum, R L, (2001) “BASK Instructional Lecture 1: graft selection in anterior cruciate ligament reconstruction,” Knee 8:69-72.
3. Altman, G H, et al. (2002) “Advanced bioreactor with controlled application of multi-dimensional strain for tissue engineering” Journal of Biomechanical Engineering 124:742-749.
4. Altman, G H, et al., (2002) “Silk matrix for tissue engineered anterior cruciate ligaments” Biomaterials 23:4131-4141.
5. American Academy of Orthopaedic Surgeons, (1997) “Arthoplasty and Total Joint Replacement Procedures: United States 1990 to 1997” (Report, United States).
6. Anderson, K, Seneviratne, A M, Izawa, K, et al, (2001) “Augmentation of tendon healing in an intraarticular bone tunnel with use of a bone growth factor” Am. J. Sports Med. 29:689-698.
7. Batra, G S, Harrison, J W, Clough, T M, and Paul, A S (2002) “Failure of anterior cruciate ligament reconstruction following calcification of the graft” Knee 9:245-247.
8. Batycky, R P, Hanes, J, Langer, R & Edwards, D A, (1997) “A theoretical model of erosion and macromolecular drug release from biodegrading microspheres, J. Pharmaceutical Sciences 86:1464-1477.
9. Bellincampi, L D, Closkey, R F, Prasad, R, Zawadsky, J P & Dunn, M G, (1998) “Viability of fibroblast-seeded ligament analogs after autogenous implantation” J. Orthop. Res 16:414-420.
10. Benjamin, M and Ralphs, J R (1998) “Fibrocartilage in tendons and ligaments an adaptation to compressive load” Anat. 193(Pt 4):481-494.
11. Benjamin, M, Evans, E J, and Copp, L (1986) “The histology of tendon attachments to bone in man” J Anat 149:89-100.
12. Benjamin, M, Evans, E J, Rao, R D, Findlay, J A & Pemberton, D J, (1991) “Quantitative differences in the histology of the attachment zones of the meniscal horns in the knee joint of man” J. Anat. 177:127-134.
13. Berg, E E, (1996) “Autograft bone-patella tendon-bone plug comminution with loss of ligament fixation and stability” Arthroscopy 12:232-235.
14. Beynnon B D et al., (2002) “Anterior Cruciate Ligament Replacement: Comparison of Bone-Patellar Tendon-Bone Grafts with Two-Strand Hamstring Grafts: A Prospective, Randomized Study” J Bone Joint Surg Am 84-A:1503-1513, 2002.
15. Beynnon B D et al., (1996) “A sagittal plane model of the knee and cruciate ligaments with application of a sensitivity analysis” J. Biomech. Eng. 118:227-239.
16. Beynnon B D et al., (1997) “The effect of functional knee bracing on the anterior cruciate ligament in the weightbearing and nonweightbearing knee” Am. J. Sports Med. 25:353-359.
17. Blickenstaff, K R, Grana, W A & Egle, D, (1997) “Analysis of a semitendinosus autograft in a rabbit model” Am. J. Sports Med. 25:554-559.
18. Bolton, C W & Bruchman, W C, (1985) “The GORE-TEX expanded polytetrafluoroethylene prosthetic ligament. An in vitro and in vivo evaluation” Clin. Orthop. 202-213.
19. Bonfield, W, (1988) “Composites for bone replacement,” J. Biomed. Eng. 10:522-526 (1988).
20. Borden, M, Attawia, M, Khan, Y & Laurencin, C T, (2002) “Tissue engineered microsphere-based matrices for bone repair: design and evaluation” Biomaterials 23:551-559.
21. Boskey, A L, et al., (1996) “The mechanism of beta-glycerophosphate action in mineralizing chick limb-bud mesenchymal cell cultures” J. Bone Min. Res. 11:1694-1702.
22. Brand, J. Jr., Weiler, A, Caborn, D N, Brown, C H. Jr. & Johnson, D L, (2000) “Graft fixation in cruciate ligament reconstruction” Am. J. Sports Med. 28:761-774.
23. Brody, G A, Eisinger, M, Arnoczky, S P & Warren, R F, (1998) “In vitro fibroblast seeding of prosthetic anterior cruciate ligaments. A preliminary study” Am. J. Sports Med. 16:203-208.
24. Bromage, T G, Smolyar, I, Doty, S B, Holton, E & Zuyev, A N, (1998) “Bone growth rate and relative mineralization density during space flight” Scanning 20:238-239.
25. Burkart, A, Imhoff, A B & Roscher, E, (2000) “Foreign-body reaction to the bioabsorbable suretac device” Arthroscopy 16:91-95.
26. Butler, D L, Goldstein, S A & Guilak, F, (2000) “Functional tissue engineering: the role of biomechanics” J. Biomech. Eng. 122:570-575.
27. Chen, C H, et al., (2003) “Enveloping the tendon graft with periosteum to enhance tendon-bone healing in a bone tunnel: A biomechanical and histologic study in rabbits” Arthroscopy 19:290-296.
28. Chen, C S, Mrksich, M, Huang, S, Whitesides, G M, and Ingber, D E (1997) “Geometric control of cell life and death” Science 276:1425-1428.
29. Cheung, H S & McCarty, D J, (1985) “Mitogenesis induced by calcium-containing crystals. Role of intracellular dissolution” Exp. Cell Res. 157:63-70.
30. Clark, J M & Sidles, J A, (199) “The interrelation of fiber bundles in the anterior cruciate ligament” J. Orthop. Res. 8:180-188).
31. Cooper J A, et al., (2005) “Fiber-based tissue-engineered scaffold for ligament replacement: design considerations and in vitro evaluation” Biomaterials, 26(13):1523-1532.
32. Cooper, J A, (2002) “Design, optimization and in vivo evaluation of a tissue-engineered anterior cruciate ligament replacement,” Drexel University (Thesis/Dissertation).
33. Cooper, J A, Lu, H H & Laurencin, C T, (2000) “Fiber-based tissue engineering scaffold for ligament replacement: design considerations and in vitro evaluation,” Proceedings of 5^{th} World Biomaterial Congress, 208 (Abstract).
34. Cooper, R R and Misol, S (1970) “Tendon and ligament insertion. A light and electron microscopic study” J Bone Joint Surg Am. 52:1-20, 1970.
35. Daniel, D M, et al., (1994) “Fate of the ACL-injured patient. A prospective outcome study” Am. J. Sports Med. 22:632-644.
36. Deitzel et al., (2001) Polymer 42.
37. Deitzel et al., (2002) Polymer 43.
38. Doshi, J and Reneker, D H (1995) “Electrospinning process and applications of electrospun fibers” Journal of Electrostatics 35:151-160.
39. Ducheyne, P I (1987) “Bioceramics: material characteristics versus in vivo behavior” J. Biomed. Matls. Res. 21:219-236 (1987).
40. Dunn, M G, Liesch, J B, Tiku, M L, Maxian, S H & Zawadsky, J P, (1994) “The Tissue Engineering Approach to Ligament Reconstruction,” Matls. Res. Soc. 331:13-18.
41. El-Amin, S F, et al., (2001) “Human osteoblast integrin expression on degradable polymeric materials for tissue engineered bone,” J. Orthop. Res. (In Press).
42. Erben, (1997) “Embedding of Bone Samples in Methylmethacrylate: An Improved Method Suitable for Bone Histomorphometry, Histochemistry, and Immunohistochemistry” J. Histochem. Cytochem. 45:307-314.
43. Eriksson, K, Kindblom, L G, and Wredmark, T, (2000) “Semitendinosus tendon graft ingrowth in tibial tunnel following ACL reconstruction: a histological study of 2 patients with different types of early graft failure” Acta Orthop. Scand. 71:275-279.
44. Evanko, S P and Vogel, K G (1993) “Proteoglycan synthesis in fetal tendon is differentially regulated by cyclic compression in vitro” Arch. Biochem. Biophys. 307:153-164.
45. Evanko, S P and Vogel, K G (1993) “Proteoglycan synthesis in fetal tendon is differentially regulated by cyclic compression in vitro” Arch. Biochem. Biophys. 307:153-164.
46. Fleming, B, Beynnon, B, Howe, J, McLeod, W & Pope, M, (1992) “Effect of tension and placement of a prosthetic anterior cruciate ligament on the anteroposterior laxity of the knee” J. Orthop. Res. 10:177-186.
47. Fleming, B C, Abate, J A, Peura, G D & Beynnon, B D, (2001) “The relationship between graft tensioning and the anterior-posterior laxity in the anterior cruciate ligament reconstructed goat knee” J. Orthop. Res. 19, 841-844.
48. Fong et al., (1999) “Beaded nanofibers formed during electrospinning” Polymer, 40:4583-4592.
49. Fridrikh et al., (2003) “Controlling the Fiber Diameter during Electrospinning” Phys. Rev. Lett. 90:144502.
50. Friedman, M J, Sherman, O H, Fox, J M, et al, (1985) “Autogeneic anterior cruciate ligament (ACL) anterior reconstruction of the knee. A review” Clinical Orthopaedics & Related Research. 196:9-14.
51. Fu, F H, Bennett, C H, Ma, C B, Menetrey, J & Lattermann, C, (2000) “Current trends in anterior cruciate ligament reconstruction. Part II. Operative procedures and clinical correlations” Am. J. Sports Med. 28:124-130.
52. Fujikawa, K, Iseki, F & Seedhom, B B, (1989) “Arthroscopy after anterior cruciate reconstruction with the Leeds-Keio ligament” J. Bone Joint Surg. Br. 71:566-570.
53. Gao, J & Messner, K, (1996) “Quantitative comparison of soft tissue-bone interface at chondral ligament insertions in the rabbit knee joint” J. Anat. 188:367-373.
54. Gao, J, Rasanen, T, Persliden, J & Messner, K, (1996) “The morphology of ligament insertions after failure at low strain velocity: an evaluation of ligament entheses in the rabbit knee” J. Anat. 189:127-133.
55. Gao, J, Messner, K, Ralphs, J R, and Benjamin, M, (1996) “An immunohistochemical study of enthesis development in the medial collateral ligament of the rat knee joint” Anat. Embryol. (Ber1) 194:399-406.
56. Goldblatt, J P, Fitzsimmons, S E, Balk, E, & Richmond, J C (2005) “Reconstruction of the anterior cruciate ligament: meta-analysis of patellar tendon versus hamstring tendon autograft” Arthroscopy 21:791-803.
57. Gotlin, R S & Huie, G, (2000) “Anterior cruciate ligament injuries. Operative and rehabilitative options” Phys. Med. Rehabil. Clin. N. Am. 11:895-928.
58. Goulet, F, et al., (2000) “Principles of Tissue Engineering,” Lanza, R. P., Langer, R. & Vacanti, J. P. (eds.), pp. 639-645 Academic Press.
59. Grana, W A, EgIe, D M, Mahnken, R, and Goodhart, C W (1994) “An analysis of autograft fixation after anterior cruciate ligament reconstruction in a rabbit model” Am J Sports Med 22:344-351.
60. Gregoire, M, Orly, I, Kerebel, L M & Kerebel, B, (1987) “In vitro effects of calcium phosphate biomaterials on fibroblastic cell behavior” Biol. Cell 59:255-260.
61. Gregor, R J and Abelew, T A (1994) “Tendon force measurements and movement control: a review” Med. Sci. Sports Exerc. 26:1359-1372.
62. Grossman, M G, ElAttrache, N S, Shields, C L, and Glousman, R E (2005) “Revision anterior cruciate ligament reconstruction: three- to nine-year follow-up” Arthroscopy 21:418-423.
63. Harner, C. D. et al., “Quantitative analysis of human cruciate ligament insertions,” Arthroscopy 15, 741-749 (1999).
64. Hench, L L, (1991) “Bioceramics: from concept to clinic” J. Am. Cera. Soc. 74(7):1487-1510.
65. Hiss J, Hirshberg A, Dayan D F, Bubis J J, Wolman M. (1988) “Aging of wound healing in an experimental model in mice” Am J Forensic Med. Pathol. 9:310-312.
66. Indelli, P F, Dillingham, M F, Fanton, G S, and Schurman, D J (2004) “Anterior cruciate ligament reconstruction using cryopreserved allografts” Clin. Orthop. Relat Res. 268-275.
67. Indelli, P F, Dillingham, M F, Fanton, G S, and Schurman, D J (2004) “Anterior cruciate ligament reconstruction using cryopreserved allografts” Clin. Orthop. Relat Res. 268-275.
68. Jackson, D W et al., (1993) “A comparison of patellar tendon autograft and allograft used for anterior cruciate ligament reconstruction in the goat model” Am. J. Sports Med. 21:176-185.
69. Jackson, D W, et al., (1991) Trans. Orhtop. Res. Soc. 16:208 (Abstract).
70. Jackson, D W, American Academy of Orthopaedic Surgeon Bulletin 40:10-11 (1992).
71. Jackson, D W, Grood, E S, Arnoczky, S P, Butler, D L & Simon, T M, (1987) “Cruciate reconstruction using freeze dried anterior cruciate ligament allograft and a ligament augmentation device (LAD). An experimental study in a goat model” Am. J. Sports Med. 15:528-538.
72. Jiang, J, Leong, N L, Mung, J C, Hidaka, C, and Lu, H H (2007) “Interaction between zonal populations of articular chondrocytes suppresses chondrocyte mineralization and this process is mediated by PTHrP. Osteoarthritis and Cartilage” In press.
73. Jiang, J, Nicoll, S B, and Lu, H H (2005) “Co-culture of osteoblasts and chondrocytes modulates cellular differentiation in vitro” Biochem. Biophys. Res. Commun. 338:762-770.
74. Jiang, J, Nicoll, S B & Lu, H H, (2003) “Effects of Osteoblast and Chondrocyte Co-Culture on Chondrogenic and Osteoblastic Phenotype In vitro” Trans. Orhtop. Res. Soc. 49 (Abstract) (2003).
75. Johnson, D H (2003) “Should allografts be used for routine anterior cruciate ligament reconstructions? No, allografts should not be used for routine ACL reconstruction” Arthroscopy 19:424-425.
76. Johnson, R J (1982) “The anterior cruciate: a dilemma in sports medicine” Int. J Sports Med. 3:71-79.
77. Junqueira, L C, Montes G S, Sanchez E M (1982) “The influence of tissue section thickness on the study of collagen by the Picrosirius-polarization method” Histochemistry. 74:153-156.
78. Kawamura S et al. (2005) ORS.
79. Kim et al., (2003) Biomaterials.
80. Knowles J C et al. (1992) “Development of a degradable composite for orthopaedic use: in vivo biomechanical and histological evaluation of two bioactive degradable composites based on the polyhydroxybutyrate polymer” Biomaterials 13(8):491-496.
81. Koob, T J, Clark, P E, Hernandez, D J, Thurmond, F A, & Vogel, K G (1992) “Compression loading in vitro regulates proteoglycan synthesis by tendon fibrocartilage” Arch. Biochem. Biophys. 298:303-312.
82. Kurosaka, M., Yoshiya, S & Andrish, J T, (1987) “A biomechanical comparison of different surgical techniques of graft fixation in anterior cruciate ligament reconstruction” Am. J. Sports Med. 15:225-229.
83. Kurzweil, P R, Frogameni, A D & Jackson, D W, (1995) “Tibial interference screw removal following anterior cruciate ligament reconstruction” Arthroscopy 11:289-291.
84. Larson, R P, (1994) “The Crucial Ligaments: Diagnosis and Treatment of Ligamentous Injuries About the Knee” John, A. Jr. & Feagin, J. A. (eds.), pp. 785-796 Churchill Livingstone, N.Y.
85. Li, K W, Lindsey, D P, Wagner, D R, et al, (2006) “Gene regulation ex vivo within a wrap-around tendon” Tissue Eng 12:2611-2618.
86. Li, W J, Laurencin, C T, Caterson, E J, Tuan, R S, & Ko, F K (2002) “Electrospun nanofibrous structure: a novel scaffold for tissue engineering” J. Biomed. Mater. Res. 60:613-621.
87. Liu, S H et al., (1997) “Morphology and matrix composition during early tendon to bone healing” Clinical Orthopaedics & Related. Research. 253-260.
88. Loh, J C, al., (2003) “Knee stability and graft function following anterior cruciate ligament reconstruction: Comparison between 11 o'clock and 10 o'clock femoral tunnel placement” Arthroscopy 19:297-304.
89. Lu, H H, et al., (2003) “Evaluation of Optimal Parameters in the Co-Culture of Human Anterior Cruciate Ligament Fibroblasts and Osteoblasts for Interface Tissue Engineering,” ASME 2003 Summer Bioengineering Conference (Abstract).
90. Lu, H H, Cooper, J A, Ko, F A, Attawia, M A & Laurencin, C T, (2001) “Effect of polymer scaffold composition on the morphology and growth of anterior cruciate ligaments cells” Society for Biomaterials Proceedings (Abstract).
91. Lu, H H, El Amin, S F, Scott, K D & Laurencin, C T, (2003) “Three dimensional, bioactive, biodegradable, polymer bioactive glass composite scaffolds with improved mechanical properties support collagen synthesis and mineralization of human osteoblast-like cells in vitro” J. Biomed. Matls. Res. 64A:465-474.
92. Lu, H H, Pollack, S R & Ducheyne, P, (2001) “45S5 bioactive glass surface charge variations and the formation of a surface calcium phosphate layer in a solution containing fibronectin,” J. Biomed. Matls. Res. 54:454-461.
93. Lu, H H, Pollack, S R & Ducheyne, P, (2000) “Temporal zeta potential variations of 45S5 bioactive glass immersed in an electrolyte solution” J. Biomed. Matls. Res. 51:80-87.
94. Lu, H H and Jiang, J (2006) “Interface tissue engineering and the formulation of multiple-tissue systems” Adv. Biochem. Eng Biotechnol. 102:91-111.
95. Lu, H H, Tang, A, Oh, S C, Spalazzi, J P, and Dionisio, K, (2005) “Compositional effects on the formation of a calcium phosphate layer and the response of osteoblast-like cells on polymer-bioactive glass composites” Biomaterials 26:6323-6334.
96. Malaviya, P, Butler, D L, Boivin, G P, et al (2000) “An in vivo model for load-modulated remodeling in the rabbit flexor tendon” J Orthop Res 18:116-125.
97. Malinin, T I, Levitt, R L, Bashore, C, Temple, H T,& Mnaymneh, W (2002) “A study of retrieved allografts used to replace anterior cruciate ligaments” Arthroscopy 18:163-170.
98. Markolf, K L, et al., (2002) “Effects of femoral tunnel placement on knee laxity and forces in an anterior cruciate ligament graft” J. Orthop. Res. 20:1016-1024.
99. Matthews, L S, Soffer, S R, (1989) “Pitfalls in the use of interference screws for anterior cruciate ligament reconstruction: brief report” Arthroscopy 5:225-226.
100. Matyas, J R, Anton, M G, Shrive, N G & Frank, C B (1995) “Stress governs tissue phenotype at the femoral insertion of the rabbit MCL” J. Biomech. 28:147-157.
101. Mauney J R et al., Biomaterials 26:3173-3185, 2005.
102. McCarthy, D M, Tolin, B S, Schwendeman, L, Friedman, M J & Woo, S L, (1993) “The Anterior Cruciate Ligament: Current and Future Concepts” Douglas, W. & M D Jackson (eds.) Raven Press, New York.
103. McGuire, D A (2003) “Should allografts be used for routine anterior cruciate ligament reconstructions? Yes, allografts should be used in routine ACL reconstruction” Arthroscopy 19:421-424.
104. Meinel L et al., (2004) Biotechnol Bioeng 88:379-391.
105. Messner, K, (1997) “Postnatal development of the cruciate ligament insertions in the rat knee. Morphological evaluation and immunohistochemical study of collagens types I and II” Acta Anatomica 160:261-268.
106. MiIz, S, McNeilly, C, Putz, R, Ralphs, J R, & Benjamin, M (1998) “Fibrocartilages in the extensor tendons of the interphalangeal joints of human toes” Anat. Rec. 252:264-270.
107. Moffat K L et al., (2005) ISTL-V.
108. Moffat, K L, Spalazzi, J P, Doty, S B, Levine, W N, & Lu, H H (2007) “In vitro evaluation of biomimetic nanofiber-based scaffolds for rotator cuff repair” Society for Biomaterials Proceedings Paper 195”.
109. Moffat, K L, Sun, W S, Chahine, N O, et al. (2006) “Characterization of the Mechanical Properties and Mineral Distribution of the Anterior Cruciate Ligament-to-Bone Insertion Site” Proceedings of the IEEE Engineering in Medicine and Biology Society.
110. Moffat, K L, Sun, W S, Spalazzi, J P, et al. (2007) “Nanofiber alignment in biodegradable polymer scaffold directs attachment and matrix elaboration of human rotator cuff” fibroblasts. Trans. Orthop. Res. Soc. 32:Paper #349.
111. Moore, P B & Dedman, J R, (1982) “Calcium binding proteins and cellular regulation” Life Sci. 31:2937-2946.
112. Moreau J E et al., (2005) J Orthop Res 23:164-174.
113. Nawata, K, Minamizaki, T, Yamashita, Y, and Teshima, R (2002) “Development of the attachment zones in the rat anterior cruciate ligament: changes in the distributions of proliferating cells and fibrillar collagens during postnatal growth” J. Orthop. Res. 20:1339-1344, 2002.
114. Nicoll, S B, Wedrychowska, A, Smith, N R & Bhatnagar, R S, (2001) “Modulation of proteoglycan and collagen profiles in human dermal fibroblasts by high density micromass culture and treatment with lactic acid suggests change to a chondrogenic phenotype” Connect. Tissue Res. 42:59-69.
115. Niyibizi, C., Sagarrigo, V C, Gibson, G & Kavalkovich, K, (1996) “Identification and immunolocalization of type X collagen at the ligament-bone interface” Biochem. Biophys. Res. Commun. 222:584-589.
116. Noyes, F R & Barber-Westin, S D, (1996) “Revision anterior cruciate ligament surgery: experience from Cincinnati” Clin. Orthop. 116-129.
117. Noyes, F R, Mangine, R E & Barber, S, (1987) “Early knee motion after open and arthroscopic anterior cruciate ligament reconstruction” Am. J. Sports Med. 15:149-160.
118. Panni, A S, Milano, F, Lucania, L & Fabbriciani, C, (1997) “Graft healing after anterior cruciate ligament reconstruction in rabbits” Clin. Orthop. 203-212.
119. Pena, F, Grontvedt, T, Brown, G A, Aune, A K & Engebretsen, L, (1996) “Comparison of failure strength between metallic and absorbable interference screws. Influence of insertion torque, tunnel-bone block gap, bone mineral density, and interference” Am. J. Sports Med. 24:329-334.
120. Perez-Castro, A V and Vogel, K G (1999) “In situ expression of collagen and proteoglycan genes during development of fibrocartilage in bovine deep flexor tendon” J. Orthop. Res. 17:139-148.
121. Petersen, W & Tillmann, B, (1999) “Structure and vascularization of the cruciate ligaments of the human knee joint” Anat. Embryol. (Ber1) 200:325-334.
122. Peterson, R K, Shelton, W R, and Bomboy, A L (2001) “Allograft versus autograft patellar tendon anterior cruciate ligament reconstruction: A 5-year follow-up” Arthroscopy 17:9-13.
123. Pittenger M F et al., (1999) Science 284:143-147.
124. Poehling, G G, Curl, W W, Lee, C A, et al. (2005) “Analysis of outcomes of anterior cruciate ligament repair with 5-year follow-up: allograft versus autograft” Arthroscopy 21:774-785.
125. Rich L & Whittaker P, (2005) “Collagen and picrosirius red staining: a polarized light assessment of fibrillar hue and spatial distribution” Braz J Morphol Sci. 22:97-104.
126. Robbins, J R, Evanko, S P, and Vogel, K G (1997) “Mechanical loading and TGF-beta regulate proteoglycan synthesis in tendon” Arch. Biochem. Biophys. 342:203-211.
127. Robertson, D B, Daniel, D M & Biden, E, (1986) “Soft tissue fixation to bone” Am. J. Sports Med. 14:398-403.
128. Rodeo et al., (2006) ORS.
129. Rodeo, S A, Arnoczky, S P., Torzilli, P A, Hidaka, C & Warren, R F, (1993) “Tendon-healing in a bone tunnel. A biomechanical and histological study in the dog” J. Bone Joint Surg. Am. 75:1795-1803.
130. Rodeo, S A, Suzuki, K, Deng, X H, Wozney, J & Warren, R F, (1999) “Use of recombinant human bone morphogenetic protein-2 to enhance tendon healing in a bone tunnel” Am. J. Sports Med. 27:476-488.
131. Rodeo, S A (2001) “Studies of tendon-to-bone healing: exploring ways to improve graft fixation following anterior cruciate ligament reconstruction” Journal of Bone and Joint Surgery.
132. Safran, M R & Harner, C D, (1996) “Technical considerations of revision anterior cruciate ligament surgery” Clin. Orthop. 50-64.
133. Sagarriga, V C, Kavalkovich, K, Wu, J & Niyibizi, C, (1996) “Biochemical analysis of collagens at the ligament-bone interface reveals presence of cartilage-specific collagens” Arch. Biochem. Biophys. 328:135-142.
134. Scapinelli, R & Little, K, (1970) “Observations on the mechanically induced differentiation of cartilage from fibrous connective tissue” J. Pathol. 101:85-91.
135. Schafer, et al., (2000) “In vitro generation of osteochondral composites” Biomaterials 21:2599-2606.
136. Schafer, et al., (2002) “Tissue-engineered composites for the repair of large osteochondral defects” Arthritis Rheum. 46:2524-2534.
137. Shapiro, L and Cohen, S (1997) “Novel alginate sponges for cell culture and transplantation” Biomaterials 18:583-590.
138. Shellock, F G, Mink, J H, Curtin, S & Friedman, M J, (1992) “MR imaging and metallic implants for anterior cruciate ligament reconstruction: assessment of ferromagnetism and artifact” J. Magn. Reson. Imaging 2:225-228.
139. Shelton, W R, Papendick, L, and Dukes, A (1997) “Autograft versus allograft anterior cruciate ligament reconstruction” Arthroscopy 13:446-449.
140. Sherman, O H and Banffy, M B (2004) “Anterior cruciate ligament reconstruction: which graft is best?” Arthroscopy 20:974-980.
141. Shin et al., (2001) Polymer 42.
142. Sittinger, et al., (1994) “Engineering of cartilage tissue using bieresorbable polymer carriers in perusion culture” Biomaterials 15(6):451-456.
143. Song, E K, Rowe, S M, Chung, J Y, Moon, E S, and Lee, K B (2004) “Failure of osteointegration of hamstring tendon autograft after anterior cruciate ligament reconstruction” Arthroscopy 20:424-428.
144. Spalazzi J P et al., (2006) J Orthop Res 24(10):2001-2010.
145. Spalazzi, J P, Dionisio, K L, Jiang, J & Lu, H H, (2003) “Chondrocyte and Osteoblast Interaction on a Degradable Polymer Ceramic Scaffold” ASME 2003 Summer Bioengineering Conference (Abstract).
146. Spalazzi, J P, Dagher, E, Doty, S B, et al (2006) “In vivo Evaluation of a TriPhasic Composite Scaffold for Anterior Cruciate Ligament-to-Bone Integration” Proceedings of the IEEE Engineering Biology and Medicine Society.
147. Spalazzi, J P, Doty, S B, Moffat, K L, Levine, W N, and Lu, H H, (2006) “Development of Controlled Matrix Heterogeneity on a Triphasic Scaffold Orthopedic Interface Tissue Engineering” Tissue Engineering 12:3497-3508.
148. Spalazzi, J P, Gallina, J, Fung-Kee-Fung, S D, Konofagou, E E, and Lu, H H (2006) “Elastographic imaging of strain distribution in the anterior cruciate ligament and at the ligament-bone insertions” J. Orthop. Res DOI 10.100.jor.
149. Steiner, M E, Hecker, A T, Brown, C H, Jr. & Hayes, W C, (1994) “Anterior cruciate ligament graft fixation. Comparison of hamstring and patellar tendon grafts” Am. J. Sports Med. 22:240-246.
150. Thomas, N P, Turner, I G & Jones, C B, (1987) “Prosthetic anterior cruciate ligaments in the rabbit. A comparison of four types of replacement” J. Bone Joint Surg. Br. 69:312-316.
151. Thomopoulos, S. et al., (2002) “The localized expression of extracellular matrix components in healing tendon insertion sites: an in situ hybridization study” J. Orthop. Res. 20:454-463.
152. Vanderploeg, E J, Imler, S M, Brodkin, K R, Garcia, A J, & Levenston, M E (2004) “Oscillatory tension differentially modulates matrix metabolism and cytoskeletal organization in chondrocytes and fibrochondrocytes” J. Biomech. 37:1941-1952.
153. Vogel, K G and Koob, T J (1989) “Structural specialization in tendons under compression” Int. Rev. Cytol. 115:267-293.
154. Vogel, K G (1996) “The effect of compressive loading on proteoglycan turnover in cultured fetal tendon” Connect. Tissue Res. 34:227-237.
155. Wagner, M, Kaab, M J, Schallock, J, Haas, N P, and Weiler, A (2005) “Hamstring tendon versus patellar tendon anterior cruciate ligament reconstruction using biodegradable interference fit fixation: a prospective matched-group analysis” Am. J. Sports Med. 33:1327-1336.
156. Wang I E and Lu H H, (2005) ORS.
157. Wang, I N, Mitroo, S, Chen, F H, Lu, H H, and Doty, S B (2006) Age-dependent changes in matrix composition and organization at the ligament-to-bone insertion” J. Orthop. Res. 24(8):1745-1755.
158. Wei, X & Messner, K, (1996) “The postnatal development of the insertions of the medial collateral ligament in the rat knee” Anat. Embryol. (Ber1) 193:53-59.
159. Weiler, A, Hoffmann, R F, Bail, H J, Rehm, O & Sudkamp, N P, (2002) “Tendon healing in a bone tunnel. Part II: Histologic analysis after biodegradable interference fit fixation in a model of anterior cruciate ligament reconstruction in sheep” Arthroscopy 18:124-135.
160. Weiler, A, Windhagen, H J, Raschke, M J, Laumeyer, A & Hoffmann, R F, (1998) “Biodegradable interference screw fixation exhibits pull-out force and stiffness similar to titanium screws” Am. J. Sports Med. 26:119-126.
161. Weiler, A, Peine, R, Pashmineh-Azar, A, et al (2002) “Tendon healing in a bone tunnel. Part I: Biomechanical results after biodegradable interference fit fixation in a model of anterior cruciate ligament reconstruction in sheep” Arthroscopy. 18:113-123.
162. Woo, S L, Newton, P O, MacKenna, D A & Lyon, R M, (1992) “A comparative evaluation of the mechanical properties of the rabbit medial collateral and anterior cruciate ligaments” J. Biomech. 25:377-386.
163. Woo, S L and Buckwalter, J A (1988,) AAOS/NIH/ORS workshop, “Injury and repair of the musculoskeletal soft tissues” Savannah, Ga., Jun. 18-20, 1987. J. Orthop. Res. 6:907-931.
164. Woo, S L, Gomez, M A, Seguchi, Y, Endo, C M, and Akeson, W H (1983) “Measurement of mechanical properties of ligament substance from a bone-ligament-bone preparation” J. Orthop. Res. 1:22-29.
165. Wu H, et al., (2003) J. Am. Chem. Soc. 2003 Jan. 15; 125(2):554-559.
166. Wuthier, R E, (1993) “Involvement of cellular metabolism of calcium and phosphate in calcification of avian growth plate cartilage” J. Nutr. 123:301-309.
167. Xu et al., (2004) Biomaterials 25, 877-886.
168. Yahia, L, (1997) “Ligaments and Ligamentoplasties,” Springer Verlag, Berlin Heidelberg.
169. Yang, F, Murugan, R, Wang, S, and Ramakrishna, S (2005) “Electrospinning of nano/micro scale poly(L-lactic acid) aligned fibers and their potential in neural tissue engineering” Biomaterials 26:2603-2610.
170. Yoshiya, S, Nagano, M, Kurosaka, M, Muratsu, H & Mizuno, K, (2000) “Graft healing in the bone tunnel in anterior cruciate ligament reconstruction” Clin. Orthop. 278-286.
171. Zong, X, Ran, S, Kim, K S, et al. (2003) “Structure and Morphology Changes during in vitro Degradation of Electrospun Poly(glycolide-co-lactide) Nanofiber Membrane” Biomacromolecules. 4:416-423.

What is claimed is:

1. A graft collar for fixing tendon to bone in a subject, wherein said graft collar comprises a sheet of biopolymer mesh or polymer-fiber mesh.

2. The graft collar of claim 1, wherein the biopolymer mesh or polymer-fiber mesh comprises aligned fibers.

3. The graft collar of claim 1, wherein the biopolymer mesh or polymer-fiber mesh comprises unaligned fibers.

4. The graft collar of claim 1, wherein the graft collar comprises a sheet of biopolymer mesh and the biopolymer mesh is derived from at least one of collagen, chitosan, silk and alginate.

5. The graft collar of claim 1, wherein the graft collar comprises a sheet of biopolymer mesh and the biopolymer mesh is allogeneic or xenogenic.

6. The graft collar of claim 1, wherein the graft collar comprises a sheet of polymer-fiber mesh and the polymer-fiber mesh comprises aliphatic polyesters, poly(amino acids), copoly(ether-esters), polyalkylenes oxalates, polyamides, poly(iminocarbonates), polyorthoesters, polyoxaesters, polyamidoesters, poly(ε-caprolactone)s, polyanhydrides, polyarylates, polyphosphazenes, polyhydroxyalkanoates, polysaccharides, biopolymers, poly(lactic-co-glycolic acid), poly(lactide), poly(glycolide) or a blend of two or more of the preceding polymers.

7. The graft collar of claim 1, wherein the polymer-fiber mesh comprises at least one of poly(lactide-co-glycolide), poly(lactide) or poly(glycolide).

8. The graft collar of claim 1, wherein the graft collar is sutured around a tendon graft.

9. The graft collar of claim 8, wherein the tendon graft is a bone-patellar tendon-bone (BPTB) graft, a patellar tendon graft, a semitendinosus, a hamstring-tendon (HST) graft, a hamstring tendon graft, a quadriceps tendon graft, Achilles graft or tibialis graft.

10. The graft collar of claim 8, wherein the tendon graft is an allograft or an autograft.

11. The graft collar of claim 1, wherein the subject is a mammal.

12. The graft collar of claim 11, wherein the mammal is a human.

13. The graft collar of claim 8, wherein the graft collar promotes integration of the tendon graft to bone.

14. The graft collar of claim 1, wherein the graft collar includes at least one of the following substances: anti-infectives, antibiotics, bisphosphonate, hormones, analgesics, anti-inflammatory agents, growth factors, angiogenic factors, chemotherapeutic agents, anti-rejection agents, and RGD peptides.

15. The graft collar of claim 14, wherein the growth factors are selected from the group consisting of TGFs, BMPS, IGFS, VEGFs and PDGFS.

16. The graft collar of claim 15, wherein the TGF is TGF-β.

17. The graft collar of claim 15, wherein the BMP is BMP-2.

18. The graft collar of claim 1, wherein the graft collar includes one or more of the following types of cells: chondrocytes, osteoblasts, osteoblast-like cells and stem cells.

19. The graft collar of claim 1, wherein the graft collar includes at least one of the following: osteogenic agents, osteogenic materials, osteoinductive agents, osteoinductive materials, osteoconductive agents, osteoconductive materials and chemical factors.

20. The graft collar of claim 1, wherein the graft collar promotes regeneration of an interfacial region between tendon and bone.

21. The graft collar of claim 1, wherein the graft collar is lyophilized.

22. The graft collar of claim 1, wherein the graft collar is biodegradable.

23. The graft collar of claim 1, wherein the graft collar is osteointegrative.

24. A graft collar for fixing tendon to bone in a subject, wherein the graft collar comprises:
a) a first region comprising a biopolymer mesh or a polymer-fiber mesh and hydrogel; and
b) a second region adjoining the first region and comprising a biopolymer mesh or a polymer-fiber mesh,
wherein the mesh in the first region and the mesh in the second region are different from each other.

25. The graft collar of claim 24, wherein the subject is a mammal.

26. The graft collar of claim 25, wherein the mammal is a human.

27. The graft collar of claim 24, wherein the first region supports the growth and maintenance of an interfacial zone between tendon and bone, and the second region supports the growth and maintenance of bone tissue.

28. The graft collar of claim 24, wherein the graft collar includes at least one of the following substances: anti-infectives, antibiotics, bisphosphonate, hormones, analgesics, antiinflammatory agents, growth factors, angiogenic factors, chemotherapeutic agents, anti-rejections agents, and RGD peptides.

29. The graft collar of claim 24, wherein the hydrogel is photopolymerized, thermoset or chemically cross-linked.

30. The graft collar of claim 29, wherein the hydrogel is polyethylene glycol.

31. The graft collar of claim 24, wherein the biopolymer mesh comprises aligned fibers.

32. The graft collar of claim 24, wherein the biopolymer mesh comprises unaligned fibers.

33. The graft collar of claim 24, wherein the first region contains TGF.

34. The graft collar of claim 33, wherein the TGF is TGF-β.

35. The graft collar of claim 24, wherein the first region contains chondrocytes.

36. The graft collar of claim 35, wherein the chondrocytes are BMSC-derived.

37. The graft collar of claim 24, wherein the first region contains stem cells.

38. The graft collar of claim 37, wherein the stem cells are BMSCs.

39. The graft collar of claim 24, wherein the biopolymer mesh is derived from at least one of collagen, chitosan, silk and alginate.

40. The graft collar of claim 24, wherein the biopolymer mesh is allogeneic or xenogenic.

41. The graft collar of claim 24, wherein the polymer-fiber mesh comprises aliphatic polyesters, poly(amino acids), copoly(ether-esters), polyalkylenes oxalates, polyamides, poly(iminocarbonates), polyorthoesters, polyoxaesters, polyamidoesters, poly(ε-caprolactone)ₛ, polyanhydrides, polyarylates, polyphosphazenes, polyhydroxyalkanoates, polysaccharides, biopolymers, poly(lactic-co-glycolic acid), poly(lactide), poly(glycolide) or a blend of two or more of the preceding polymers.

42. The graft collar of claim 24, wherein the polymer comprises at least one of the poly(lactide-co-glycolide), poly(lactide) and poly(glycolide).

43. The graft collar of claim 24, wherein the second region contains at least one of the following growth factors: BMP, IGF, VEGF and PDGF.

44. The graft collar of claim 43, wherein the BMP is BMP-2.

45. The graft collar of claim 24, wherein the second region includes osteoblasts and/or osteoblast-like cells.

46. The graft collar of claim 45, wherein the osteoblasts and/or osteoblast like cells are BMSC-derived.

47. The graft collar of claim 24, wherein the second region includes at least one of the following: osteogenic agents, osteogenic materials, osteoinductive agents, osteoinductive materials, osteoconductive agents, osteoconductive materials and chemical factors.

48. The graft collar of claim 24, wherein the second region contains nanoparticles of calcium phosphate.

49. The graft collar of claim 48, wherein the calcium phosphate is selected from the group comprising tricalcium phosphate, hydroxyapatite and a combination thereof.

50. The graft collar of claim 24, wherein the second region contains nanoparticles of bioglass.

51. The graft collar of claim 24, wherein the graft collar is biodegradable.

52. The graft collar of claim 24, wherein the graft collar is osteointegrative.

53. The graft collar of claim 53, wherein the graft collar is sutured around a tendon graft.

54. The graft collar of claim 50, wherein the tendon graft is a bone-patellar tendon-bone (BPTB) graft, a patellar tendon graft, a semitendinosus, a hamstring-tendon (HST) graft, a hamstring tendon graft, a quadriceps tendon graft, Achilles graft or tibialis graft.

55. The graft collar of claim 53, wherein the tendon graft is an allograft or an autograft.

56. A graft collar for fixing tendon to bone in a subject, wherein said graft collar comprises a sheet of mesh comprising fibers aligned substantially perpendicular in relation to a longitudinal axis of said tendon, wherein said mesh applies compression to the graft.

57. The graft collar of claim 56, wherein the mesh comprises a biopolymer.

58. The graft collar of claim 56, wherein the mesh comprises a polymer-fiber.

59. The graft collar of claim 56, wherein the graft collar comprises:
a) a first region comprising a mesh and hydrogel; and
b) a second region adjoining the first region and comprising a mesh.

60. A graft collar for fixing tendon to bone in a subject, wherein said graft collar comprises a sheet of mesh comprising fibers aligned substantially parallel in relation to a longitudinal axis of said tendon, wherein said mesh applies lateral tension to the graft.

61. The graft collar of claim 60, wherein the mesh comprises a biopolymer.

62. The graft collar of claim 60, wherein the mesh comprises a polymer-fiber.

63. The graft collar of claim 60, wherein the graft collar comprises:
a) a first region comprising a mesh and hydrogel; and
b) a second region adjoining the first region and comprising a mesh

64. A scaffold apparatus for fixing musculoskeletal soft tissue to bone in a subject, said scaffold apparatus comprising two portions, wherein each of the two portions comprising first through third phases, wherein (i) the first phase comprises a material which promotes growth and proliferation of fibroblasts, (ii) the second phase adjacent to the first phase comprises a material which promotes growth and proliferation of chondroblasts, and (iii) the third phase adjacent to the second phase comprises a material which promotes the growth and proliferation of osteoblasts.

65. The scaffold apparatus of claim 64, wherein the two portions encase respective portions of a soft tissue graft.

66. The scaffold apparatus of claim 64, wherein the two portions, in combination, encase the entirety of a soft tissue graft on all sides.

67. The scaffold apparatus of claims 65, wherein the soft tissue graft is a bone-patellar tendon-bone (BPTB) graft, a patellar tendon graft, a semitendinosus, a hamstring-tendon (HST) graft, a hamstring tendon graft, a quadriceps tendon graft, Achilles graft or tibialis graft.

68. The graft collar of claims 65, wherein the soft tissue graft is an allograft or an autograft.

69. The scaffold apparatus of claim 64, wherein a degradable cell barrier is inserted between two adjacent ones of said first through third phases.

70. The scaffold apparatus of claim 69, wherein the degradable cell barrier comprises a nanofiber mesh.

71. The scaffold apparatus of claim 70, wherein the nanofiber mesh comprises polylactide-co-glycolide (PLGA).

72. The scaffold apparatus of claim 70, wherein the nanofiber mesh is electrospun.

73. An interference apparatus for affixing soft tissue to bone, comprising the scaffold apparatus of claim 64.

74. The interference apparatus of claim 73, wherein the interference apparatus is biomimetic.

75. The interference apparatus of claim 73, wherein the interference apparatus is biodegradable.

76. The interference apparatus of claim 73, wherein the interference apparatus is osteointegrative.

77. A scaffold apparatus for fixing musculoskeletal soft tissue to bone in a subject, said scaffold apparatus comprising (i) a first phase comprising a material which promotes growth and proliferation of fibroblasts, (ii) a second phase adjacent to the first phase comprising a material which promotes growth and proliferation of chondroblasts, and (iii) a third phase adjacent to the second phase comprising a material which promotes the growth and proliferation of osteoblasts, wherein a degradable cell barrier is inserted between two adjacent ones of said first through third phases.

78. The scaffold apparatus of claim 77, wherein the degradable cell barrier is a nanofiber mesh.

79. The scaffold apparatus of claim 78, wherein the nanofiber mesh comprises polylactide-co-glycolide (PLGA).

80. The scaffold apparatus of claim 78, wherein the nanofiber mesh is electrospun.

81. A scaffold apparatus for fixing musculoskeletal soft tissue to bone in a subject, said scaffold apparatus comprising (i) a first phase comprising a material which promotes growth and proliferation of fibroblasts, (ii) a second phase adjacent to the first phase comprising a material which promotes growth and proliferation of chondroblasts, and (iii) a third phase adjacent to the second phase comprising a material which promotes the growth and proliferation of osteoblasts, wherein said first phase coupled to a soft tissue graft.

82. The scaffold apparatus of claim 81, wherein the soft tissue graft is a graft for a ligament of the subject.

83. The scaffold apparatus of claim 82, wherein the ligament is an anterior cruciate ligament of the subject.

84. The scaffold apparatus of claim 81, wherein the soft tissue graft is a bone-patellar tendon-bone (BPTB) graft, a patellar tendon graft, a semitendinosus, a hamstring-tendon (HST) graft, a hamstring tendon graft, a quadriceps tendon graft, Achilles graft or tibialis graft.

85. The graft collar of claim 81, wherein the soft tissue graft is an allograft or an autograft.

86. The scaffold apparatus of claim 81, wherein a portion of the scaffold apparatus is configured to be at least partially inserted into a femur of the subject and another portion of the scaffold apparatus is configured to be at least partially inserted into a tibia of the subject.

87. The scaffold apparatus of claim 81, wherein the scaffold apparatus is configured to be inserted in a femur of the subject through a tunnel.

88. The scaffold apparatus of claim 81, wherein the scaffold apparatus is configured to be inserted in a tibia of the subject through a tunnel.

89. The scaffold apparatus of claim 81, wherein the first phase is exposed to a joint cavity of the subject.

90. The scaffold apparatus of claim 81, wherein the second phase is positioned in proximate contact to articular cartilage of the subject.

91. The scaffold apparatus of claim 81, wherein the third phase is encased in bone tissue of the subject.

92. A scaffold apparatus for fixing musculoskeletal soft tissue to bone in a subject, said scaffold apparatus comprising (i) a graft collar and (ii) a polymer-fiber mesh coupled to the graft collar to apply mechanical loading to the graft collar.

93. The scaffold apparatus of claim 92, wherein the mechanical loading is compression.

94. The scaffold apparatus of claim 92, wherein the mechanical loading is tension.

95. The scaffold apparatus of claim 92, wherein the polymer-fiber mesh wraps around the graft collar.

96. The scaffold apparatus of claim 92, wherein an outer surface of the graft collar is wrapped in its entirety by the polymer-fiber mesh.

97. The scaffold apparatus of claim 92, wherein the graft collar is biphasic.

98. The scaffold apparatus of claim 92, wherein the biphasic graft collar includes a first phase comprising a material which promotes growth and proliferation of chondrocytes, and a second phase adjacent to the first phase comprising a material which promotes the growth and proliferation of osteoblasts.

99. The scaffold apparatus of claim 92, wherein the polymer-fiber mesh comprises nanofibers.

100. The scaffold apparatus of claim 99, wherein the nanofiber mesh comprises polylactide-co-glycolide (PLGA).

101. The scaffold apparatus of claim 99, wherein the nanofiber mesh is electrospun.

102. The scaffold apparatus of claim 92, wherein the scaffold apparatus is coupled to a soft tissue graft.

103. The apparatus of claim 102, wherein the soft tissue graft is a graft for a ligament of the subject.

104. The apparatus of claim 103, wherein the ligament is an anterior cruciate ligament of the subject.

105. The apparatus of claim 102, wherein the soft tissue graft is a bone-patellar tendon-bone (BPTB) graft, a patellar tendon graft, a semitendinosus, a hamstring-tendon (HST) graft, a hamstring tendon graft, a quadriceps tendon graft, Achilles graft or tibialis graft.

106. The graft collar of claim 102, wherein the soft tissue graft is an allograft or an autograft.

107. A graft-fixation apparatus comprising the scaffold apparatus of claim 102.

108. The apparatus of claim 107, wherein the graft fixation apparatus is an interference screw.

109. A scaffold apparatus for fixing musculoskeletal soft tissue to bone, said scaffold apparatus being configured to apply mechanical loading to a soft tissue graft to promote regeneration of a fibrocartilage interface between said soft tissue and said bone.

110. The scaffold apparatus of claim 109, wherein the soft tissue graft is a bone-patellar tendon-bone (BPTB) graft, a patellar tendon graft, a semitendinosus, a hamstring-tendon (HST) graft, a hamstring tendon graft, a quadriceps tendon graft, Achilles graft or tibialis graft.

111. The graft collar of claim 109, wherein the soft tissue graft is an allograft or an autograft.

112. The scaffold apparatus of claim 109, wherein the mechanical loading is compression.

113. scaffold apparatus of claim 109, wherein the mechanical loading is tension.

114. The scaffold apparatus of claim 109, wherein said scaffold apparatus comprises a nanofiber mesh configured to apply said mechanical loading to said soft tissue graft.

115. The scaffold apparatus of claim 109, wherein said mechanical loading is applied by said scaffold apparatus dynamically or intermittently to said soft tissue graft.

116. The scaffold apparatus of claim 109, wherein said mechanical loading is applied by said scaffold apparatus statically to promote regeneration of a fibrocartilage interface between said soft tissue and said bone in a subject.

117. The scaffold apparatus of claim 109, wherein said scaffold apparatus comprises a material that promotes growth and proliferation of chondroblasts.

118. The scaffold apparatus of claim 109, wherein said scaffold apparatus comprises first and second phases, wherein (i) the first phase comprises a material that promotes growth and proliferation of chondroblasts, (ii) the second phase adjacent to the first phase comprises a material that promotes growth and proliferation of osteoblasts.

119. The scaffold apparatus of claim 109, wherein said scaffold apparatus comprises first, second and third phases, wherein (i) the first phase comprises a material that promotes growth and proliferation of fibroblasts, (ii) the second phase adjacent to the first phase comprises a material that promotes growth and proliferation of chondroblasts, and (iii) the third phase adjacent to the second phase comprises a material that promotes the growth and proliferation of osteoblasts.

120. An apparatus for inducing formation of fibrocartilage, said apparatus comprising a graft collar having a hollow central portion along a longitudinal axis, wherein an outer surface of the graft collar is wrapped with a polymer-fiber mesh configured to apply mechanical loading to the graft collar.

121. The apparatus of claim 120, wherein the mechanical loading is compression.

122. The apparatus of claim 120, wherein the mechanical loading is tension.

123. The apparatus of claim 120, wherein the graft collar has a cylindrical body.

124. The apparatus of claim 120, wherein the graft collar includes a sliced cut parallel to a longitudinal axis

125. The apparatus of claim 120, wherein the outer surface of the graft collar is wrapped in its entirety.

126. The apparatus of claim 120, wherein the polymer-fiber mesh comprises nanofibers.

127. The apparatus of claim 126, wherein the nanofibers are aligned.

128. The apparatus of claim 127, wherein the nanofibers are aligned perpendicular to the longitudinal axis of the graft collar.

129. The apparatus of claim 126, wherein the nanofibers are unaligned.

130. The apparatus of claim 120, wherein the graft collar includes at least one of the following substances: anti-infectives, antibiotics, bisphosphonate, hormones, analgesics, anti-inflammatory agents, growth factors, angiogenic factors, chemotherapeutic agents, anti-rejection agents, and RGD peptides.

131. The apparatus of claim 120, wherein the growth factors are selected from the group consisting of TGFs, BMPs, IGFs, VEGFs and PDGFs.

132. The apparatus of claim 131, wherein the TGF is TGF-β.

133. The apparatus of claim 131, wherein the BMP is BMP-2.

134. The apparatus of claim 120, wherein the graft collar includes one or more of the following types of cells: chondrocytes, osteoblasts, osteoblast-like cells and stem cells.

135. The apparatus of claim 120, wherein the graft collar includes at least one of the following: osteogenic agents, osteogenic materials, osteoinductive agents, osteoinductive materials, osteoconductive agents, osteoconductive materials and chemical factors.

136. The apparatus of claim 120, wherein the polymer-fiber mesh comprises aliphatic polyesters, poly(amino acids), copoly(ether-esters), polyalkylenes oxalates, polyamides, poly(iminocarbonates), polyorthoesters, polyoxaesters, polyamidoesters, poly(ε-caprolactone)s, polyanhydrides, polyarylates, polyphosphazenes, polyhydroxyalkanoates, polysaccharides, biopolymers, poly(lactic-co-glycolic acid), poly(lactide), poly(glycolide) or a blend of two or more of the preceding polymers.

137. The apparatus of claim 120, wherein the polymer comprises at least one of the poly(lactic-co-glycolic acid), poly(lactide) and poly(glycolide).

138. The apparatus of claim 120, wherein the polymer-fiber mesh is 35% poly(DL-lactide-co-glycolic acid) 85:15, 55% N,N-dimethylformamide, and 10% ethanol.

139. The apparatus of claim 120, wherein the polymer-fiber mesh comprises particulate reinforcers.

140. The apparatus of claim 139, wherein the particulate reinforcers comprise nanoparticles.

141. The apparatus of claim 120, wherein the graft collar is porous.

142. The apparatus of claim 120, wherein the graft collar is lyophilized.

143. The apparatus of claim 120, wherein the graft collar is biodegradable.

144. The apparatus of claim 120, wherein the graft collar is osteointegrative.

145. The apparatus of claim 120, wherein the graft collar is composed of microspheres.

146. The apparatus of claim 145, wherein the microspheres comprise poly(DL-lactide-co-glycolic acid).

147. The apparatus of claim 145, wherein the microspheres comprise poly(DL-lactide-co-glycolic acid) and bioactive glass.

148. The apparatus of claim 120, wherein the apparatus further comprises a device which applies static loading to the graft collar.

149. The apparatus of claim 148, wherein the device is a clamp.

150. The apparatus of claim 120, wherein the mechanical loading provided by said graft collar is adjusted based on polymer composition.

151. The apparatus of claim 120, wherein the mechanical loading provided by said graft collar is adjusted based on fiber composition.

152. The apparatus of claim 120, wherein the mechanical loading provided by said graft collar is adjusted based on fiber alignment

153. The apparatus of claim 120, wherein the graft collar comprises:
(a) a first region comprising a polymer-fiber mesh and hydrogel; and
(b) a second region adjoining the first region and comprising polymer microspheres.

154. The apparatus of claim 153, wherein the first region supports the growth and maintenance of an interfacial zone between tendon and bone, and the second region supports the growth and maintenance of bone tissue.

155. The apparatus of claim 153, wherein the graft collar includes at least one of the following substances: anti-infectives, antibiotics, bisphosphonate, hormones, analgesics, anti-inflammatory agents, growth factors, angiogenic factors, chemotherapeutic agents, anti-rejections agents, and RGD peptides.

156. The apparatus of claim 153, wherein the hydrogel is photopolymerized, thermoset or chemically cross-linked.

157. The apparatus of claim 156, wherein the hydrogel is polyethylene glycol.

158. The apparatus of claim 153, wherein the polymer-fiber mesh comprises aligned fibers.

159. The apparatus of claim 153, wherein the polymer-fiber comprises unaligned fibers.

160. The apparatus of claim 153, wherein the first region contains TGF.

161. The apparatus of claim 160, wherein the TGF is TGF-β.

162. The apparatus of claim 153, wherein the first region contains chondrocytes.

163. The apparatus of claim 162, wherein the chondrocytes are BMSC-derived.

164. The apparatus of claim 153, wherein the first region contains stem cells.

165. The apparatus of claim 164, wherein the stem cells are BMSCs.

166. The apparatus of claim 153, wherein the second region contains at least one of the following growth factors: BMP, IGF, VEGF and PDGF.

167. The apparatus of claim 166, wherein the BMP is BMP-2.

168. The apparatus of claim 153, wherein the second region includes osteoblasts and/or osteoblast-like cells.

169. The apparatus of claim 168, wherein the osteoblasts and/or osteoblast like cells are BMSC-derived.

170. The apparatus of claim 153, wherein the second region includes at least one of the following: osteogenic agents, osteogenic materials, osteoinductive agents, osteoinductive materials, osteoconductive agents, osteoconductive materials and chemical factors.

171. The apparatus of claim 153, wherein the microspheres comprise poly(DL-lactide-co-glycolic acid).

172. The apparatus of claim 153, wherein the microspheres comprise poly(DL-lactide-co-glycolic acid) and bioactive glass.

173. The apparatus of claim 153, wherein the second region contains nanoparticles of calcium phosphate.

174. The apparatus of claim 173, wherein the calcium phosphate is selected from the group comprising tricalcium phosphate, hydroxyapatite and a combination thereof.

175. The apparatus of claim 153, wherein the second region contains nanoparticles of bioactive glass.

176. The apparatus of claim 153, wherein the graft collar is biodegradable.

177. The apparatus of claim 153, wherein the graft collar is osteointegrative.

178. A method for making a device for inducing formation of fibrocartilage comprising:
(a) forming a graft collar; and
(b) wrapping the graft collar prepared in step (a) with a polymer-fiber mesh, to form said device.

179. The method of claim 178, wherein said step (a) comprises:
(a1) processing a plurality of microspheres;
(a2) laying the microspheres processed in step (a) in a mold; and
(a3) sintering together the microspheres in the mold above a glass transition temperature.

180. The method of claim 179, wherein the microspheres further comprise bioactive glass.

181. The method of claim 178, wherein the polymer-fiber mesh comprises nanofibers.

182. The method of claim 178, wherein the polymer-fiber mesh comprises aliphatic polyesters, poly(amino acids), copoly(ether-esters), polyalkylenes oxalates, polyamides, poly(iminocarbonates), polyorthoesters, polyoxaesters, polyamidoesters, poly(ε-caprolactone)s, polyanhydrides, polyarylates, polyphosphazenes, polyhydroxyalkanoates, polysaccharides, biopolymers, poly(lactic-co-glycolic acid), poly(lactide), poly(glycolide) or a blend of two or more of the preceding polymers.

183. The method of claim 178, wherein the polymer-fiber mesh comprises at least one of the poly(lactic-co-glycolic acid), poly(lactide) and poly(glycolide).

184. The method of claim 178, wherein the polymer-fiber mesh is 35% poly(DL-lactide-co-glycolic acid) 85:15, 55% N,N-dimethylformamide, and 10% ethanol.

185. The method of claim 178, wherein the polymer-fiber mesh comprises particulate reinforcers.

186. The method of claim 189, wherein the particulate reinforcers comprise nanoparticles.

187. The method of claim 181, wherein the nanofibers wrapped around the graft collar are perpendicular to the longitudinal axis of the graft collar.

188. The method of claim 178, further comprising incubating the polymer-fiber mesh-wrapped graft collar at a suitable temperature, time and humidity to allow sintering of the polymer-fiber mesh to the graft collar.

189. The method of claim 188, wherein the polymer-fiber-mesh-wrapped graft collar is incubated at or around 37° C. and at or around 5% CO₂.

190. A method for inducing formation of fibrocartilage comprising enclosing a graft within a polymer-fiber mesh-wrapped graft collar configured to apply mechanical loading to the graft.

191. The method of claim 190, wherein the graft is a bone-patellar tendon-bone (BPTB) graft, a patellar tendon graft, a semitendinosus, a hamstring-tendon (HST) graft, a hamstring tendon graft, a quadriceps tendon graft, Achilles graft or tibialis graft.

192. The graft collar of claim 190, wherein the graft is an allograft or an autograft.

193. The method of claim 190, wherein the mechanical loading is compression.

194. The method of claim 190, further comprising:
a) replacing the mesh wrapping the graft collar with a new polymer-fiber mesh periodically to apply static compression to the graft.

195. The method of claim 194, comprising:
a) replacing the mesh wrapping the graft collar with a new polymer-fiber mesh every 24 hours to apply static compression to the graft.

196. The method of claim 190, further comprising:
a) removing the polymer-fiber mesh after a first predetermined period of time,
b) allowing the graft to rest for a second predetermined period of time,
c) wrapping the graft collar with a new polymer-fiber mesh, and
d) repeating steps a)-c),
so as to apply dynamic compression to the graft.

197. The method of claim 196, comprising:
a) removing the polymer-fiber mesh after a 24 hours,
b) allowing the graft to rest for 24 hours,
c) wrapping the graft collar with a new polymer-fiber mesh, and
d) repeating steps a)-c),
so as to apply dynamic compression to the graft.

198. The method of claim 190, wherein the mechanical loading is tension.

199. An apparatus for inducing formation of fibrocartilage, said apparatus comprising a graft collar having a hollow central portion along a longitudinal axis wherein an outer surface of the graft collar is clamped by a clamp to apply mechanical loading to the graft collar.

200. A method for inducing formation of fibrocartilage, said method comprising wrapping a polymer-fiber mesh circumferentially around a graft to apply mechanical loading to the graft.

201. The method of claim 178, wherein the graft is a bone-patellar tendon-bone (BPTB) graft, a patellar tendon graft, a semitendinosus, a hamstring-tendon (HST) graft, a hamstring tendon graft, a quadriceps tendon graft, Achilles graft or tibialis graft.

202. The graft collar of claim 178, wherein the graft is an allograft or an autograft.

203. An apparatus for inducing formation of fibrocartilage said apparatus comprising a graft and a polymer-fiber mesh wrapped circumferentially around the graft to apply mechanical loading to the graft.

204. The apparatus of claim 203, wherein the graft is a bone-patellar tendon-bone (BPTB) graft, a patellar tendon graft, a semitendinosus, a hamstring-tendon (HST) graft, a hamstring tendon graft, a quadriceps tendon graft, Achilles graft or tibialis graft.

205. The graft collar of claim 203, wherein the graft is an allograft or an autograft.

206. The method of claim 203, wherein the mechanical loading is compression.

207. The method of claim 203, further comprising:
a) replacing the mesh wrapping the graft with a new polymer-fiber mesh periodically to apply static compression to the graft.

208. The method of claim 207, comprising:
a) replacing the mesh wrapping the graft with a new polymer-fiber mesh every 24 hours to apply static compression to the graft.

209. The method of claim 203, further comprising:
a) removing the polymer-fiber mesh after a first predetermined period of time,
b) allowing the graft to rest for a second predetermined period of time,
c) wrapping the graft with a new polymer-fiber mesh, and
d) repeating steps a)-c),
so as to apply dynamic compression to the graft.

210. The method of claim 209, comprising:
a) removing the polymer-fiber mesh after a 24 hours,
b) allowing the graft to rest for 24 hours,
c) wrapping the graft with a new polymer-fiber mesh, and
d) repeating steps a)-c),
so as to apply dynamic compression to the graft.

211. The method of claim 203, wherein the mechanical loading is tension.
